SEQUENCE LISTING (8 sequences)

SEQ ID NO: 1 - DNA, 2279 bp, Homo sapiens
gctgacggggtggcagtgcggcgggttacggcctggtcagaccataatgacttcagcaaataaagcaatcgaattacaactacaagtgaaacaaaatgcagaagaattacaagactttatgcgggatttagaaaactgggaaaaagacattaaacaaaaggatatggaactaagaagacagaatggtgttcctgaagagaatttacctcctattcgaaatgggaattttaggaaaaagaagaaaggcaaagctaaagagtcttccaaaaaaaccagagaggaaaacacaaaaaacaggataaaatcttatgattatgaggcatgggcaaaacttgatgtggaccgtatccttgatgagcttgacaaagacgatagtacccatgagtctctgtctcaagaatcagagtcggaagaagatgggattcatgtagattcacaaaaggctcttgttttaaaagaaaagggcaataaatacttcaaacaaggaaaatatgatgaagcaattgactgctacacaaaaggcatggatgccgatccatataatcccgtgttgccaacgaacagagcgtcagcatattttagactgaaaaaatttgctgttgctgagtctgattgtaatttagcagttgccttgaatagaagttatacaaaggcttattccagacgaggtgctgctcgatttgctttgcaaaaattagaagaggccaaaaaagattatgaaagagtattagaactagaaccaaataactttgaagcaacaaatgaactcaggaaaatcagtcaggctttagcatccaaagaaaactcatatccaaaggaagctgacatagtgattaagtcaacagaaggagagcgaaagcaaattgaagcacaacagaataagcagcaggccatttcagagaaagatcgggggaatggatttttcaaagaggggaaatatgaaagagcaattgaatgctatactcgagggatagcagcagatggtgctaatgcccttcttccagctaacagagctatggcctatctgaagattcagaaatatgaagaagctgaaaaagactgcacacaagccattttattagatggctcatattctaaagcttttgccagaagaggaactgcaagaacatttttgggaaagctaaatgaggcaaaacaagattttgaaactgttttacttctggaacctggaaataagcaagcagtaactgaactctccaaaattaaaaaggaattaattgagaaaggacactgggatgatgtctttcttgattccacacaaagacaaaatgtggtaaaacccattgataatccaccgcatcctggatcaactaaaccactcaagaaggttattattgaagaaactggtaatttgatacagactattgatgtgccagatagcactactgctgctgctccagagaataatcctattaatctagcaaatgtaatagcagccacaggcaccacaagtaagaagaattcaagccaagatgtcctttttcccacaagtgatactccaagagcaaaagtattgaaaatagaagaagtcagtgatacttcatccctgcaacctcaagccagtttgaagcaggatgtatgtcagtcttacagcgagaaaatgcccatagagatagaacaaaaacctgctcagtttgccacaactgttcttcctccaattcctgcaaactcgttccagctcgaatctgatttcagacaattgaaaagttctccagatatgttgtatcagtatttaaagcaaattgaaccatctttgtatcctaagttgtttcagaaaaatctggatccagatgtattcaaccagatcgttaaaattctgcatgacttttacattgagaaagaaaagccattactcatctttgaaatcttacaaagactttctgaactaaaaaggtttgatatggcagtgatgtttatgtcagaaacagagaaaaagattgcacgtgcattatttaatcacatagacaagtcaggattgaaggatagttctgtcgaagaactcaagaaaagatacggtggttgatttccatttttgctgaaataattgtttttgactttcatatgtaaattttttctactgaaagtgttttgctttttaagaaaatgaaattatatagcaggaaaggactatctttgaacataagttaattaactataaggtgaattgtgatttaactagtgagaattgtattcaagtgaactctgtttttctgaaaataaaaatataaacaatgagaaaaaaaaaaaaaaaaaaaaaa

SEQ ID NO: 2 - protein, 665 aa, Homo sapiens
MTSANKAIELQLQVKQNAEELQDFMRDLENWEKDIKQKDMELRRQNGVPEENLPPIRNGNFRKKKKGKAKESSKKTREENTKNRIKSYDYEAWAKLDVDRILDELDKDDSTHESLSQESESEEDGIHVDSQKALVLKEKGNKYFKQGKYDEAIDCYTKGMDADPYNPVLPTNRASAYFRLKKFAVAESDCNLAVALNRSYTKAYSRRGAARFALQKLEEAKKDYERVLELEPNNFEATNELRKISQALASKENSYPKEADIVIKSTEGERKQIEAQQNKQQAISEKDRGNGFFKEGKYERAIECYTRGIAADGANALLPANRAMAYLKIQKYEEAEKDCTQAILLDGSYSKAFARRGTARTFLGKLNEAKQDFETVLLLEPGNKQAVTELSKIKKELIEKGHWDDVFLDSTQRQNVVKPIDNPPHPGSTKPLKKVIIEETGNLIQTIDVPDSTTAAAPENNPINLANVIAATGTTSKKNSSQDVLFPTSDTPRAKVLKIEEVSDTSSLQPQASLKQDVCQSYSEKMPIEIEQKPAQFATTVLPPIPANSFQLESDFRQLKSSPDMLYQYLKQIEPSLYPKLFQKNLDPDVFNQIVKILHDFYIEKEKPLLIFEILQRLSELKRFDMAVMFMSETEKKIARALFNHIDKSGLKDSSVEELKKRYGG

SEQ ID NO: 3 - protein, 332 aa, Homo sapiens
QKYEEAEKDCTQAILLDGSYSKAFARRGTARTFLGKLNEAKQDFETVLLLEPGNKQAVTELSKIKKELIEKGHWDDVFLDSTQRQNVVKPIDNPPHPGSTKPLKKVIIEETGNLIQTIDVPDSTTAAAPENNPINLANVIAATGTTSKKNSSQDVLFPTSDTPRAKVLKIEEVSDTSSLQPQASLKQDVCQSYSEKMPIEIEQKPAQFATTVLPPIPANSFQLESDFRQLKSSPDMLYQYLKQIEPSLYPKLFQKNLDPDVFNQIVKILHDFYIEKEKPLLIFEILQRLSELKRFDMAVMFMSETEKKIARALFNHIDKSGLKDSSVEELKK

SEQ ID NO: 4 - DNA, 49 bp, Artificial sequence
  Linker-primer designed with a GAGA sequence to protect the Xho I restriction site and an 18-base (dT) sequence
gagagagagagagagagaaactagtctcgagtttttttttttttttttt

SEQ ID NO: 5 - DNA, 13 bp, Artificial sequence
  Synthetic oligonucleotide sequence for 5′ to 3′ primer used in construction of Lambda ZAP EcoR I/Xho I 43HIV cDNA Library
aattcggcacgag

SEQ ID NO: 6 - DNA, 8 bp, Artificial sequence
  Synthetic oligonucleotide sequence for 3′ to 5′ primer used in construction of Lambda ZAP EcoR I/Xho I 43HIV cDNA Library
gccgtgtc

SEQ ID NO: 7 - DNA, 24 bp, Artificial sequence
  Synthetic PCR primer for amplification of FL14676485 upstream primer
tagaaaactgggaaaaagacatta

SEQ ID NO: 8 - DNA, 17 bp, Artificial sequence
  Synthetic PCR primer for amplification of FL14676485 downstream primer
ttggcaacacgggatta

(19) United States
(12) Patent Application Publication
(10) Pub. No.: US 2004/0197770 A1
(43) Pub. Date: Oct. 7, 2004

(54) Induction of apoptosis by HIV-1 infected monocytic cells

(75) Inventors: Kirk Sperber, Bronxville, NY (US); Irwin H. Gelman, Buffalo, NY (US)

Correspondence Address: MARSHALL, GERSTEIN & BORUN LLP, 6300 SEARS TOWER, 233 S. WACKER DRIVE, CHICAGO, IL, 60606, US

(73) Assignee: MOUNT SINAI SCHOOL OF MEDICINE, New York, NY

(21) Appl. No.: 10/712,671

(22) Filed: Nov. 13, 2003

Related U.S. Application Data
(60) Provisional application No. 60/426,103, filed on Nov. 14, 2002.

(51) Int. Cl. C12Q 1/70

(52) U.S. Cl. 435/5

(57) ABSTRACT

The present invention generally relates to the treatment or inhibition of diseases associated with HIV-1 infection. In particular, the present invention identifies a protein, which is secreted by macrophages as a result of HIV infection. The secreted protein induces apoptosis in neuronal cells, as well as T cells and B cell. The protein is specifically expressed in the neuronal tissue of HAD patients but not in the neuronal tissue of patients with non-HIV associated dementia. Methods and compositions for decreasing, inhibiting, or otherwise abrogating neuronal cell apoptosis that leads to HIV-1 associated dementia are described.

### STATEMENT OF RELATED CASES AND GOVERNMENT FUNDING

The present application claims the benefit of priority under 35 U.S.C. § 119 of U.S. Provisional Patent Application No. 60/426,103, filed Nov. 14, 2002. The entire text of the foregoing application is incorporated herein by reference. Some experimental data described herein was generated with the support of National Institutes of Health grants AI45343 and AI44236 both awarded to Kirk Sperber.

### BACKGROUND

1. Field of the Invention

The present invention generally relates to the treatment or inhibition of diseases associated with HIV-1 infection. In particular, the present invention provides methods and compositions for decreasing, inhibiting, or otherwise abrogating neuronal cell apoptosis that leads to HIV-1 associated dementia. In addition, the compositions of the present invention may be used systemically for the treatment of HIV to abrogate T and B-cell apoptosis. The compositions of the present invention also may be used to ameliorate inflammatory disorders by inducing cell death in such disorders.

2. Background of the Invention

In 1981, acquired immune deficiency syndrome (AIDS) was identified as a disease that severely compromises the human immune system and, that almost without exception, leads to death. In 1983, the etiological cause of AIDS was determined to be the human immunodeficiency virus (HIV). Global statistics (UNAIDS: Report on the Global HIV/AIDS Epidemic, December 1998), indicated that in 1998 as many as 33 million people worldwide were infected with the virus. And the virus continues to spread.

Despite the devastation caused by this virus, there are promising avenues for therapeutic intervention that have provided means of controlling the infection. There are three classes of therapeutic agents available. Firstly, competitive inhibitors of apartyl protease such as, e.g., saquinavir, indinavir, ritonavir, nelfinavir and amprenavir have been used. Secondly, reverse transcriptase inhibitors such as, zidovudine, didanosine, stavudine, lamivudine, zalcitabine and abacavir, which act as substrates for the reverse transcriptase and interrupt viral cDNA synthesis also have proven effective. Thirdly, non-nucleoside reverse transcriptase inhibitors, nevaripine, delavaridine and efavirenz inhibit the synthesis of viral cDNA via a non-competitive (or uncompetitive) mechanism. All three classes of drugs have been separately employed to reduce viral replication. However, such therapeutic intervention has been marred by the fact that the virus rapidly evolves to develop resistance to these agents.

An approach that has proved useful in combating both virus infection and suppression of emergence of resistance is to employ a triple-drug combination antiretroviral therapy. In the US, where combination therapy is widely available, the number of HIV-related deaths has declined as a result of this intervention (Palella et al., Engl. J. Med. 338, 853, 1998; Hammer et al., N Engl J Med., 337:725-733, 1997; Cameron et al., Lancet, 351:543-549, 1998; Montaner et al., JAMA, 279:930-937, 1998). As a result, triple-drug regimens have been widely adopted for the treatment of HIV infection starting in 1996 (Carpenter et al., JAMA, 283:381-390, 2000; Gazzard et al., Lancet, 1998;352:314-316, 1998; Guidelines for the Use of Antiretroviral Agents in HIV-Infected Adults and Adolescents. Washington, D.C.: US Dept of Health and Human Services/Henry J. Kaiser Family Foundation; January 2000). Through the use of powerful triple-drug cocktails, the prognosis for HIV-infected patients has improved markedly.

Unfortunately, along with the increased life-expectancy of HIV-infected patients, these patients increasingly develop diseases associated with prolonged HIV-1 infection. These diseases seem to result from the expression of proteins produced upon infection of HIV-1. Often, the expression of such proteins and related deleterious effects manifest even in the absence of detectable ongoing viral replication. Thus, as HIV infection is being turned from predictable AIDS into a maintenance disease, the new challenge for clinicians becomes a question of controlling the emergence of these HIV-induced diseases. One such disease is HIV-1 associated dementia (HAD).

HAD is a metabolic encephalopathy induced by viral infection and fueled by immune activation of brain mononuclear phagocytes (perivascular and parenchymal macrophages and microglia) (For review, see Diesing et al., AIDS Read., 12(8):358-68, 2002). These same cells serve as reservoirs for persistent infection and sources for soluble neurotoxins. HAD is characterized by impaired cognitive, behavioral, and motor functions. The cognitive abnormalities associated with HAD may manifest years after initial viral exposure and are associated with depletion of CD4+ T lymphocytes and high viral loads.

While the triple-combination therapies (sometimes referred to as highly active antiretroviral therapy (HAART)) discussed above have ameliorated HIV infection and drug resistance, the cognitive dysfunction associated with HAD remains a cause of morbidity in many infected individuals. Thus, while HAART has resulted in a decrease in the incidence of HAD (Dore et al., J. Acquir. Immune. Defic. Syndr. Hum. Retrovirol. 16, 39-43, Ferrando et al., AIDS 12:F65-F70, 1998), it does not seem to provide complete protection from or reversal of HAD (Dore et al., AIDS 13:1249-1253, 1999, Major et al., Science 288: 440-442, 2000). In addition, the prevalence of the dementia may eventually increase as people live longer with AIDS (Lipton et al., N. Engl. J. Med. 332:934-940, 1995, Gartner, Science 287:602-604, 2000). Currently there is no specific treatment for HAD, mainly because of an incomplete understanding of how HIV infection causes neuronal injury and apoptosis.

The principal pathway for HIV entry into the central nervous system (CNS) is through infected monocytes. The predominant pathogenesis of HAD is believed to involve activation of monocytic cells (macrophages and microglia) and their subsequent release of toxins that lead to neuronal and astrocytic dysfunction. Macrophages and microglia can be activated by HIV infection itself, by interaction with viral proteins, or by immune stimulation due to concurrent infection or other factors (Lipton et al., N. Engl. J. Med. 332:934-940, 1995). It is possible that direct effects of viral proteins on neurons may also contribute to neurodegeneration, although neurons themselves are not infected by HIV-1.

HIV enters the CNS early in the course of infection, and the virus resides primarily in microglia and macrophages. However, infection of these cells may not be sufficient to initiate neurodegeneration (Gartner, Science 287:602-604, 2000). It has been proposed that factors associated with advanced HIV infection in the periphery (non-CNS) are important triggers for events leading to dementia. One such factor could be the increased number of circulating monocytes that express CD16 and CD69. These activated cells adhere to the normal endothelium of the brain microvasculature, transmigrate, and then trigger a number of deleterious processes crucial in HIV infection of the CNS (Gartner, Science 287:602-604, 2000, Asensio et al., Trends. Neurosci. 22:504-512, 1999). Microglial and astrocytic chemokines (cell migration (chemotaxis)-inducing cytokines), such as monocyte chemoattractant protein (MCP)-1, seem to regulate migration of peripheral blood mononuclear cells through the blood-brain barrier (Gartner, Science 287:602-604, 2000).

Histological studies in specimens from HIV-1-infected humans and simian immunodeficiency virus (SIV)-infected rhesus Macaques show that lymphocytes and monocytes migrate into the brain (Prospero-Garcia et al., Proc. Natl. Acad. Sci. USA. 93:14158-14163, 1996, Kalams et al., Curr. Top. Microbiol. Immunol. 202:79-88, 1995). Cellular migration also involves adhesion molecules, and increased expression of vascular cell-adhesion molecule-1 (VCAM-1) has been implicated in mononuclear cell migration into the brain during HIV and SIV infection (Sasseville et al., Am. J. Pathol. 144:27-40, 1994). It has also been suggested that the inflammatory cytokine tumor-necrosis factor-α (TNF-α) opens a paracellular route for HIV-1 across the BBB (Sasseville et al., Am. J. Pathol. 144:27-40, 1994). These findings indicate that one reason why HAD rarely occurs before the onset of advanced HIV disease is that a vicious cycle of immune dysregulation and BBB dysfunction is required to achieve sufficient entry of infected or activated immune cells into the brain to cause neuronal injury.

The neuropathology associated with HIV infection in the brain, termed HIV encephalitis is characterized by widespread reactive astrocytosis, myelin pallor, and infiltration predominantly by monocytoid cells, including blood-derived macrophages, resident microglia and multinucleated giant cells. However, numbers of HIV-infected cells, multinucleated giant cells or viral antigen in CNS tissue do not correlate well with measures of cognitive function (Glass et al., Ann. Neurol. 38: 755-762, 1995, Masliah et al., Ann. Neurol. 42, 963-972, 1997). The pathological features most closely associated with the clinical signs of HAD include increased numbers of microglia (Glass et al., Ann. Neurol. 38: 755-762, 1995), elevated TNF-α messenger RNA in microglia and astrocytes (Wesselingh et al., J. Neuroimmunol. 74:1-8, 1997), evidence of excitotoxins (Masliah et al., Ann. Neurol. 42, 963-972, 1997, Wesselingh et al., J. Neuroimmunol. 74:1-8, 1997), decreased synaptic and dendritic density (Masliah et al., Ann. Neurol. 42, 963-972, 1997, Everall et al., Brain. Pathol. 9: 209-217, 1999), and selective neuronal loss (Fox et al., Neuropathol. Exp. Neurol. 56: 360-368, 1997, Masliah et al., Neuropathol. Exp. Neurol. 51:585-593, 1992). Several groups have demonstrated that HAD is associated with evidence of neuronal apoptosis (Adle-Biassette et al., Appl. Neurobiol. 21:218-227, 1995, Gelbard et al., Neuropathol. Appl. Neurobiol. 21: 208-217, 1995, Petito et al., Am. J. Pathol. 146:1121-1130, 1995), but this finding is not clearly associated with viral burden (Adle-Biassette et al., Appl. Neurobiol. 21:218-227, 1995) or a history of dementia (Adle-Biassette et al., Neuropathol. Appl. Neurobiol. 25:123-133, 1999).

The topographic distribution of neuronal apoptosis is correlated with evidence of structural atrophy and closely associated with markers of microglial activation, especially within subcortical deep gray structures (Adle-Biassette et al., Neuropathol. Appl. Neurobiol. 25:123-133, 1999), which may show a predilection for atrophy in HAD. The neuropathology observed in HAD, coupled with extensive research on both in vitro and animal models of HIV-induced neurodegeneration, have led to a complicated model for the pathogenesis of HAD. It is likely that a construct similar to the multi-hit model of oncogenesis will be the most effective way to understand all of the factors involved in the pathogenesis of HAD. Macrophages and microglia can be infected by HIV-1, but they can also be activated by factors released from infected cells, including cytokines and shed viral proteins such as gp120 (Aziz et al., Nature 338:505, 1989, Pope et al., Cell 78:389, 1994, Mosier et al., Science 260:689, 1993, Watanabe et al., J Virol 65:3853, 1991, Johnson et al., AIDS Res Human Retrovirures 9:375, 1993, Gendelman et al., J Virol 65:3865, 1991, Schuitemaker et al., J Infect Dis 168:1140, 1993).

Microglial activation affects all cell types in the CNS, resulting in upregulation of cytokines, chemokines and endothelial adhesion molecules (Lipton et al., N. Engl. J. Med. 332:934-940, 1995, Gartner, Science 287:602-604, 2000). Some of these molecules may contribute to neuronal damage and apoptosis through direct or indirect routes. In addition, activated microglia release excitatory amino acids (EAAs) and related substances, including glutamate, quinolinate, cysteine and the amine NTox. EAAs released by infected or activated microglia can induce neuronal apoptosis through a process known as excitotoxicity, which engenders excessive Ca2+ influx and free radical (nitric oxide and superoxide anion) formation by overstimulation of glutamate receptors. Some HIV proteins, such as gp120 and Tat, have also been reported to be directly neurotoxic, although high concentrations of viral protein may be needed or neurons may have to be cultured in isolation to see these direct effects (Meucci et al., Proc. Natl. Acad. Sci. USA 95:14500-14505, 1998, Liu et al., Nature Med. 6:1380-1387, 2000). Importantly, toxic viral proteins and factors released from microglia may act synergistically to promote neurodegeneration, even in the absence of extensive viral invasion of the CNS.

Macrophages and microglia are crucial in HAD because they are the only resident cells that can be productively infected with HIV-1 in the CNS (Lipton et al., N. Engl. J. Med. 332:934-940, 1995), although a non-productive or latent infection of astrocytes has been observed. HIV-1-infected macrophages migrate into the brain and constitute the principal route of viral entry into the CNS (Gartner, Science 287:602-604, 2000). HIV-infected or immune-stimulated macrophages/microglia produce neurotoxins, and macrophages/microglia are required for HIV-1- or gp120-induced neurotoxicity (Giulian et al., Science 250:1593-1596, 1990, Giulian et al., Proc. Natl. Acad. Sci. USA. 90:2769-2773, 1993, Dreyer et al., Science, 248: 364-367, 1990). Macrophage/microglia damage neurons by releasing excitotoxic substances that produce excessive activation of glutamate receptors, primarily of the N-methyl-D-aspartate subtype (NMDAR). In addition, indirect neurotoxicity is probably mediated by macrophage- and microglial-derived arachidonate and its metabolites including platelet-activating factor (PAF), free radicals chemokines and viral proteins (Meyaard et al., Science 257:217, 1992). Chemokine and cytokine signaling in microglia promote p38 MAPK activity that in turn phosphorylates/activates the transcription factor MEF2C. Pharmacological inhibition of p38 MAPK prevents microglial induction of TNF-α and inducible nitric oxide synthase (iNOS) gene expression in response to inflammatory stimuli (Bhat et al., J. Neurosci. 18: 1633-1641, 1998).

Although there is general agreement that HIV does not infect neurons, the primary cause of neuronal injury remains in question. Evidence supports multiple theories for neuronal injury by various viral proteins, including Tat, Nef, Vpr and the Env proteins gp120 and gp41. Two theories predominate and are best described as the ‘direct injury’ hypothesis and the ‘indirect’ or ‘bystander effect’ hypothesis. They are in no way mutually exclusive, and currently available data support a role for both theories, although an indirect form of neurotoxicity seems to have more support. Apoptotic neurons do not co-localize with infected microglia in HAD patients (Shi et al., J. Clin. Invest. 98:1979-1990, 1996), supporting the hypothesis the HIV infection causes neurodegeneration through the release of soluble factors. Systems designed to study the effect of soluble factors released from microglia have included mixed from human fetal brain directly infected with HIV (Shi et al., J. Clin. Invest. 98:1979-1990, 1996), severe combined immunodeficiency mice cerebrocortical cultures inoculated with HIV-infected human monocytes (Xiong et al., J. Neurovirol. 6: S14-S23, 2000), and mixed rodent cerebrocortical cultures exposed to very low concentrations of the envelope protein HIV/gp120.

Thus, while it is known that macrophages are intimately involved with the progression of HAD through apoptosis of neuronal cells (Sperber et al., J Immunol Methods 129:31, 1990; Sperber et al., AIDS Res Human Retroviruses 9:657, 1993; Yoo, et al., J Immunol 157:1313, 1996; Polyak et al., J Immunol 159:2177, 1997; Chen et al., J Immunology 161:4257, 1998; Rakoff-Nahoum et al., Journal of Immunology 167:2331, 2001; Chen et al., J Immunology 161:4257, 1998), little is known about the factor or factors responsible for this effect.

Furthermore, while HAART has been used to provide some control HIV infection, the underlying systemic HIV infection remains ongoing and has profound effects on T cell and B cell populations in the individual. For example, progressive depletion of CD4+ T cells is a characteristic feature of HIV-1 infection (Pantaleo et al., N Eng J Med 328:327, 1993). Both virologic and immunologic mechanisms have been implicated in the loss of CD4+ T cells. In addition, apoptosis has been proposed as an alternative explanation for T cell loss seen in HIV-1 infected individuals (Oyaizu and Pahwa. J Clin Immunol 15:217, 1995). For example, spontaneous apoptosis of CD4+ and CD8+T cells and activation-induced apoptosis have been reported in peripheral blood lymphocytes (PBMC) and lymph nodes during HIV-1 infection (Meyaard et al., Science 257:217, 1992; Groux et al., J Exp Med 175:331, 1992; Oyaizu et al., Blood 82:3392, 1993; Corbonari et al., Blood 83:1268, 1994; Sarin et al., J Immunol 153:862, 1994; Meyaard et al., J Clin Invest 93:982, 1994; Lewis et al., J Immunol 153:412, 1994).

The accelerated apoptosis may relate to cross linking of CD4 by gp120 leading to aberrant T cell signaling (Diamond et al., J Immunol 141:3715, 1988; Chirmule et al., Blood 75:152, 1990; Oyaizu et al., Proc Natl Acad Sci USA 84:2379, 1990), cytokines (Oyaizu and Pahwa. J Clin Immunol 15:217, 1995), Fas and FasL interactions (Debatin et al., Blood 83:3101, 1994; McClosky et al., Cytometry 22:111, 1995; Kabayoshi et al., Proc Natl Acad Sci USA 90:7573, 1990), superantigen activity encoded by HIV-1 products (Hugin et al., Science 252:424, 1991; Aziz et al., Nature 338:505, 1989) or the involvement of accessory cells. Several lines of evidence implicate accessory cells including monocytes and dendritic cells in the induction of apoptosis during the course of HIV-1 infection. Monocytes and dendritic cells serve as reservoirs for HIV-1 providing virions and the envelope protein gp120 to target CD4+ T cells (Pope et al., Cell 78:389, 1994). Antigen presenting cell dysfunction as a result of HIV-1 infection may cause defective T cell activation resulting in apoptosis instead of cellular activation (Mosier et al., Science 260:689, 1993; Watanabe et al., J Virol 65:3853, 1991; Johnson et al., AIDS Res Human Retrovirures 9:375, 1993; Gendelman et al., J Virol 65:3865, 1991; Schuitemaker et al., J Infect Dis 168:1140, 1993). HIV-1 infection or crosslinking of CD4 on monocytes results in the upregulation of FasL expression that could induce apoptosis in uninfected bystander CD4+ T cells (Badley et al., J Virol 70:199, 1996; Oyaizu et al., J Immunol 158:2456, 1997; Wu et al., Proc Natl Acad Sci USA 92:1525, 1994).

Soluble pro-apoptotic factors especially those produced by HIV-1 infected macrophages may also be playing a role in T cell depletion. Macrophages have been reported to produce pro-apoptotic chemokines and cytokines as well as apoptosis promoting low molecular weight molecules such as reactive oxygen species, prostaglandin and nitric oxide (Oyaizu and Pahwa. J Clin Immunol 15:217, 1995). The chemokine SDF-1 (stromal derived growth factor) which signals through the CXCR4 chemokine receptor delivers a death signal to CD8+T cells and to neuronal cell lines (Herbein et al., Nature 395: 189, 1998; Hesselgesser et al., Curr Biol 8: 595, 1998). SDF-1 blocks infection of T cells by T cell tropic viruses and may play an important role in the regulation of cell differentiation, proliferation, and migration of CD8+T cells in inflammatory responses (Ameisen Nature 395:117, 1998).

After HIV-1 infection, there is increased production of pro-inflammatory cytokines including IL-6, IFN-γ, TGF-β and TNF-α (Poli and Fauci, AIDS Res Hum Retroviruses 8:191, 1992). In HIV-1 infected individuals, this cytokine imbalance may contribute to apoptosis. TNF-α, TGF-β as well as IFN-γ promotes apoptosis (Zauli et al., J Exp Med 183:99, 1996; Clements and Stamenkouri. J Exp Med 180:557, 1994; Wang et al., J Immunol 152:3842, 1994; Grell et al., J Immunol 153:1963, 1993; Liu and Janeway. J Exp Med 172:1735, 1990; Groux et al., Eur J Immunol 23:1623, 1993; Novelli et al., J Immunol 152:496, 1994; Clerici et al., Proc Natl Acad Sci USA 91:11811, 1994). Thus, there is significant evidence that soluble proapoptotic factors are released upon HIV-1 infection and that these factors may cause the depletion of T and B cells in HIV-1 infected individuals.

There remains a need to identify the factors responsible for apoptosis in T and B cells as a result of HIV-1 infection. Once such factors are identified, it is possible to design therapeutic intervention strategies to combat systemic HIV-1 infection. In addition, to effectively combat secondary HIV-1 associated disorders, such as HAD, it will be a necessary to identify such factor responsible for triggering, causing or otherwise resulting in the symptoms of HAD.

### SUMMARY OF THE INVENTION

The present invention identifies a protein secreted by macrophages upon HIV infection. The secreted protein induces apoptosis in neuronal cells, as well as T cells and B cell. The protein is specifically expressed in the neuronal tissue of HAD patients but not in the neuronal tissue of patients with non-HIV associated dementia. The present invention describes methods and compositions for exploiting the protein and related compositions for decreasing, inhibiting, or otherwise abrogating neuronal cell apoptosis that leads to HIV-1 associated dementia as well as systemic treatment of HIV with compositions designed to inhibit or abrogate the T and B cell apoptosis induced by this protein. In addition the present invention is directed to methods and compositions of increasing, promoting or otherwise augmenting apoptosis in inflammatory disease. The protein used in the diagnostic or other methods of the present invention is FLJ21908 protein (now referred to herein throughout as SHIV A (soluble HIV apoptotic)) which comprises a sequence of SEQ ID NO:2. In preferred embodiments, the protein comprises a fragment of the full-length protein, such as for example a 6 kDa fragment of the protein of SEQ ID NO:2.

In specific embodiments, the present invention describes a method of diagnosing HIV infection in a subject comprising obtaining a biological sample from the subject; determining the increased expression of SHIV A in the biological sample. In specific embodiments, the cell sample contains cells selected from the group consisting of macrophages, neuronal cells, central nervous system cells, microglial cells, glial cells, T-cells, and B-cells.

In particular embodiments, the diagnostic determining step involves assaying for the presence of a nucleic acid that encodes the SHIV A protein in the sample. Such a determining step may further comprise subjecting the sample to conditions suitable for amplifying the nucleic acid. In other embodiments, the diagnostic determining step involves contacting the sample with an antibody that binds immunologically to a SHIV A protein. In exemplary embodiments, such a method may further comprise subjecting the sample to ELISA.

In the diagnostic methods of the present invention, it will be desirable to compare the expression of SHIV A in the subject with the expression of SHIV A in a non-HIV infected sample. Such comparison may comprise evaluating the level of expression of SHIV A, or evaluating the structure of the SHIV A gene, protein or transcript. In particular embodiments, the evaluating will comprise performing an assay selected from the group consisting of sequencing, nucleic acid hybridization, PCR, RNAase protection. More specifically, a nucleic acid hybridization assay in the evaluating step may be performed using a microarray comprising oligonucleotides derived from the sequence of SEQ ID NO:1. In such embodiments, the oligonucleotides are each at least 20 bases in length.

The present invention further contemplates a composition comprising an isolated polypeptide encoding a SHIV A protein having the sequence of SEQ ID NO:2 and an immunological adjuvant, or pharmaceutically acceptable carrier or diluent. In preferred embodiments, the composition may further comprise a combination of one or more competitive inhibitor of apartyl protease, one or more nucleoside substrate reverse transcriptase inhibitor, or one or more non-nucleoside reverse transcriptase inhibitors.

In preferred embodiments the polypeptide of the composition is conjugated to a carrier molecule or a tag. For example, the protein is tagged to a carrier molecule selected from the group consisting of KLH, and BSA.

Also contemplated by the present invention is a monoclonal antibody that binds immunologically to a SHIV A protein. More particularly, the monoclonal antibody binds to a protein of SEQ ID NO:2 or a fragment or variant thereof. In preferred embodiments, the monoclonal antibody binds to a 6 kDa fragment of the protein of SEQ ID NO:2. Preferably, the monoclonal antibody further binds to a protein of SEQ ID NO:3. In specific embodiments, the monoclonal antibody neutralizes the biological activity of the 6 kDa fragment of the protein of SEQ ID NO:2 or SEQ ID NO:3. In other embodiments, the monoclonal antibody does not bind immunologically to other human polypeptides. The monoclonal antibody may bind to non-human homologs of SHIV A and may be used to isolate and detect the same. The monoclonal antibody further may comprise a detectable label, such as for example a detectable label is selected from the group consisting of a fluorescent label, a chemiluminescent label, a radiolabel and an enzyme. In preferred embodiments, the monoclonal antibody is formulated into a pharmaceutical composition. In other preferred embodiments, the monoclonal antibody is formulated into a diagnostic kit, the kit further comprising instructions for performing a diagnostic assay to determine the presence of a SHIV A protein.

Also disclosed herein as part of the invention is a hybridoma cell that produces a monoclonal antibody that binds immunologically to a SHIV A protein. The hybridoma is one which produces a monoclonal antibody that does not bind to other human proteins, however, the antibody produced may bind to a non-human homolog of a protein of SEQ ID NO:2. The invention also contemplates polyclonal antisera comprising antibodies which bind immunologically to a SHIV A protein.

Also contemplated herein is a nucleic acid construct comprising a polynucleotide of SEQ ID NO:1 operably linked to a heterologous promoter. The promoter may be any promoter used in the recombinant expression of a protein that is heterologous to the endogenous promoter for the nucleic acid encoding a SHIV A protein. Exemplary heterologous promoters include but are not limited to CMV, RSV, SV40, UbC, EF1alpha, and tetracycline inducible promoter. In certain embodiments, the nucleic acid construct is one in which the polynucleotide of SEQ ID NO:1 or fragment thereof is positioned in an antisense orientation with respect to the heterologous promoter. In specific embodiments, the nucleic acid construct further comprises the nucleic acids of a viral vector selected from the group consisting of retrovirus, adenovirus, adeno-associated virus, herpes virus, and vaccinia virus. The nucleic acid construct may be packaged in a liposome.

In specific embodiments, the present invention contemplates an apoptotic protein comprising the sequence of SEQ ID NO:3, nucleic acid molecules and expression vectors that encode such a protein, as well as host cells transformed with such expression vectors.

Also described herein is a method of altering apoptosis in a first cell, comprising altering the expression or processing of SHIV A protein in a second cell. In preferred embodiments, the second cell is an HIV-infected cell, the first cell is a neuronal cell, and the altering comprises decreasing apoptosis in the first cell by inhibiting the expression or activity of SHIV A protein in the HIV-infected second cell. In other embodiments, the second cell is an HIV-infected cell, the first cell is a B cell or a T cell, and the altering comprises decreasing apoptosis in the first cell by inhibiting the expression or activity of SHIV A protein in the HIV-infected second cell. Such inhibition of expression of SHIV A is useful in the treatment of HIV and HIV associated disorders, in such embodiments, the first cell may be co-treated with HAART. In particular embodiments, the inhibition of expression of SHIV A may lead to a decrease in apoptosis in the HIV-1 infected second cell and/or a decrease in apoptosis in cells surrounding the HIV-1 infected second cell.

In certain embodiments, the first cell is a hyperproliferative cell and the altering comprises increasing cell apoptosis in the first cell by increasing the expression, processing or activity of SHIV A protein in the second cell. In such embodiments, the apoptosis may be increased in the second cell or in cells surrounding the second cell.

The method of altering the apoptosis may be performed in an in vitro assay. Alternatively, the first cell and the second cell are located within a mammalian organism and the method is performed in vivo.

In the methods of altering SHIV A expression, the inhibition of the expression of SHIV A in the second cell may involve contacting SHIV A produced by the second cell with an agent that binds to and/or inactivates the SHIV A. Alternatively, the inhibition of expression of SHIV A in the second cell comprises contacting the second cell with a nucleic acid construct that reduces the expression of SHIV A in the second cell. In preferred embodiments, the inhibitory agent may be a small molecule inhibitor, or an antibody preparation.

The present invention also is directed to a method of ameliorating inflammatory disease in an individual comprising administering to the individual a composition comprising SHIV A, in an amount effective to deplete B-cells and/or T-cells in the individual. In such a method, the depletion in B-cells and/or T cells is preferably due to apoptosis induced by the SHIV A.

Also disclosed is a transgenic non-human animal, wherein the neuronal cells of the animal comprise a gene that encodes an SHIV A protein, under the control of a neuron-specific promoter. Preferred transgenic non-human animals of the invention exhibit dementia.

Further the present invention contemplates a recombinant host cell, wherein the cell is transformed with an expression construct comprising a nucleic acid that encodes SHIV A under the control of a promoter. Preferably, the cell is a neuronal cell, or a macrophage, but it should be understood that the methods and compositions of the present invention may be employed to prepare any recombinant host cell, e.g., such as a recombinant host cell that may be used for the expression of a protein. In preferred embodiments, the recombinant cell is a mammalian cell that further expresses one or more HIV-related genes selected from the group consisting of tat, nef, rev, vpr, vpu, env, pol, gag, and vpf. Preferably, the recombinant cell is one which has been transformed to express the one or more HIV-related genes. In such embodiments, the expression construct for encoding the SHIV A may further comprise nucleic acids sequences of the one or more HIV-related genes.

The present invention also contemplates methods of treating a subject having HIV-associated dementia comprising administering a composition comprising an isolated polypeptide encoding a SHIV A protein having the sequence of SEQ ID NO:2.

Another aspect of the present invention related to a method of determining the efficacy of an HIV treatment regimen comprising monitoring the expression of SHIV A in the subject receiving the HIV treatment prior to and after the treatment wherein a decrease in the expression of SHIV A after the treatment indicates that the treatment was effective in alleviating the symptoms of HIV infection.

Yet another aspect of the present invention involves a method for screening for agents that modulate apoptosis comprising: providing a cell that expresses SHIV A; contacting the cell with a candidate modulator; and monitoring a change in the expression or activity of SHIV A that occurs in the presence of the modulator. In preferred embodiments, the monitoring step comprises comparing the level of expression of the SHIV A in the presence of the modulator with the level of expression of the SHIV A in the absence of the modulator. More particularly, the monitoring step comprises determining the level of secretion of a 6 kDa fragment of SHIV A in the presence of the modulator with the level of secretion of the 6 kDa fragment of SHIV A in the absence of the modulator. In other embodiments, the monitoring step comprises comparing apoptosis of cells surrounding the cell in the presence of the modulator to the level of apoptosis of surrounding cells in the absence of the cell.

In the screening methods, the cell that expresses the SHIV A is preferably a macrophage or microglial cell and the surrounding cells is a cell selected from the group consisting of a neuronal cell, a B-cell and a T-cell. In certain embodiments, the cell that expresses the SHIV A has been derived from a HIV-infected patient. Alternatively, the cell that expresses the SHIV A is a recombinant host cell engineered to express SHIV A. In certain screening methods the contacting is performed in vitro. In other screening methods, the cell that expresses the SHIV A is located within a mammalian organism and the screening method is performed in vivo. In these latter embodiments, the cell that expresses the SHIV A is preferably part of a transgenic, non-human animal. The candidate modulator may be any compound or agent that alters the expression of SHIV A, such as, for example, a nucleic acid construct that reduces the expression of SHIV A, or an antibody (e.g., a monoclonal antibody). The invention also encompasses compositions comprising modulators identified according to the screening methods of the present invention.

Also disclosed herein are kits for determining the presence of a SHIV A protein in a sample, which comprise a monoclonal antibody of the present invention and a composition comprising an SHIV A protein.

Other features and advantages of the present invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1. SHIV A protein. FIG. 1A. Protein structure of the proapoptotic factors the hypothetical protein SHIV A (SEQ ID NO:2). FIG. 1B. Western blot analysis of the SHIV A protein. Lysate and supernatant from E. coli expressing the SHIV A protein were run on a 10% polyacrylamide gel, transferred onto nitrocellulose paper and blotted with the rabbit polyclonal anti-proapoptotic factor antibody.

FIG. 2. The SHIV A protein induces apoptosis in unstimulated PBMC. Supernatant from E. coli and supernatant from E. coli containing the SHIV A protein were incubated with PBMC for 2 hours and apoptosis determined by Annexin V staining. In blocking experiments either the rabbit polyclonal or murine anti-apoptotic factor antibodies or pre-immune rabbit serum or isotype specific (IgG1) irrelevant murine monoclonal antibodies were added to supernatant containing the SHIV A for 2 hours and apoptosis evaluated by Annexin V staining. The percentage of positively staining cells is indicated in the right upper corner of each panel.

FIG. 3. Purification of the 6000 d peptide. Two liters of E. coli supernatant containing the SHIV A protein was lyophilized and then passed over a DEAE sepharose column. Increasing the concentration of NaCl from 100 mM to 1 M eluted fractions. Only fractions that were western blot positive were run on a 10% polyacrylamide gel and silver stained.

FIG. 4. Supernatant from the FL14676485 transfected Bosc and 43 cells induce apoptosis in target PBMC. Different concentrations (50%, 25%, 10%, and 0%) of supernatant from the FL14676485 and GFP transfected Bosc and 43 cells were co-cultured with freshly isolated PBMC for 2 hours and then assessed for apoptosis by Annexin V staining. The percentage of positively staining cells is indicated in the right upper corner of each panel.

FIG. 5. Western blot analysis of the lysate and supernatant from FL14767485 cDNA transfected and untransfected 43 and Bosc cells and ₄₃HIV cells. Forty-eight hours after transfection of the 43 and Bosc cells, the transfected and untransfected cells along with 43HIV were lysed and the lysates and supernatants run on a 10% polyacrylamide gel and then analyzed by western blot analysis using the rabbit polyclonal anti-apoptotic factor antibodies.

FIG. 6. PCR analysis for FLJ14676485 mRNA in 43 and ₄₃HIV cells. RNA was extracted from 43 and ₄₃HIV cells, reverse transcribed, amplified with an SHIV A or actin-specific primer sets, then the PCR products run on a 1.5% agarose gel. Omitting the RNA from the DNA amplification step performed negative controls.

FIG. 7. Purified populations of CD4+ and CD8+T cells and B cells were isolated by RosetteSepTM, incubated with different concentrations (50%, 25%, 10%, and 0%) of the SHIV A protein for 2 hours and apoptosis determined by Annexin V staining. The percentage of positively staining cells is indicated in the right upper corner of each panel.

FIG. 8. Induction of apoptosis in murine splenocytes. Different concentrations (50%, 25%, 10% and 0%) of supernatant containing the SHIV A protein were added to murine T cell populations and apoptosis evaluated by Annexin V staining. The percentage of positively staining cells is indicated in the right upper corner of each panel.

FIG. 9. Detection of PARP fragments and activation of caspase 3 in SH-SY5Y cells. SH-SY5Y cells were incubated with different concentrations of the SHIV A protein (50%, 25%, 10%, 1%, 0%) for 5 hours and the cells lyzed and prepared for western blot analysis with Abs directed against the 85-kDa PARP fragment and activated 17 kDa caspase 3 fragments. The lysate was run on a 10% polyacrylamide gel, transferred onto nitrocellulose and then blotted with anti-PARP and anti-caspase 3 antibodies.

FIG. 10. Detection of the pro-apoptotic factor in patients with HAD. Immunofluorescence staining was performed using tissue sections from normal brain, HAD, Alzheimer's disease, and non-HIV-1 encephalitis. The sections were stained with murine FITC labeled anti-SHIV A antibodies and analyzed by confocal microscopy. Two observers routinely observed 10 separate fields.

FIG. 11. Detection of the SHIV A protein from lymph nodes. Immunofluoresence was also performed on sections of lymph nodes from the same patients in FIG. 10. The sections were stained with FITC-labeled murine anti-proapoptotic factor antibody and analyzed by confocal microscopy. Two observers routinely observed 10 separately fields.

FIG. 12 Intracytoplasmic staining for SHIV A and p24. 43_{HIV} cells were infected with HIV-1_{BaL} (FIG. 12A), HIV-1_{89.6} (FIG. 12B) and HIV-1 obtained from 43_{HIV} cells after 5 weeks of infection (FIG. 12C) and then stained intracytoplamically at weekly intervals for the presence of p24 and SHIV A. For intracytoplasmic staining, 43_{HIV} cells at 1, 2, 3, 4 and 5 weeks after infection were fixed and permeabilized with 70% ethanol for 30 minutes at 4° C. The cells were then washed three times with PBS and phycoerythrin labeled anti-p24 antibodies, FITC labeled anti-SHIV A antibodies and isotype matched controls antibodies were added for 30 minutes at 4° C. The cells were then washed 3 times in PBS and analyzed by flow cytometry. The percentage of positively staining cells is indicated in the right upper corner. This is representative of an experiment repeated 3 times.

FIG. 13. Relative copy number of SHIV A mRNA in 43 cells after HIV-1 infection. Forty-three cells were infected with HIV-1_{BaL}, HIV-1_{89.6} and HIV-1 isolated 5 weeks after infection and mRNA extracted at different time points (1, 2, 3, 4 and 5 weeks) after infection. Real time PCR for SHIV A was performed in a BioRad Cycler. Data are expressed as relative copy number of SHIV A. This is representative of an experiment repeated 3 times.

FIG. 14. Northern blot for SHIV A. We probed multiple human tissues by northern blot including heart, brain, placenta, lung, liver, skeletal muscle, kidney, pancreas, testes, ovary, small intestine, colon, peripheral blood leukocytes, lymph nodes, bone marrow, fetal liver and thymus on a Master Blot using a DNA probe from the FL14676485 gene that encodes SHIV A. This is representative of an experiment repeated 3 times.

FIG. 15. SHIV A fusion protein. A SHIV A fusion protein made from AA 330 to 660 of the full length protein that had pro-apoptotic activity was run on a 12.5% SDS polyacrylamide gel and stained with 0.1% Commassie Blue. This is representative of an experiment repeated 5 times.

FIG. 16. Pro-apoptotic activity of SHIV A on the THB and H-9 T cell lines and primary T cells. FIG. 16A Different concentrations (0.1, 1, 10 and 100 μg/ml) of the SHIV A fusion protein were used to treat the THB and H-9 cell lines and the 2 primary T cell preparations for 3 hours. FIG. 16B. Apoptosis was determined using FITC-labeled Annexin V. The cells were analyzed by flow cytometry and the percentage of positively staining cells is indicated in the right upper corner. This is representative of an experiment repeated 5 times.

FIG. 17. Induction of apoptosis in neuronal tissue. FIG. 17A. The SH-SY5Y, IMR, and MC-IXC cell lines and 2 preparations of primary neurons were treated with 1 μg/ml of the SHIV A fusion protein for 3 hours and apoptosis determined by Annexin V staining. The percentage of positively staining cells is indicated in the right upper corner. This is representative of an experiment repeated 3 times. FIG. 17B. Increased apoptosis induced by SHIV A in neurons. We used different concentrations of the SHIV A fusion protein (0, 0.1. 1, and 10 μg/ml) to assess apoptotic activity in primary neurons as detected by intracytoplasmic staining with FITC-labeled antibodies directed against activated caspase 3. The mean fluorescence intensity of Caspase 3 staining (y axis) was plotted against the different concentrations of the SHIV A fusion protein (x axis) used to treat the neurons. This is representative of an experiment repeated 3 times. FIG. 17C. Determination of apoptosis by ELISA. Cells (SH-SY5Y, IMR, MC-IXC, THB and H-9) were treated with different concentrations (0.01, 0.1, 1, 10 and 100 μg/ml) of SHIV A for 16 hours followed by the labeling of active caspases with biotin-ZVKD-fmk. The cells were then lyzed, and active Caspase-3 measured by Ag capture ELISA. Standard curves containing known concentrations of activated Caspase-3 were generated for each assay. This is representative of an experiment repeated 3 times.

FIGS. 18A and 18B. Apoptotic signaling events I. FIG. 18A. Caspase usage. The SH-SY5Y, IMR, and MC-IXC neuronal cell lines and primary neurons were treated with 1 μg/ml of the SHIV A fusion protein for 16 hours, lyzed, run on a 12.5% polyacrylamide gel, transferred onto nitrocellulose paper and subjected to western blot analysis using anti-Caspase 8 and Caspase 9 antibodies that recognize active apoptotic fragments. This is representative of an experiment repeated 3 times. FIG. 18B. Release of cytochrome c into the cytoplasm. The SH-SY5Y, IMR, and MC-IXC cell lines and the primary neurons were treated with the SHIV A fusion protein (1 μg/ml); the mitochondria and cytosolic fractions were extracted, run on a 12.5% polyacrylamide gel, transferred onto nitrocellulose membranes and subjected to western blot analysis using rabbit polyclonal cytochrome c specific antibodies. This is representative of an experiment repeated 3 times.

FIG. 19. Apoptotic Signaling events II. FIGS. 19A and 19B. Activation of Bax and Bad and suppression of Bcl-2 and Bcl-xL in SH-SY5Y and THB cells. SH-SY5Y and THB cells were treated with SHIV A for 16 hours or left untreated, lyzed and western blot performed using antibodies against non-activated Apaf-1, Bad, Bax, Bcl-2, Bcl-xL, Bruce, CAS, hILP/XIAP, Mcl-1, Nip1 and p53 proteins. Only the results with Bax, Bad, Bcl-2 and Bcl-xL are presented. This is represented of an experiment repeated 3 times. FIG. 19C. SH-SY5Y and THB cells were transfected with Bcl-2, followed by intracytoplasmic staining with anti-Bcl-2 antibodies to assess the efficiency of the transfection (upper panels). Bcl-2 and untransfected SH-SY5Y cells were treated with SHIV A for 16 hours and apoptosis was measured by Caspase 3 ELISA. This is representative of an experiment repeated 3 times. FIG. 19D. Effect of MAP kinase inhibitor SB203580 on SHIV A induced apoptosis. The SH-SY 5Y, IMR, MC-IXC, H-9 and THB cells were treated with different concentrations (0.0001-10 μg/ml) of the MAP kinase inhibitor SB203580 for 1 hour and then exposed to 100 μg/ml of SHIV A for 16 hours. Apoptosis was determined by Caspase 3 ELISA. This is representative of an experiment repeated 3 times.

FIG. 20. Nitric oxide and intracellular glutathione production. FIG. 20A. Nitric Oxide production. SH-SY5Y, IMR, MC-IXC, H-9, and THB cells were treated with different concentrations (10⁻³-100) of SHIV A for 16 hours. The supernatants were harvested and analyzed by the Greiss reaction. Mean fluorescence intensity was read at an absorbance between 520-560 nm. Standard curves were established for each assay. This is representative of an experiment repeated 3 times. FIG. 20B. Glutathione production. SH-SY5Y, IMR, MC-IXC, H-9, and THB cells were treated with different concentrations (10⁻³-100) of SHIV A for 16 hours, followed by centrifugation; the cells were then suspended in ice-cold PBS lysis buffer before addition of monochlorobimane and glutathione-S-transferase for 30 minutes at 37° C. Standard curves were established for each assay. Mean fluorescence intensity was read in a plate reader at 380/460 nm. This is representative of an experiment repeated 3 times. FIG. 20C. Different concentrations of NAC (10⁻⁴-10⁻¹M) were added to SH-SY5Y, IMR, MC-IXC, H-9 and THB cells for 1 hour prior to the addition of 100 μg/ml of SHIV A for 16 hours. Apoptosis was determined by Caspase 3 ELISA.

FIG. 21. Possible role of SHIV A in HIV-related neuronal damage. The main pathway of HIV-1 entry into the brain occurs by means of infected CD14^{low}CD16^{high} macrophages, the phenotype of clone 43. Once in the brain, infected CD14^{low}CD16^{high} macrophages release viral envelope proteins (gp120), cytokines (TNF-χ) and chemokines, which in turn activate uninfected macrophages and microglia. Immune activated- and HIV-infected brain macrophages also release other potentially neurotoxic substances including quinolinic acid and EAAs such as glutamate and L-cysteine, arachidonic acid, PAF, NTox, free radicals, TNF-α and SHIV A. These substances induce neuronal injury, dendritic and synaptic damage, and apoptosis. The 43_{HIV} cells are a source of gp120 and CXCR4 and CCR5 using HIV species that can produce induce apoptosis in neurons and astrocytes that express CCR5 and CXCR4 receptors. Macrophages and astrocytes have mutual feedback loops (reciprocal arrows). These cytokines stimulate astrocytosis. Neuronal injury is mediated predominantly by over-activation of NMDAR-coupled ion channels that allow excessive influx of Ca²⁺ that may be the way SHIV A induces neuronal apoptosis. SHIV A may act directly as a neurotoxin to induce neuronal apoptosis or act additively with TNF-α (also produced by 43_{HIV} cells after HIV infection).

### DETAILED DESCRIPTION OF THE PREFERRED EMBODIMENTS

The prognosis for HIV-infected patients has improved markedly with the introduction of HAART. However, even in the absence of detectable HIV viral replication and/or load, the expression of HIV-induced proteins in a previously infected patient results in secondary diseases. One such secondary disease, HAD, is increasingly a cause of significant HIV-associated morbidity. Even though HAART has resulted in a decrease in the incidence of HAD (Dore et al., J. Acquir. Immune. Defic. Syndr. Hum. Retrovirol. 16, 39-43, Ferrando et al., AIDS 12:F65-F70, 1998), such therapy fails to provide complete protection from, or reversal of, HAD (Dore et al., AIDS 13:1249-1253, 1999, Major et al., Science 288: 440-442, 2000). In addition, the prevalence of the dementia may eventually increase as people live longer with AIDS (Lipton et al., N. Engl. J. Med. 332:934-940, 1995, Gartner, Science 287:602-604, 2000). Currently there is no specific treatment for HAD, mainly because of an incomplete understanding of how HIV infection causes neuronal injury and apoptosis.

The present invention identifies a factor released from macrophages upon HIV-1 infection. This factor is secreted from the macrophages and induces apoptosis in surrounding cells. More particularly, the present invention teaches that there is a novel cDNA clone isolated from the chronically HIV-1 infected human macrophage cell line, ₄₃HIV that induces apoptosis in T cells and B cells, and in neuronal cells. In certain embodiments, induction in the 43 cells was determined by intracytoplasmic staining and real time RNA PCR four weeks after HIV infection. The cDNA was isolated using an antibody-based screen of an expression cDNA library from ₄₃HIV cells, wherein these monoclonal antibodies were originally raised against the active 6 kDa apoptosis factor purified from ₄₃HIV supernatants. Furthermore, it is demonstrated herein that the novel cDNA clone encodes a proapoptotic factor which is present in brain and lymphoid tissue from patients that are HIV-1 infected and exhibit HAD but not in non-HIV-1 infected controls, Alzheimer's patients, and non-HIV-1 encephalitis patients. Methods and compositions for exploiting this discovery are discussed in further detail herein below.

During the course of 43 cell infection with HIV_{BaL}, there was a change in co-receptor usage of progeny viruses produced from strictly CCR5 co-receptor usage to both CCR5 and CXCR4 co-receptor usage. However, infection with dual tropic HIV-1 isolates (HIV89.6 and HIV from 43HIV 4 weeks after infection) did not result in more rapid production of SHIV A mRNA for SHIV A was present in the thymus and lymph nodes. Using a biologically active fusion protein corresponding to amino acids 330-660 of SHIV A the inventors demonstrated that it is more potent in inducing apoptosis in primary neurons and neuronal cell lines than it is in primary T cells and T cell lines. SHIV A causes apoptosis by inducing NO and the secretion of glutathione, activating Bax and Bad and suppressing Bcl-2 and Bcl-xL causing the release of cytochrome c from the mitochondria that activate Caspase 9. Transfected Bcl-2, the anti-oxidant N-acetyl-cysteine and the NMDA receptor antagonist memantine block SHIV A induced apoptosis.

The present inventors have shown that SHIV A protein is a 66 kDa full length protein of SEQ ID NO:2. This protein has proapoptotic activity. The proapoptotic activity is associated with a 6 kDa protein secreted from macrophages upon infection by HIV-1 and also from bacteria expressing the SHIV A cDNA. The present invention contemplates polynucleotides encoding this factor and the use of these compositions or antagonists thereof for the diagnosis, prevention and intervention of apoptosis of HIV-infected cells, especially neuronal cells, T cells and macrophages of individuals infected with HIV-1. Additionally, the SHIV A protein compositions and methods may be used to augment or increase apoptosis in disorders that involve T cells and B-cell, e.g., inflammation, auto immune disorders, respiratory distress syndromes, and infection. It may also be desirable to increase apoptosis in for example cancer cells. Expressing or augmenting the expression of SHIV A protein, in such cancer cells will be useful in promoting such apoptosis.

A. Polypeptide and Fragments Thereof.

According to the present invention, there has been identified a gene encoding a SHIV A protein, a protein whose expression and/or secretion from macrophages is increased as a result of HIV-1 infection. This increased expression and/or secretion causes apoptosis in neuronal cells in culture, which ultimately leads to the physiological phenotypes manifest in HAD and other manifestations of HIV-induced apoptosis such as, but not limited to CD4 lymphocytopenia. It is contemplated that inhibition of the expression of this protein will have a beneficial effect in treating HAD and other HIV-associated apoptosis-mediated diseases. The inventors further showed that, in addition to neuronal cells, this protein is expressed in T-cells and B-cells. In certain embodiments, it is contemplated that it will be desirable to increase the expression of SHIV A protein in T-cells and B-cells in disorders which result from an aberrant accumulation of these cells e.g., inflammatory diseases, autoimmune diseases, and the like.

An additional embodiment in which it would be desirable for increase, augment or otherwise supplement endogenous SHIV A protein expression and/or activity is in situations where cell death would be desirable. For example, such a result would be desirable in combating hyperproliferative disorders, cancers and neoplasia among others. Such methods of increasing, augmenting or supplementing endogenous activity may involve supplying to a cell or an organism a composition comprising an isolated polypeptide encoding a SHIV A protein and an immunological adjuvant, or pharmaceutically acceptable carrier or diluent. Such protein-based compositions are discussed in further detail herein below.

Human SHIV A protein has been cloned by the present inventors and is taught herein to be encoded by a nucleic acid sequence as shown in SEQ ID NO:1. The coding region of the FL14676485 gene encodes a SHIV A protein of SEQ ID NO:2.

In addition to the entire SHIV A protein molecule of SEQ ID NO:2, the compositions of the present invention also may employ fragments of the polypeptide that may or may not retain the biological activity of SHIV A protein. Fragments, including the N-terminus or C terminus of the molecule may be generated by genetic engineering of translation start or stop sites within the coding region (discussed below). Alternatively, treatment of the SHIV A protein molecule with proteolytic enzymes, known as proteases, can produce a variety of N-terminal, C-terminal and internal fragments. Examples of fragments may include contiguous residues of the SHIV A protein sequence of SEQ ID NO:2, of 6, 7; 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, or more amino acids in length. Such fragments preferably retain one or more of the biological activities of SHIV A protein and/or retain an immunological (antigenic) property of SHIV A protein. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration). A particularly preferred fragment of the protein of SEQ ID NO:2 is one which comprises amino acids 330 to 660 of SEQ ID NO:2. This fragment is demonstrated herein as having apoptotic activity. Other fragments of the SHIV A protein may readily be generated by those of skill in the art and will be expected to have apoptotic activity. Such fragments include fragments from amino acids 310 to 660, 300 to 660, 290 to 660, 280 to 660, 270 to 660, 266 to 660, 250 to 660 and other fragments that contain some or all of amino acids 330 to 660. Thus, the amino terminus of the fragment may end at amino acid 10, 20, 30, 40, 50, 60, 70, 80, 90, 100 110, 120, 130, 140, 150, 160, 170, 180, 190, 200, 210, 220, 230, 240, 250, 260, 270, 280, 290, 300, 310, 320, 340, 350, 360 or any amino acid between any of two of these residues. It is expected that one of skill would readily generate such fragments of the invention by, e.g., serially adding amino acids (one at a time or more than one at a time) to the N-terminal end of a fragment of amino acids 330 to 660 and test each of the generated fragments in any apoptosis or other assay and compare that fragments effects to those observed in such an assay by the action of the 330 to 660 fragment or indeed the full-length SHIV A protein.

Another preferred fragment is one which comprises between about amino acid 330 and 450 of SHIV A of SEQ ID NO:2. Those of skill in the art will be able to generate peptides of SEQ ID NO:2 in which the carboxy terminus is gradually decreased and the activity of such fragment monitored for SHIV A-like activity. Thus the carboxy terminus of the fragment may thus end at 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660 or any amino acid between any of these residues. Again it is expected that one of skill would readily generate such fragments of the invention by, e.g., serially adding amino acids (one at a time or more than one at a time) to the C-terminal end of a fragment ending at e.g., 360 of SHIV A and test each of the generated fragments in any apoptosis or other assay and compare that fragments effects to those observed in such an assay by the action of the 330 to 660 fragment or indeed the full-length SHIV A protein.

a. Structural Features of the Polypeptide.

The FL14676485 gene encodes SHIV A protein having a molecular weight of 66 kDa. This cDNA has 8 open reading frames encoding for proteins with molecular weights of 9900 d, 6490 d, 4400 d, 4290 d, 4070 d, 4100 d, 3960 d, and 3740 d (NCBI Open Reading Frame Finder). The SHIV A protein is cleaved to a 6 kDa proapoptotic factor mature protein, which is secreted from ₄₃HIV cells. Thus at a minimum, this molecule may be used as standards in assays as a molecular weight marker. Moreover, the 6 kDa protein is shown herein to possess a proapoptotic activity, and may therefore be used as a marker for apoptosis. Active fragments of the SHIV A protein are contemplated to be useful in the various methods of the present invention. A particularly preferred active fragment of the present invention is a protein comprising amino acids 330 to 661 of the full length SHIV A protein. This protein is denoted herein as SEQ ID NO:3.

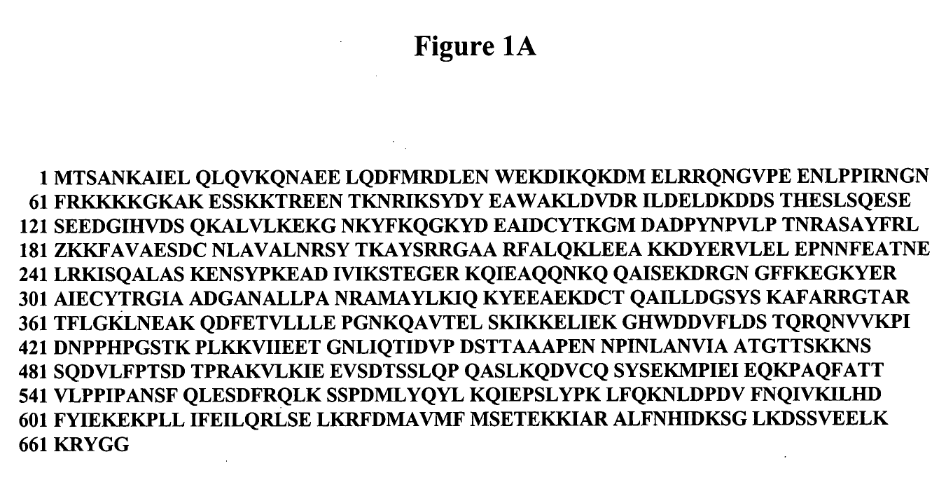

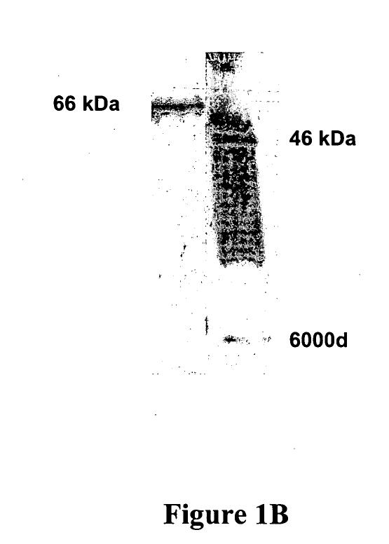

In addition, motif analysis of the SHIV A protein revealed 2 tetratricopeptide (TPR) repeats the TPR repeat is a repeat structure of 34 amino acids first described in yeast and later found to occur in a large number of proteins (FIG. 1a). A common feature of TRP repeats is protein-protein interactions (Lamb et al., Trends in Biochem Sci 20:257, 1995). It has also been proposed that TPR proteins preferentially interact with WD-40 repeat proteins but in many instances TPR aggregate to form multi-protein complexes (Das and Cohen, EMBO J. 17:11192, 1998). TPR repeats have been implicated in apoptosis (Demonacos et al., Molecular Cell 8:71).

In characterizing the proapoptotic factor of the present invention, the inventors determined, by molecular weight fractionation, that pro-apoptotic activity was present from the fractions that corresponded to a molecular weight less than 10,000 Da (Chen et al., J Immunology 161:4257, 1998). Furthermore, fractionation of supernatants from HIV-1BAL infected monocytes that induced apoptosis in target PBMC revealed that activity was also present in those fractions with a molecular weight less than 10 Da similar to the ₄₃HIV cell line (Chen et al., J Immunology 161:4257, 1998).

b. Functional Aspects.

When the present application refers to the function of SHIV A protein or “wild-type” activity, it is meant that the molecule in question has the ability to induce apoptosis in neuronal cells, T cells and B cells. Other activities that are attributable to the normal SHIV A protein product may include protein-protein interactions typical of TRP repeat containing proteins. An assessment of the particular molecules that possess such activities may be achieved using standard assays familiar to those of skill in the art.

In certain embodiments, SHIV A protein analogs and variants may be prepared and will be useful in a variety of applications. Amino acid sequence variants of the polypeptide can be substitutional, insertional or deletion variants. Deletion variants lack one or more residues of the native protein which are not essential for function or immunogenic activity. A common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, also called fusion proteins, are discussed below.

Substitutional variants typically exchange one amino acid of the wild type for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

A particular aspect of the present invention contemplates generating SHIV A protein mutants in which the TRP repeats are mutated. Such mutants will yield important information pertaining to the biological activity, physical structure and receptor or ligand binding potential of the SHIV A protein molecule. An alternative approach employs alanine scanning in which residues throughout molecule are randomly replaced with an alanine residue.

In order to construct such mutants, one of skill in the art may employ well known standard technologies. Specifically contemplated are N-terminal deletions, C-terminal deletions, internal deletions, as well as random and point mutagenesis.

N-terminal and C-terminal deletions are forms of deletion mutagenesis that take advantage for example, of the presence of a suitable single restriction site near the end of the C- or N-terminal region. The DNA is cleaved at the site and the cut ends are degraded by nucleases such as BAL31, exonuclease III, DNase I, and S1 nuclease. Rejoining the two ends produces a series of DNAs with deletions of varying size around the restriction site. Proteins expressed from such mutants can be assayed for appropriate apoptotic activity as described throughout the specification. Similar techniques may be employed for internal deletion mutants by using two suitably placed restriction sites, thereby allowing a precisely defined deletion to be made, and the ends to be religated as above.

Also contemplated are partial digestion mutants. In such instances, one of skill in the art would employ a “frequent cutter”, which cuts the DNA in numerous places depending on the length of reaction time. Thus, by varying the reaction conditions it will be possible to generate a series of mutants of varying size, which may then be screened for activity.

A random insertional mutation may also be performed by cutting the DNA sequence with a DNase I, for example, and inserting a stretch of nucleotides that encode, 3, 6, 9, 12 etc., amino acids and religating the end. Once such a mutation is made the mutants can be screened for various activities presented by the wild-type protein.

Point mutagenesis also may be employed to identify with particularity which amino acid residues are important in particular activities associated with SHIV A protein. Thus, one of skill in the art will be able to generate single base changes in the DNA strand to result in an altered codon and a missense mutation.

The amino acids of a particular protein can be altered to create an equivalent, or even an improved, second-generation molecule. Such alterations contemplate substitution of a given amino acid of the protein without appreciable loss of interactive binding capacity with structures such as, for example, antigen-binding regions of antibodies or binding sites on substrate molecules or receptors. Since it is the interactive capacity and nature of a protein that defines that protein's biological functional activity, certain amino acid substitutions can be made in a protein sequence, and its underlying DNA coding sequence, and nevertheless obtain a protein with like properties. Thus, various changes can be made in the DNA sequences of genes without appreciable loss of their biological utility or activity, as discussed below. Table 1 below shows the codons that encode particular amino acids.

In making such changes, the hydropathic index of amino acids may be considered. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte & Doolittle, J. Mol. Biol., 157(1):105-132, 1982, incorporated herein by reference). Generally, amino acids may be substituted by other amino acids that have a similar hydropathic index or score and still result in a protein with similar biological activity, i.e., still obtain a biological functionally equivalent protein.

In addition, the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As such, an amino acid can be substituted for another having a similar hydrophilicity value and still obtain a biologically equivalent and immunologically equivalent protein.

Exemplary amino acid substitutions that may be used in this context of the invention include but are not limited to exchanging arginine and lysine; glutamate and aspartate; serine and threonine; glutamine and asparagine; and valine, leucine and isoleucine. Other such substitutions that take into account the need for retention of some or all of the biological activity whilst altering the secondary structure of the protein will be well known to those of skill in the art.

Another type of variant that is specifically contemplated for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of SHIV A protein, but with altered and even improved characteristics.

Other mutants that are contemplated are those in which entire domains of the SHIV A protein are switched with those of another related protein. Domain switching is well-known to those of skill in the art and is particularly useful in generating mutants having domains from related species.

Domain switching involves the generation of chimeric molecules using different but related polypeptides. For example, by comparing the sequence of SHIV A protein with that of similar sequences from another source and with mutants and allelic variants of these polypeptides, one can make predictions as to the functionally significant regions of these molecules. It is possible, then, to switch related domains of these molecules in an effort to determine the criticality of these regions to SHIV A protein function. These molecules may have additional value in that these “chimeras” can be distinguished from natural molecules, while possibly providing the same or even enhanced function.

In addition to the mutations described above, the present invention further contemplates the generation of a specialized kind of insertional variant known as a fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. It is likely that the SHIV A protein is a secreted chemokine, which has a receptor on neuronal cells, T-cells and/or B cells. Fusion to a polypeptide that can be used for purification of the receptor-SHIV A protein complex would serve to isolate the receptor for identification and analysis.

There are various commercially available fusion protein expression systems that may be used in the present invention. Particularly useful systems include but are not limited to the glutathione S-transferase (GST) system (Pharmacia, Piscataway, N.J.), the maltose binding protein system (NEB, Beverley, Mass.), the FLAG system (IBI, New Haven, Conn.), and the 6xHis system (Qiagen, Chatsworth, Calif.). These systems are capable of producing recombinant polypeptides bearing only a small number of additional amino acids, which are unlikely to affect the antigenic ability of the recombinant polypeptide. For example, both the FLAG system and the 6xHis system add only short sequences, both of which are known to be poorly antigenic and which do not adversely affect folding of the polypeptide to its native conformation. Another N terminal fusion that is contemplated to be useful is the fusion of a Met Lys dipeptide at the N terminal region of the protein or peptides. Such a fusion may produce beneficial increases in protein expression or activity.

A particularly useful fusion construct may be one in which a SHIV A protein or peptide is fused to a hapten to enhance immunogenicity of a SHIV A protein fusion construct. Such fusion constructs to increase immunogenicity are well known to those of skill in the art, for example, a fusion of SHIV A protein with a helper antigen such as hsp70 or peptide sequences such as from Diptheria toxin chain or a cytokine such as IL-2 will be useful in eliciting an immune response. In other embodiments, fusion construct can be made which will enhance the targeting of the SHIV A protein related compositions to a specific site or cell.

Other fusion constructs including a heterologous polypeptide with desired properties, e.g., an Ig constant region to prolong serum half life or an antibody or fragment thereof for targeting also are contemplated. Other fusion systems produce polypeptide hybrids where it is desirable to excise the fusion partner from the desired polypeptide. In one embodiment, the fusion partner is linked to the recombinant SHIV A protein polypeptide by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

It will be desirable to purify SHIV A protein or variants thereof; Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing; affinity columns specific for protein fusion moieties; affinity columns containing SHIV A-specific antibodies. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al. Biochem. Biophys. Res. Comm., 76:425, 1977). It will therefore be appreciated that under differing electrophoresis conditions, the apparent molecular weights of purified or partially purified expression products may vary.

In addition to the full length SHIV A protein described herein, smaller SHIV A protein-related peptides may be useful in various embodiments of the present invention. Such peptides or indeed even the full length protein, of the invention can also be synthesized in solution or on a solid support in accordance with conventional techniques. Various automatic synthesizers are commercially available and can be used in accordance with known protocols. See, for example, Stewart and Young, Solid Phase Peptide Synthesis, 2nd. ed., Pierce Chemical Co., (1984); Tam et al., J. Am. Chem. Soc., 105:6442, (1983); Merrifield, Science, 232: 341-347, (1986); and Barany and Merrifield, The Peptides, Gross and Meienhofer, eds, Academic Press, New York, 1-284, (1979), each incorporated herein by reference. The SHIV A protein active protein or portions of the SHIV A protein, which correspond to the selected regions described herein, can be readily synthesized and then screened in screening assays designed to identify reactive peptides.

Alternatively, recombinant DNA technology may be employed wherein a nucleotide sequence which encodes a peptide of the invention is inserted into an expression vector, transformed or transfected into an appropriate host cell and cultivated under conditions suitable for expression as described herein below.

U.S. Pat. No. 4,554,101 (incorporated herein by reference) also teaches the identification and preparation of epitopes from primary amino acid sequences on the basis of hydrophilicity. Thus, one of skill in, the art would be able to identify epitopes from within any amino acid sequence encoded by any of the DNA sequences disclosed herein.

As discussed herein below, the SHIV A proteins or peptides may be useful as antigens for the immunization of animals relating to the production of antibodies. It is envisioned that either SHIV A protein, or portions thereof, may be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

B. SHIV A-related Nucleic Acids

The present invention also provides, in another embodiment, an isolated nucleic acid encoding SHIV A protein. The nucleic acid or gene for the human SHIV A protein molecule has been identified. Preferred embodiments of the present invention are directed to nucleic acid constructs comprising a polynucleotide that encodes the human SHIV A protein, operably linked to a heterologous promoter The present invention is not limited in scope to the particular gene(s) identified herein, however, seeing as one of ordinary skill in the art could, using the nucleic acids corresponding to the FL14676485 gene, readily identify related homologs in various other species (e.g., rat, rabbit, monkey, gibbon, chimp, ape, baboon, cow, pig, horse, sheep, cat and other species).

In addition, it should be clear that the present invention is not limited to the specific nucleic acids disclosed herein. As discussed below, a “FL14676485 gene” may contain a variety of different nucleic acid bases and yet still produce a corresponding polypeptide that is functionally indistinguishable, and in some cases structurally, from the human gene disclosed herein. The term “FL14676485 gene” may be used to refer to any nucleic acid that encodes a SHIV A protein, peptide or polypeptide and, as such, is intended to encompass both genomic DNA and cDNA.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of SHIV A protein, its receptor or endogenous protein on which SHIV A has an effect.

a. Nucleic Acids Encoding SHIV A Protein.

The human gene that encodes SHIV A protein is disclosed in SEQ ID NO:1. Nucleic acids according to the present invention (which include genomic DNA, cDNA, mRNA, as well as recombinant and synthetic sequences and partially synthetic sequences) may encode an entire SHIV A protein, polypeptide, or allelic variant, a domain of SHIV A protein that expresses a proapoptotic activity, or any other fragment or variant of the SHIV A protein sequences set forth herein.

The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that due to the redundancy of the genetic code, a given SHIV A protein encoding gene from a given species may be represented by degenerate variants that have slightly different nucleic acid sequences but, nonetheless, encode the same protein (see Table 1 below).

As used in this application, the term “a nucleic acid encoding a SHIV A protein” refers to a nucleic acid molecule that has been isolated from total cellular nucleic acid. In preferred embodiments, the invention concerns a nucleic acid sequence essentially as set forth in SEQ ID NO:1. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Nucleotide sequences that have at least about 50%, usually at least about 60%, more usually about 70%, most usually about 80%, preferably at least about 90% and most preferably about 95% of nucleotides that are identical to the nucleotides of SEQ ID NO:1 are nucleic acids encoding a SHIV A protein. Sequences that are essentially the same as those set forth in SEQ ID NO:1 may also be functionally defined as sequences that are capable of hybridizing to a nucleic acid segment containing the complement of SEQ ID NO:1 under standard conditions.

The DNA segments of the present invention include those encoding biologically functional equivalent SHIV A proteins and peptides as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through any means described herein or known to those of skill in the art.

b. Oligonucleotide Probes and Primers.

Naturally, the present invention also encompasses DNA segments that are complementary, or essentially complementary, to the sequence set forth in SEQ ID NO: 1. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 under relatively stringent conditions such as those described herein. Such sequences may encode the entire SHIV A protein or functional or non-functional fragments thereof.

Alternatively, the hybridizing segments may be shorter oligonucleotides. Sequences of about 17 bases long should occur only once in the human genome and, therefore, suffice to specify a unique target sequence. Nucleotide sequences of this size that specifically hybridize to SEQ ID NO:1 are useful as probes or primers. As used herein, an oligonucleotide that “specifically hybridizes” to SEQ ID NO:1 means that hybridization under suitably (e.g., high) stringent conditions allows discrimination of SEQ ID NO:1 from other apoptotic genes. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, or 1000 bases and longer are contemplated as well. Such oligonucleotides will find use, for example, as probes in Southern and Northern blots and as primers in amplification reactions.

Suitable hybridization conditions will be well known to those of skill in the art. In certain applications, it is appreciated that lower stringency conditions may be required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

One method of using probes and primers of the present invention is in the search for genes related to SHIV A protein, more particularly, homologs of the proteins from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids also are routinely employed in site directed mutagenesis, which eliminates the step of transferring the gene of interest from a phage to a plasmid.

In general, site-directed mutagenesis is performed by first obtaining a single-stranded vector, or melting of two strands of a double, stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Of course site-directed mutagenesis is not the only method of generating potentially useful mutant SHIV A protein species and as such is not meant to be limiting. The present invention also contemplates other methods of achieving mutagenesis such as for example, treating the recombinant vectors carrying the gene of interest mutagenic agents, such as hydroxylamine, to obtain sequence variants.

c. Inhibitory Nucleic Acid Constructs.

As discussed herein, the SHIV A protein is a proapoptotic factor that is secreted by macrophages upon HIV infection. This secreted product causes HAD by inducing apoptosis of neuronal cells. In addition, this factor is involved in systemic HIV and causes apoptosis of T-cells and B-cells. It would be advantageous to disrupt the apoptotic activity of this factor. Such disruption may be achieved using a variety of methods known to those of skill in the art. The present section discusses nucleic acid-based methods of disrupting the activity of SHIV A. For example, the nucleic acid-based techniques may be used to block the expression of SHIV A protein, and therefore, to perturb the SHIV A protein-induced apoptosis. Polynucleotide products which are useful in this endeavor include antisense polynucleotides, ribozymes, RNAi, and triple helix polynucleotides that modulate the expression of SHIV A protein.

Antisense polynucleotides and ribozymes are well known to those of skill in the art. Crooke and B. Lebleu, eds. Antisense Research and Applications (1993) CRC Press; and Antisense RNA and DNA (1988) D. A. Melton, Ed. Cold Spring Harbor Laboratory Cold Spring Harbor, N.Y. Anti-sense RNA and DNA molecules act to directly block the translation of mRNA by binding to targeted mRNA and preventing protein translation. An example of an antisense polynucleotide is an oligodeoxyribonucleotide derived from the translation initiation site, e.g., between −10 and +10 regions of the relevant nucleotide sequence.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozymes) could be designed. These molecules, though having less than 50% homology, would bind to target sequences under appropriate conditions.

It may be advantageous to combine portions of genomic DNA with cDNA or synthetic-sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

As indicated above, the DNA and protein sequences for SHIV A protein is published and disclosed as an EST in Genbank Accession No. NM_{—}024604. Those of skill in the art are referred to the Genbank Database at www.ncbi.nlm.nih.gov, which list this sequence. Related SHIV A protein and/or nucleic acid sequences from other sources may be identified using probes directed at the sequences of SEQ ID NO:1. Such additional sequences may be useful in certain aspects of the present invention. Although antisense sequences may be full length genomic or cDNA copies, they also may be shorter fragments or oligonucleotides e.g., polynucleotides of 100 or less bases. Although shorter oligomers (8-20) are easier to make and more easily permeable in vivo, other factors also are involved in determining the specificity of base pairing. For example, the binding affinity and sequence specificity of an oligonucleotide to its complementary target increases with increasing length. It is contemplated that oligonucleotides of 8, 9, 10, 11, 12, 13, 14, 15, 16, 17, 18, 19, 20, 25, 30, 35, 40, 45, 50, or more base pairs will be used.

Ribozymes are enzymatic RNA molecules capable of catalyzing the specific cleavage of RNA. The mechanism of ribozyme action involves sequence specific interaction of the ribozyme molecule to complementary target RNA, followed by an endonucleolytic cleavage. Within the scope of the invention are engineered hammerhead or other motif ribozyme molecules that specifically and efficiently catalyze endonucleolytic cleavage of RNA sequences encoding protein complex components.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using ribonuclease protection assays. See, Draper PCT WO 93/23569; and U.S. Pat. No. 5,093,246.

Nucleic acid molecules used in triple helix formation for the inhibition of transcription are generally single stranded and composed of deoxyribonucleotides. The base composition must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Another technique that is of note for reducing or disrupting the expression of a gene is RNA interference (RNAi). The term “RNA interference” was first used by researchers studying C. elegans and describes a technique by which post-transcriptional gene silencing (PTGS) is induced by the direct introduction of double stranded RNA (dsRNA: a mixture of both sense and antisense strands). Injection of dsRNA into C. elegans resulted in much more efficient silencing than injection of either the sense or the antisense strands alone (Fire et al., Nature 391:806-811, 1998). Just a few molecules of dsRNA per cell is sufficient to completely silence the expression of the homologous gene. Furthermore, injection of dsRNA caused gene silencing in the first generation offspring of the C. elegans indicating that the gene silencing is inheritable (Fire et al., Nature 391:806-811, 1998). Current models of PTGS indicate that short stretches of interfering dsRNAs (21-23 nucleotides; siRNA also known as “guide RNAs”) mediate PTGS. siRNAs are apparently produced by cleavage of dsRNA introduced directly or via a transgene or virus. These siRNAs may be amplified by an RNA-dependent RNA polymerase (RdRP) and are incorporated into the RNA-induced silencing complex (RISC), guiding the complex to the homologous endogenous mRNA, where the complex cleaves the transcript.

While most of the initial studies were performed in C. elegans, RNAi is gaining increasing recognition as a technique that may be used in mammalian cell. It is contemplated that RNAi may be used to disrupt the expression of a gene in a tissue-specific manner. By placing a gene fragment encoding the desired dsRNA behind an inducible or tissue-specific promoter, it should be possible to inactivate genes at a particular location within an organism or during a particular stage of development. Recently, RNAi has been used to elicit gene-specific silencing in cultured mammalian cells using 21-nucleotide siRNA duplexes (Elbashir et al., Nature, 411:494-498, 2001). In the same cultured cell systems, transfection of longer stretches of dsRNA yielded considerable nonspecific silencing. Thus, RNAi has been demonstrated to be a feasible technique for use in mammalian cells and could be used for assessing gene function in cultured cells and mammalian systems, as well as for development of gene-specific therapeutics.

Anti-sense RNA and DNA molecules, ribozymes, RNAi and triple helix molecules can be prepared by any method known in the art for the synthesis of DNA and RNA molecules. These include techniques for chemically synthesizing oligodeoxyribonucleotides well known in the art including, but not limited to, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably or transiently into cells.

C. Recombinant Protein Production.

Given the above disclosure of SHIV A proteins and SHIV A protein encoding nucleic acid constructs, it is possible to produce SHIV A protein by recombinant techniques. A variety of expression vector/host systems may be utilized to contain and express a SHIV A protein coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein production include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of SHIV A protein in bacteria, yeast and other invertebrates are described herein below.

The DNA sequence encoding the mature form of the protein is amplified by PCR and cloned into an appropriate vector for example, pGEX 3X (Pharmacia, Piscataway, N.J.). The pGEX vector is designed to produce a fusion protein comprising glutathione S transferase (GST), encoded by the vector, and a protein encoded by a DNA fragment inserted into the vector's cloning site. The primers for the PCR may be generated to include for example, an appropriate cleavage site.

Treatment of the recombinant fusion protein with thrombin or factor Xa (Pharmacia, Piscataway, N.J.) is expected to cleave the fusion protein, releasing the proapoptotic factor from the GST portion. The pGEX 3X/SHIV A protein construct is transformed into E. coli XL 1 Blue cells (Stratagene, La Jolla Calif.), and individual transformants were isolated and grown. Plasmid DNA from individual transformants is purified and partially sequenced using an automated sequencer to confirm the presence of the desired SHIV A protein-encoding gene insert in the proper orientation.

Knowledge of SHIV A protein encoding DNA sequences allows for modification of cells to permit or increase expression of endogenous SHIV A protein. The cells can be modified (heterologous promoter is inserted in such a manner that it is operably linked to, e.g., by homologous recombination) to provide increase SHIV A protein expression by replacing, in whole or in part the naturally occurring promoter with all or part of a heterologous promoter so that the cells express SHIV A protein at higher levels. The heterologous promoter is inserted in such a mariner that it is operably linked to SHIV A protein encoding sequences. (e.g., PCT International Publication No. WO96/12650; PCT International Publication No. WO 92/20808 and PCT International Publication No. WO 91/09955). It is contemplated that, in addition to the heterologous promoter DNA, amplifiable marker DNA (e.g., ada, dhfr and the multifunctional CAD gene which encodes carbamyl phosphate synthase, aspartate transcarbamylase and dihydroorotase) and/or intron DNA may be inserted along with the heterologous promoter DNA. If linked to the SHIV A protein coding sequence, amplification of the marker DNA by standard selection methods results in co amplification of the SHIV A protein coding sequences in the cells.

While certain embodiments of the present invention contemplate producing the SHIV A protein using synthetic peptide synthesizers and subsequent FPLC analysis and appropriate refolding of the cysteine double bonds, it is contemplated that recombinant protein production also may be used to produce the SHIV A protein compositions. For example, induction of the GST/SHIV A fusion protein is achieved by growing the transformed XL 1 Blue culture at 37° C. in LB medium (supplemented with carbenicillin) to an optical density at wavelength 600 nm of 0.4, followed by further incubation for 4 hours in the presence of 0.5 mM Isopropyl β-D Thiogalactopyranoside (Sigma Chemical Co., St. Louis Mo.).

The fusion protein, expected to be produced as an insoluble inclusion body in the bacteria, may be purified as follows. Cells are harvested by centrifugation; washed in 0.15 M NaCl, 10 mM Tris, pH 8, 1 mM EDTA; and treated with 0.1 mg/ml lysozyme (Sigma Chemical Co.) for 15 minutes at room temperature. The lysate is cleared by sonication and cell debris is pelleted by centrifugation for 10 minutes at 12,000×g. The fusion protein containing pellet is resuspended in 50 mM Tris, pH 8, and 10 mM EDTA, layered over 50% glycerol, and centrifuged for 30 min. at 6000×g. The pellet is resuspended in standard phosphate buffered saline solution (PBS) free of Mg++ and Ca++. The fusion protein is further purified by fractionating the resuspended pellet in a denaturing SDS polyacrylamide gel (Sambrook et al., supra). The gel is soaked in 0.4 M KCl to visualize the protein, which is excised and electroeluted in gel running buffer lacking SDS. If the GST/SHIV A protein is produced in bacteria as a soluble protein, it may be purified using the GST Purification Module (Pharmacia Biotech).

The fusion protein may be subjected to thrombin digestion to cleave the GST from the mature SHIV A protein. The digestion reaction (20-40 μg fusion protein, 20-30 units human thrombin (4000 U/mg (Sigma) in 0.5 ml PBS) is incubated 16-48 hrs at room temperature and loaded on a denaturing SDS PAGE gel to fractionate the reaction products. The gel is soaked in 0.4 M KCl to visualize the protein bands. The identity of the protein band corresponding to the expected molecular weight of SHIV A protein may be confirmed by partial amino acid sequence analysis using an automated sequencer (Applied Biosystems Model 473A, Foster City, Calif.).

Alternatively, the DNA sequence encoding the predicted mature SHIV A protein may be cloned into a plasmid containing a desired promoter and, optionally, a leader sequence (see, e.g., Better et al., Science, 240: 1041 43, 1988). The sequence of this construct may be confirmed by automated sequencing. The plasmid is then transformed into E. coli strain MC1061 using standard procedures employing CaCl2 incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the mature SHIV A protein and be cleaved during secretion.

The secreted recombinant protein is purified from the bacterial culture media by standard protein purification techniques well known to those of skill in the art.

Similarly, a yeast system may be employed to generate the recombinant peptide. This may be performed using standard commercially available expression systems, e.g., the Pichia Expression System (Invitrogen, San Diego, Calif.), following the manufacturer's instructions. This system relies on the pre pro alpha sequence to direct secretion, and transcription of the insert is driven by the alcohol oxidase (AOX1) promoter upon induction by methanol. The secreted recombinant protein is purified from the yeast growth medium by standard protein purification methods.

Alternatively, the cDNA encoding SHIV A protein may be cloned into the baculovirus expression vector pVL1393 (PharMingen, San Diego, Calif.). This vector is then used according to the manufacturer's directions (PharMingen) to infect Spodoptera frugiperda cells in sF9 protein free media and to produce recombinant protein. The protein is purified and concentrated from the media using a heparin Sepharose column (Pharmacia, Piscataway, N.J.) and sequential molecular sizing columns (Amicon, Beverly, Mass.), and resuspended in PBS. SDS PAGE analysis shows a single band and confirms the size of the protein, and Edman sequencing on a Porton 2090 Peptide Sequencer confirms its N terminal sequence.

Alternatively, the SHIV A protein may be expressed in an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The SHIV A protein coding sequence is cloned into a nonessential region of the virus, such as the polyhedrin gene, and placed under control of the polyhedrin promoter. Successful insertion of sequence will render the polyhedrin gene inactive and produce recombinant virus lacking coat protein coat. The recombinant viruses are then used to infect S. frugiperda cells or Trichoplusia larvae in which SHIV A protein is expressed (Smith et al., J Virol 46: 584, 1983; Engelhard EK et al., Proc Nat Acad Sci 91: 3224-7, 1994).

Mammalian host systems for the expression of the recombinant protein also are well known to those of skill in the art. Host cell strains may be chosen for a particular ability to process the expressed protein or produce certain post translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; als which confers resistance to chlorsulfuron; and hygro, that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, glucuronidase and its substrate, GUS, and luciferase and its substrate, luciferin.

D. Vectors for Cloning, Gene Transfer and Expression.

As discussed in the previous section, expression vectors are employed to express the SHIV A protein product, which can then be purified and, for example, be used to vaccinate animals to generate antisera or monoclonal antibody with which further studies may be conducted. In other embodiments, expression vectors may be used in gene therapy applications to introduce SHIV A protein encoding nucleic acids into cells in need thereof and/or to induce SHIV A protein expression in such cells. The present section is directed to a description of the production of such expression vectors.

Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products also are provided, as is an element that links expression of the drug selection markers to expression of the polypeptide.

a. Regulatory Elements.

Promoters and Enhancers. Throughout this application, the term “expression construct” or “expression vector” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Promoters are composed of discrete functional modules, each consisting of approximately 7-20 bp of DNA, and containing one or more recognition sites for transcriptional activator or repressor proteins.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well known properties, the level and pattern of expression of the protein of interest following transfection or transformation can be optimized.

Inducible promoter systems may be used in the present invention, e.g., inducible ecdysone system (Invitrogen, Carlsbad, Calif.), which is designed to allow regulated expression of a gene of interest in mammalian cells. Another inducible system that would be useful is the Tet-Off™ or Tet-On™ system (Clontech, Palo Alto, Calif.) originally developed by Gossen and Bujard (Gossen and Bujard, Proc Natl Acad Sci USA. 15;89(12):5547 51, 1992; Gossen et al., Science, 268(5218):1766 9, 1995).

In some circumstances, it may be desirable to regulate expression of a transgene in a gene therapy vector. For example, different viral promoters with varying strengths of activity may be utilized depending on the level of expression desired. In mammalian cells, the CMV immediate early promoter is often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate.

In certain indications, it may be desirable to activate transcription at specific times after administration of the gene therapy vector. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen (Kageyama et al., J Biol Chem. 262(5):2345 51, 1987), c-fos, TNF-alpha, C-reactive protein (Arcone et al., Nucleic Acids Res. 16(8):3195 207, 1988), haptoglobin (Oliviero et al., EMBO J. 6(7):1905 12, 1987), serum amyloid A2, C/EBP alpha, IL-1, IL-6 (Poli and Cortese, Proc Natl Acad Sci USA. 86(21):8202 6, 1989), Complement C3 (Wilson et al., Mol Cell Biol. 10(12):6181 91, 1990), IL-8, alpha-I acid glycoprotein (Prowse and Baumann, Mol Cell Biol. 8(1):42 51, 1988), alpha-1 antitypsin, lipoprotein lipase (Zechner et al., Mol Cell Biol. 8(6):2394 401, 1988), angiotensinogen (Ron et al., Mol Cell Biol. 11(5):2887 95, 1991), fibrinogen, c-jun (inducible by phorbol esters, TNF-alpha, UV radiation, retinoic acid, and hydrogen peroxide), collagenase (induced by phorbol esters and retinoic acid), metallothiohein (heavy metal and glucocorticoid inducible), Stromelysin (inducible by phorbol ester, interleukin-1 and EGF), alpha-2 macroglobulin and alpha-1 antichymotrypsin.

It is envisioned that cell cycle regulatable promoters may be useful in the present invention. For example, in a bicistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2F1, p53 and BRCA1 could be used.

Tumor specific promoters such as osteocalcin, hypoxia-responsive element (HRE), MAGE-4, CEA, alpha-fetoprotein, GRP78/BiP and tyrosinase may also be used to regulate gene expression in tumor cells. Other promoters that could be used according to the present invention include Lac-regulatable, chemotherapy inducible (e.g. MDR), and heat (hyperthermia) inducible promoters, radiation-inducible (e.g., EGR (Joki et al., Hum Gene Ther. 6(12):1507 13 1995), Alpha-inhibin, RNA pol III tRNA met and other amino acid promoters, U1 snRNA (Bartlett et al., Proc Natl Acad Sci USA. 20;93(17):8852 7, 1996), MC-1, PGK, β-actin and γ-globin. Many other promoters that may be useful are listed in Walther and Stein (J Mol Med. 74(7):379 92, 1996).

It is envisioned that any of the above promoters alone or in combination with another may be useful according to the present invention depending on the action desired. In addition, this list of promoters should not be construed to be exhaustive or limiting, and those of skill in the art will know of other promoters that may be used in conjunction with the promoters and methods disclosed herein.

Another regulatory element contemplated for use in the present invention is an enhancer. These are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization. Enhancers useful in the present invention are well known to those of skill in the art and will depend on the particular expression system being employed (Scharf D et al Results Probl Cell Differ 20: 125-62, 1994; Bittner et al Methods in Enzymol 153: 516-544, 1987).

Polyadenylation Signals. Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

IRES. In certain embodiments of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites (Pelletier and Sonenberg, Nature, 334:320-325, 1988). IRES elements from two members of the picornavinis family (poliovirus and encephalomyocarditis) have been described (Pelletier and Sonenberg, 1988 supra), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers.

In this way, expression of several proteins can be simultaneously engineered into a cell with a single construct and a single selectable marker.

b. Delivery of Expression Vectors.

There are a number of ways in which expression constructs may introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genome and express viral genes stably and efficiently have made them attractive candidates for the transfer of foreign genes into mammalian cells (Ridgeway, In: Rodriguez R L, Denhardt D T, ed. Vectors: A survey of molecular cloning vectors and their uses. Stoneham: Butterworth, 467 492, 1988; Nicolas and Rubenstein, In: Vectors: A survey of molecular cloning vectors and their uses, Rodriguez & Denhardt (eds.), Stoneham: Butterworth, 493 513, 1988; Baichwal and Sugden, In: Gene Transfer, Kucherlapati R, ed., New York, Plenum Press, 117 148, 1986; Temin, In: gene Transfer, Kucherlapati (ed.), New York: Plenum Press, 149 188, 1986). The first viruses used as gene vectors were DNA viruses including the papovaviruses (simian virus 40, bovine papilloma virus, and polyoma)(Ridgeway, 1988 supra; Baichwal and 0.20. Sugden, 1986 supra) and adenoviruses (Ridgeway, 1988 supra; Baichwal and Sugden, 1986 supra). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988 supra; Temin, 1986 supra).

It is now widely recognized that DNA may be introduced into a cell using a variety of viral vectors. In such embodiments, expression constructs comprising viral vectors containing the genes of interest may be adenoviral (see for example, U.S. Pat. Nos. 5,824,544; 5,707,618; 5,693,509; 5,670,488; 5,585,362; each incorporated herein by reference), retroviral (see for example, U.S. Pat. Nos. 5,888,502; 5,830,725; 5,770,414; 5,686,278; 4,861,719 each incorporated herein by reference), adeno-associated viral (see for example, U.S. Pat. Nos. 5,474,935; 5,139,941; 5,622,856; 5,658,776; 5,773,289; 5,789,390; 5,834,441; 5,863,541; 5,851,521; 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. Nos. 5,879,934; 5,849,571; 5,830,727; 5,661,033; 5,328,688 each incorporated herein by reference) vector.

There are a number of alternatives to viral transfer of genetic constructs. This section provides a discussion of methods and compositions of non-viral gene transfer. DNA constructs of the present invention are generally delivered to a cell, and in certain situations, the nucleic acid or the protein to be transferred may be transferred using non-viral methods.

Several non-viral methods for the transfer of expression constructs into cultured mammalian cells are contemplated by the present invention. These include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA; 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraleyetal., Proc. Natl. Acad. Sci. USA, 76:3348-3352, 1979; Felgner, Sci Am. 276(6):102 6, 1997; Felgner, Hum Gene Ther. 7(15):1791 3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

Once the construct has been delivered into the cell, the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. The addition of DNA to cationic liposomes causes a topological transition from liposomes to optically birefringent liquid-crystalline condensed globules (Radler et al., Science, 275(5301):810 4, 1997). These DNA-lipid complexes are potential non-viral vectors for use in gene therapy and delivery. Liposome-mediated nucleic acid delivery and expression of foreign DNA in vitro has been very successful. Also contemplated in the present invention are various commercial approaches involving “lipofection” technology. Complexing the liposome with a hemagglutinating virus (HVJ) may facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987, supra) and transferrin (Wagner et al., Proc. Nat'l. Acad. Sci. USA, 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Natl. Acad. Sci., USA 91:4086-4090, 1994) and epidermal growth factor (EGF) has also been used to deliver genes to squamous carcinoma cells (Myers, EPO 0273085).

In other embodiments, the delivery vehicle may comprise a ligand and a liposome. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (Proc. Nat. Acad. Sci. USA, 81:7529-7533, 1984; Benvenisty and Neshif(Proc. Nat. Acad. Sci. USA, 83:9551-9555, 1986).

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

E. Antibodies Immunoreactive with SHIV A Protein.

In another aspect, the present invention contemplates an antibody that is immunoreactive with a SHIV A protein molecule of the present invention, or any portion thereof. Such antibodies include, but are not limited to, polyclonal, monoclonal, chimeric, single chain, Fab fragments and fragments produced by a Fab expression library, bifunctional/bispecific antibodies, humanized antibodies, CDR grafted antibodies, human antibodies and antibodies which include portions of CDR sequences specific for SHIV A protein.

Neutralizing antibodies, i.e., those which inhibit apoptotic activity of SHIV A, are especially preferred for therapeutic embodiments. In a preferred embodiment, an antibody is a monoclonal antibody. The invention provides for a pharmaceutical composition comprising a therapeutically effective amount of an antibody directed against SHIV A protein. The antibody may bind to and neutralize the apoptotic effects of the SHIV A protein. The antibody may be formulated with a pharmaceutically acceptable adjuvant. Means for preparing and characterizing antibodies are well known in the art (see, e.g., Harlow and Lane, ANTIBODIES: A LABORATORY MANUAL, Cold Spring Harbor Laboratory, Cold Spring Harbor, N.Y., 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, goat, sheep, pigs or horses. Because of the relatively large blood volume of rabbits, a rabbit is a preferred choice for production of polyclonal antibodies.

Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Antibodies, both polyclonal and monoclonal, specific for isoforms of antigen may be prepared using conventional immunization techniques, as will be generally known to those of skill in the art. As used herein, the term “specific for” is intended to mean that the variable regions of the antibodies recognize and bind SHIV A protein and are capable of distinguishing SHIV A protein from other antigens, for example other secreted proapoptotic factors. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

Monoclonal antibodies to SHIV A protein may be prepared using any technique which provides for the production of antibody molecules by continuous cell lines in culture. These include but are not limited to the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R L,iss Inc, New York N.Y., pp 77-96, (1985).

When the hybridoma technique is employed, myeloma cell lines may be used. Such cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, F0, NSO/U, MPC-11, MPC₁₋₁-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions. It should be noted that the hybridomas and cell lines produced by such techniques for producing the monoclonal antibodies are contemplated to be novel compositions of the present invention. An exemplary method for producing monoclonal antibodies against SHIV A is provided in Example 1. Those of skill in the art will appreciate that such a method may be modified using techniques well known to those of skill in the art and still produce antibodies within the scope of the present invention.

In addition to the production of monoclonal antibodies, techniques developed for the production of “chimeric antibodies”, the splicing of mouse antibody genes to human antibody genes to obtain a molecule with appropriate antigen specificity and biological activity can be used (Morrison et al., Proc Natl Acad Sci 81: 6851-6855, 1984; Neuberger et al. Nature 312: 604-608, 1984; Takeda et al., Nature 314: 452-454; 1985). Alternatively, techniques described for the production of single chain antibodies (U.S. Pat. No. 4,946,778) can be adapted to produce SHIV A protein-specific single chain antibodies.

Antibodies may also be produced by inducing in vivo production in the lymphocyte population or by screening recombinant immunoglobulin libraries or panels of highly specific binding-reagents as disclosed in Orlandi et al (Proc Natl Acad Sci 86: 3833-3837; 1989), and Winter G and Milstein C (Nature 349: 293-299, 1991).

It is proposed that the antibodies of the present invention will find useful application in standard immunochemical procedures, such as ELISA and Western blot methods and in immunohistochemical procedures such as tissue staining, as well as in other procedures which may utilize antibodies specific to SHIV A protein-related antigen epitopes. Additionally, it is proposed that monoclonal antibodies specific to the particular SHIV A protein of different species may be utilized in other useful applications.

In general, both polyclonal and monoclonal antibodies against SHIV A protein may be used in a variety of embodiments. In certain aspects, the antibodies may be employed for therapeutic purposes in which the inhibition of SHIV A protein activity is desired (e.g., to reduce apoptosis in neuronal cells). Antibodies may be used to block SHIV A protein action. In doing so, these antibodies can be used to ameliorate SHIV A-mediated apoptosis, thereby reducing tissue destruction.

Antibodies of the present invention also may prove useful in diagnostic purposes in order, for example, to detect increases or decreases in SHIV A protein in tissue samples including samples for sites of inflammation, or fluid samples including blood serum, plasma and exudate samples. Additional aspects will employ the antibodies of the present invention in antibody cloning protocols to obtain cDNAs or genes encoding other SHIV A protein. They may also be used in inhibition studies to analyze the effects of SHIV A related peptides in cells or animals. Anti-SHIV A protein antibodies will also be useful in immunolocalization studies to analyze the distribution of SHIV A protein during various cellular events, for example, to determine the cellular or tissue-specific distribution of SHIV A protein polypeptides under different points in the cell cycle. A particularly useful application of such antibodies is in purifying native or recombinant SHIV A protein, for example, using an antibody affinity column. The operation of all such immunological techniques will be known to those of skill in the art in light of the present disclosure.

F. Use of SHIV A-based Compositions for Diagnostic Purposes.

Preferred aspects of the present invention are directed to methods of diagnosing HIV-1 infection in an individual. Such diagnostic methods may yield useful information even in the absence of detectable viral load.

In preferred embodiments, the diagnostic methods of the present invention are achieved through the detection of the 6 kDa fragment produced by SHIV A. Such a protein may be detected using antibodies specific for the protein in any of a number of formats commonly used by those of skill in the art for such detection.

For example, elsewhere in the present application, the production and characterization of monoclonal antibodies specific for SHIV A is described. Such antibodies may be employed in ELISA-based techniques and/Western blotting techniques to detect the presence of the full length SHIV A or the 6 kDa proapoptotic fragment thereof in a biological sample from a subject being tested. Methods for setting up ELISA assays and preparing Western blots of a sample are well known to those of skill in the art. The biological sample can be any tissue or fluid in which SHIV A cells might be present.

An anti-SHIV A antibody or fragment thereof can be used to monitor expression of this protein in HIV infected individuals, similar to the way anti-CD4 has been used as a diagnostic indicator of disease stage. Typically, diagnostic assays entail detecting the formation of a complex resulting from the binding of an antibody or fragment thereof to SHIV A. For diagnostic purposes, the antibodies or antigen-binding fragments can be labeled or unlabeled. The antibodies or fragments can be directly labeled. A variety of labels can be employed, including, but not limited to, radionuclides, fluorescers, enzymes, enzyme substrates, enzyme cofactors, enzyme inhibitors and ligands (e.g., biotin, haptens). Numerous appropriate immunoassays are known to the skilled artisan (see, for example, U.S. Pat. Nos. 3,817,827; 3,850,752; 3,901,654 and 4,098,876). When unlabeled, the antibodies or fragments can be detected using suitable means, as in agglutination assays, for example. Unlabeled antibodies or fragments can also be used in combination with another (i.e., one or more) suitable reagent which can be used to detect antibody, such as a labeled antibody (e.g., a second antibody) reactive with the first antibody (e.g., anti-idiotype antibodies or other antibodies that are specific for the unlabeled immunoglobulin) or other suitable reagent (e.g., labeled protein A).

In one embodiment, the antibodies or fragments of the present invention can be utilized in enzyme immunoassays, wherein the subject antibody or fragment, or second antibodies, are conjugated to an enzyme. When a biological sample comprising a SHIV A protein is combined with the subject antibodies, binding occurs between the antibodies and the SHIV A protein. In one embodiment, a biological sample containing cells expressing a mammalian SHIV A protein, or biological fluid containing secreted SHIV A is combined with the subject antibodies, and binding occurs between the antibodies and the SHIV A protein present in the biological sample comprising an epitope recognized by the antibody. These bound protein can be separated from unbound reagents and the presence of the antibody-enzyme conjugate specifically bound to the SHIV A protein can be determined, for example, by contacting the sample with a substrate of the enzyme which produces a color or other detectable change when acted on by the enzyme. In another embodiment, the subject antibodies can be unlabeled, and a second, labeled antibody can be added which recognizes the subject antibody.

Kits for use in detecting the presence of a mammalian SHIV A protein in a biological sample can also be prepared. Such kits will include an antibody or functional fragment thereof which binds to a mammalian SHIV A protein or portion of this protein, as well as one or more ancillary reagents suitable for detecting the presence of a complex between the antibody or fragment and SHIV A or portion thereof. The antibody compositions of the present invention can be provided in lyophilized form, either alone or in combination with additional antibodies specific for other epitopes. The antibodies, which can be labeled or unlabeled, can be included in the kits with adjunct ingredients (e.g., buffers, such as Tris, phosphate and carbonate, stabilizers, excipients, biocides and/or inert proteins, e.g., bovine serum albumin). For example, the antibodies can be provided as a lyophilized mixture with the adjunct ingredients, or the adjunct ingredients can be separately provided for combination by the user. Generally these adjunct materials will be present in less than about 5% weight based on the amount of active antibody, and usually will be present in a total amount of at least about 0.001% weight based on antibody concentration. Where a second antibody capable of binding to the monoclonal antibody is employed, such antibody can be provided in the kit, for instance in a separate vial or container. The second antibody, if present, is typically labeled, and can be formulated in an analogous manner with the antibody formulations described above.

Similarly, the present invention also relates to a method of detecting and/or quantitating expression of a mammalian SHIV A protein or a portion of the SHIV A protein by a cell, in which a composition comprising a cell or fraction thereof (e.g., a soluble fraction) is contacted with an antibody or functional fragment thereof which binds to a mammalian SHIV A protein or a portion of the SHIV A protein (e.g., a 6 kD protein derived from SHIV A or a protein of SEQ ID NO:3) under conditions appropriate for binding of the antibody or fragment thereto, and binding is monitored. Detection of the antibody, indicative of the formation of a complex between antibody and or a portion of the protein, indicates the presence of the protein.

The method can be used to detect expression of SHIV A from the cells of an individual (e.g., in a sample, such as a body fluid, such as blood, saliva or other suitable sample). The level of expression of in a biological sample of that individual can also be determined, for instance, by flow cytometry, and the level of expression (e.g., staining intensity) can be correlated with disease susceptibility, progression or risk.

In other embodiments, the present invention also contemplates functional assays for determining the presence of SHIV A in a given biological sample. In such embodiments, the biological sample obtained from the individual being tested may be incubated with a T cell population or a B cell population. Monitoring the effects of the biological sample on these cell populations should reveal whether the biological sample contains an apoptotic factor. If the cells undergo apoptosis, the biological sample is positively identified as containing such a factor.

In certain other diagnostic embodiments, the polynucleotide sequences encoding SHIV A protein may be used for the diagnosis of conditions or diseases with which the expression of SHIV A protein is associated. One aspect of the present invention comprises a method for identifying method of diagnosing HIV infection in a subject comprising obtaining a biological sample from the subject and determining the increased expression of a SHIV A protein in the biological sample by amplifying and detecting nucleic acids corresponding to nucleic acids that encode SHIV A protein. The biological sample can be any tissue or fluid in which SHIV A cells might be present. Preferred embodiments include macrophages, neuronal cells, central nervous system cells, microglial cells, glial cells, T-cells, and B-cells. Other embodiments include samples where the body fluid is blood, lymph fluid, ascites, serous fluid, pleural effusion, sputum, cerebrospinal fluid, lacrimal fluid, or urine.

In the amplification procedures, polynucleotide sequences encoding SHIV A protein may be used in hybridization or PCR assays of fluids or tissues from biopsies to detect SHIV A protein expression. Such methods may be qualitative or quantitative in nature and may include Southern or northern analysis, dot blot or other membrane-based technologies; PCR technologies, dip stick, pin, chip and ELISA technologies. All of these techniques are well known in the art and are the basis of many commercially available diagnostic kits.

In addition such assays may useful in evaluating the efficacy of a particular therapeutic treatment regime in animal studies, in clinical trials, or in monitoring the treatment of an individual patient. In order to provide a basis for the diagnosis of disease, a normal or standard profile for SHIV A protein expression needs to be established. This generally involves a combination of body fluids or cell extracts taken from normal subjects, either animal or human, with SHIV A protein, or a portion thereof, under conditions suitable for hybridization or amplification. Standard hybridization may be quantified by comparing the values obtained for normal subjects with a dilution series of SHIV A protein run in the same experiment where a known amount of purified SHIV A protein is used. Standard values obtained from normal samples may be compared with values obtained from samples from cachectic subjects affected by SHIV A protein expression. Deviation between standard and subject values establishes the presence of disease.

Once disease is established, a therapeutic agent is administered; and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

PCR as described in U.S. Pat. Nos. 4,683,195 and 4,965,188 provides additional uses for oligonucleotides based upon the SHIV A protein sequence. Such oligomers are generally chemically synthesized, but they may be generated enzymatically or produced from a recombinant source as described herein above. Oligomers generally comprise two nucleotide sequences, one with sense orientation and one with antisense, employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Other nucleic acid amplification procedures include transcription-based amplification systems (TAS), including nucleic acid sequence based amplification (NASBA) and 3SR. Kwoh et al., 1989; Gingeras et al., PCT Application WO 88/10315, incorporated herein by reference in their entirety. Another method for amplification is the ligase chain reaction (“LCR”), disclosed in EPO No. 320 308, incorporated herein by reference in its entirety. U.S. Pat. No. 4,883,750 describes a method similar to LCR for binding probe pairs to a sequence. Strand Displacement Amplification (SDA) is another method of carrying out isothermal amplification of nucleic acids which involves multiple rounds of strand displacement and synthesis, i.e., nick translation. A similar method, called Repair Chain Reaction (RCR) involves annealing several probes throughout a region targeted for amplification, followed by a repair reaction in which only two of the four bases are present. The other two bases can be added as biotinylated derivatives for easy detection. A similar approach is used in SDA. Specific sequences can also be detected using a cyclic probe reaction (CPR). In CPR, a probe having a 3′ and 5′ sequences of nonspecific DNA and middle sequence of specific RNA is hybridized to DNA which is present in a sample. Upon hybridization, the reaction is treated with RNaseH, and the products of the probe identified as distinctive products which are released after digestion. The original template is annealed to another cycling probe and the reaction is repeated.

Following amplification, it may be desirable to separate the amplification product from the template and the excess primer for the purpose of determining whether specific amplification occurred. In a preferred embodiment, amplification products are separated by agarose, agarose-acrylamide or polyacrylamide gel electrophoresis using standard methods. See Sambrook et al., 1989. In a preferred embodiment, the gel is a 2% agarose gel.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography.

The amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

However, it also is possible to determine the sequence of the amplification products without separation. These methods may be collectively termed Sequencing By Hybridization or SBH (Cantor et al., 1992; Drmanac & Crkvenjakov, U.S. Pat. No. 5,202,231). Development of certain of these methods has given rise to new solid support type sequencing tools known as sequencing chips. These techniques are described in numerous U.S. Patents including e.g., U.S. Pat. Nos. 5,202,231; 6,401,267 and also WO 89/10977.

In certain embodiments, the amplification products are visualized indirectly. Following separation of amplification products, a nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety.

In a particularly preferred embodiment, detection is by Southern blotting and hybridization with a labeled probe, according to standard protocol. See Sambrook et al., 1989. In such methods, the amplification products are separated by gel electrophoresis. The gel is then contacted with a membrane, such as nitrocellulose, permitting transfer of the nucleic acid and non-covalent binding. Subsequently, the membrane is incubated with a chromophore conjugated probe that is capable of hybridizing with a target amplification product. Detection is by exposure of the membrane to x-ray film or ion-emitting detection devices. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out the diagnostic (and prognostic) methods according to the present invention.

Additionally, methods to quantitate the expression of a particular molecule include radiolabeling (Melby et al., J Immunol Methods 159: 235-44, 1993) or biotinylating (Duplaa et al., Anal Biochem 229-36, 1993) nucleotides, coamplification of a control nucleic acid, and standard curves onto which the experimental results are interpolated. Quantitation of multiple samples may be speeded up by running the assay in an ELISA format where the oligomer of interest is presented in various dilutions and a spectrophotometric or calorimetric response gives rapid quantitation. For example, the presence of SHIV A protein in extracts of biopsied tissues may indicate the onset of a particular disease. A definitive diagnosis of this type may allow health professionals to begin aggressive treatment and prevent further worsening of the condition.

In addition to being used as diagnostic methods, the above-articulated methods also may be used in a prognostic manner to monitor the efficacy of treatment of HAD or other disorder in which the expression of SHIV A is being modulated. The methods may be performed immediately before, during and after treatment to monitor treatment success. The methods also should be performed at intervals, preferably every three to six months, on disease free patients to insure treatment success.

G. Functional Assays to Monitor Apoptosis.

In certain aspects of the present invention it may be necessary to determine the apoptotic activity of SHIV A protein and any variants thereof. There are numerous assays for determining apoptotic activity that are well known to those of skill in the art, however, merely by way of example, certain such assays are described in this section.

Apoptosis is a form of cell death (programmed cell death) that exhibits stereotypic morphological changes as reviewed in Raff, Nature, 396:119-122, 1998. Apoptotic cell death is characterized by cellular shrinkage, chromatin condensation, cytoplasmic blebbing, increased membrane permeability and interchromosomal DNA cleavage. Kerr et al., FASEB J. 6:2450, 1992; Cohen and Duke, Ann. Rev. Immunol. 10:267, 1992. The blebs, small, membrane-encapsulated spheres that pinch off of the surface of apoptotic cells, may continue to produce superoxide radicals which damage surrounding cell tissue and may be involved in inflammatory processes. Apoptosis is distinct from necrotic cell death which results in cell swelling and release of intracellular components (Kerr et al., Br. J. Cancer, 26, 239-257 (1972); Wyllie et al., Int. Rev. Cytol., 68, 251-306 (1980); Wyllie, Nature, 284, 555-556, 1980). Apoptotic cells, without releasing such components, are phagocytosed and hence degraded (Savill et al., Nature, 343, 170-173, 1990).

Cellular apoptotic responses can be monitored in a number of ways, including analysis of chromosomal DNA fragmentation, fluorescence-activated cell sorting (FACS) of propidium iodide-stained cells (Dengler et al., Anticancer Drugs, 6:522-32, 1995), in situ terminal deoxynucleotidyl transferase and nick translation assay (TUNEL analysis) described in Gorczyca, Cancer Res. 53:1945-51, 1993 and measurement of caspase activation. For example, an apoptotic response can be determined by staining the chromosomal DNA of treated cells with propidium iodide and analyzing the individual cells by FACS (FACS-Calibur, Becton-Dickinson; Mountain View, Calif.). Typical cell culture populations display a large peak of cells in the G1/G0 phase of the cell cycle, with a smaller peak representing G2/M phase cells. Between these 2 peaks are cells in the S phase of the cell cycle. Cells which exhibit DNA labeling which is before the G 1/G0 peak represent cells with fragmented DNA comprising less than the diploid amount of chromosomal DNA, and thus, undergoing cell death (Dengler, et al., 1995). This measurement gives a relative quantification of apoptosis that is comparable to other apoptosis assays including TdT-mediated dUTP nick-end labeling (TUNEL analysis); Gorczyca et al., (1993) Cancer Res. 53: 1945-51.

Tunel analysis for cell apoptosis is well known to those of skill in the art, and kits for performing this assay are commercially available. For example, the “In situ cell death detection kit AP” is available from Boehringer Mannheim, and ApopTag in situ apoptosis detection kit is available from Oncor, Gaithersburg, Md.). In such an assay, frozen tissue sections are fixed by 4% paraformaldehyde (available from Sigma Chemicals, St. Louis, Mo.), washed three times with PBS (phosphate buffered saline) and brought into contact with a TUNEL (TdT using nick end labeling) reaction mixture to label a DNA chain degradation product. The mixture is then allowed to stand at 37° C. for an appropriate amount of time e.g. 45 minutes, and then washed with PBS, to which converter-AP is added and the mixture is treated at 37° C. for 60 minutes. In conventional manners, dehydration, clearing and mounting are conducted to observe apoptosis using light microscopy. Chromatin of apoptosis cells appears condensed in the form of meniscus around nuclear membrane, and purple-blue apoptotic body is observed. Apoptosis positive cells can be observed and the number of the cells per one mm length can be counted under a light microscope at e.g., magnification of 400.

DNA fragmentation can be monitored by gel electrophoresis. Samples may be analysed by gel electrophoresis on e.g. a 1-2% agarose gel, using an appropriate DNA ladder as a molecular size standard (Sigma Chemicals, St. Louis. Mo.). 100 V are applied for 1 hour, and then the gel was stained in ethidium bromide for 20 min to enable visualization of the internucleosomal DNA fragments.

Apoptosis also is commonly measured using assays for caspase activation. Caspases are an evolutionarily conserved family of enzymes which proteolytically degrade and dissemble the cell in response to proapoptotic signals (reviewed in Thornberry and Lazebnik, 1998). Apoptosis can be evaluated using this distinct biochemical end-point, by measuring caspase activity in cell lysates prepared from the cell of interest using a fluorometric assay for caspase 3 activity (Apo-Alert CPP32/Caspase-3 Assay; Clontech). An increased caspase activity above the background level of caspase activity in untreated cells is indicative of a proapoptotic effect.

The Apo-ONE™ Homogeneous Caspase-3/7 Assay (Cat.# G7790; Promega, Madison, Wis.) is another commercially available system for the measurement of both caspase-3 and caspase-7 activities. The kit is comprised of two components: the Caspase Substrate Z-DEVD-R110 and the Apo-ONE™ Homogeneous Caspase-3/7 Buffer. These two components are mixed into a single homogeneous caspase reagent that can be added directly to samples being tested.

After initiating apoptosis, most cell types translocate phosphatidylserine (PS) from the inner face of the plasma membrane to the cell surface (Martin, et al., J. Exp. Med.182: 1545-1556, 1995). The ApoAlert Annexin V assay, commercially available from Clontech Laboratories, Inc., exploits this observation. Once on the cell surface, PS can be easily detected by staining with a FITC conjugate of annexin V, a protein that has a strong natural affinity for PS. Externalization of PS occurs earlier than the nuclear changes associated with apoptosis, so the ApoAlert Assay may be used to detect apoptotic cells. The cells are stained with annexin VFITC and the apoptotic cells are clearly visible by fluorescence microscopy after exposure to staurospbrine for one hour. Screening by microscopy may be used to test putative apoptosis inducers or inhibitors, or to look at the time course of apoptosis.

H. Screening for Modulators of SHIV A Protein.

The present invention also contemplates the use of SHIV A protein and active fragments thereof in the screening of compounds that modulate (increase or decrease activity) of SHIV A protein. These assays may make use of a variety of different formats and may depend on the kind of “activity” for which the screen is being conducted. Contemplated functional “read-outs” include SHIV A protein binding to a substrate; SHIV A protein binding to a receptor, caspase assays or any other functional assay normally employed to monitor apoptosis induced by SHIV A protein. Functional assays that determine apoptosis are well known to those of skill in the art and some exemplary assays have been described elsewhere in this document.

a. Assay Formats.

The present invention provides methods of screening for modulators of SHIV A protein activity by monitoring SHIV A-induced apoptotic activity in the presence and absence of the candidate substance and comparing such results. It is contemplated that this screening technique will prove useful in the general identification of a compound that will serve the purpose of altering the effects of SHIV A. In certain embodiments, it will be desirable to identify inhibitors of SHIV A activity. In other embodiments, it will be desirable to identify stimulators of SHIV A activity.

As discussed herein throughout, SHIV A protein induces apoptosis in neuronal cells, as well as T cells and B cells, of HIV-1 infected individuals. For example, this induction of apoptosis is exemplified in PBMC, CD⁻⁴⁺ T cells, CD-⁻⁸⁺ T cells and B cells. Inhibitors of SHIV A activity identified herein will be useful in inhibiting, decreasing or otherwise abrogating the effects of SHIV A protein. Such compounds will be useful in the treatment of HAD.

In alternative embodiments, stimulators of SHIV A will be identified that may be used for promoting, augmenting or increasing the therapeutic effects of SHIV A protein. Such compounds will be useful in the treatment of various disorders in which it is desirable to promote apoptosis. In particular, such agents that increase the activity of the protein will be useful where it is necessary to achieve B-cell depletion, such as microbial infections; allergic or asthmatic responses; mechanical injury associated with trauma; arteriosclerosis; autoimmune diseases; and leukemia, lymphomas or carcinomas. For example, U.S. Pat. No. 6,224,866 provides methods and compositions for depeleting B-cells in order to achieve a therapeutic effect. The compositions of the present invention could be employed in similar methods to achieve a therapeutic depletion of B-cells.

In the screening embodiments, the present invention is directed to a method for determining the ability of a candidate substance to alter the SHIV A protein expression or activity of cells that either naturally express SHIV A protein or have been engineered to express SHIV A protein as described herein. The method includes generally the steps of:

a. providing a cell that expresses SHIV A;

b. contacting the cell with a candidate modulator; and

c. monitoring a change in the expression or activity of SHIV A that occurs in the presence of said modulator.

In such an assay, an alteration in SHIV A protein activity, expression or processing in the presence of the candidate substance will indicate that the candidate substance is a modulator of the activity.

To identify a candidate substance as being capable of stimulating SHIV A protein activity in the assay above, one would measure or determine the activity in the absence of the added candidate substance. One would then add the candidate substance to the cell and determine the activity in the presence of the candidate substance. A candidate substance which increases the activity (e.g., apoptosis in surrounding cells, release of 6 kDa fragment, or even an increased activity that is brought about as a result of increased expression) relative to that observed in its absence is indicative of a candidate substance with stimulatory capability. It should be noted that SHIV A is produced by macrophages that are infected with HIV-1 and released therefrom. The apoptoctic effect of the SHIV A is exerted on neuronal cells, T-cells and B-cells, i.e., the cells undergoing apoptosis are not necessarily the same as the cells that are expressing the SHIV A protein.

While the above method generally describes a SHIV A protein activity, it should be understood that candidate substance may be an agent that alters the production of SHIV A protein, thereby increasing or decreasing the amount of SHIV A protein present as opposed to the per unit activity of the SHIV A protein.

Inhibitors of SHIV A protein activity or production may identified in assays set up in much the same manner as those described above in assays for SHIV A protein stimulators. In these embodiments, the present invention is directed to a method for determining the ability of a candidate substance to have an inhibitory or even antagonistic effect on SHIV A protein activity. To identify a candidate substance as being capable of inhibiting SHIV A protein activity, one would measure or determine SHIV A protein activity in the absence of the added candidate substance. One would then add the candidate inhibitory substance to the cell and determine the SHIV A protein in the presence of the candidate inhibitory substance. A candidate substance which is inhibitory would decrease the SHIV A protein activity, relative to the SHIV A protein activity in its absence.

b. Candidate Substances.

As used herein the term “candidate substance” refers to any molecule that is capable of modulating apoptosis. In specific embodiments, the molecule is one which modulates SHIV A protein activity. The candidate substance may be a protein or fragment thereof, a small molecule inhibitor, or even a nucleic acid molecule. It may prove to be the case that the most useful pharmacological compounds for identification through application of the screening assay will be compounds that are structurally related to other known modulators of apoptosis. The active compounds may include fragments or parts of naturally-occurring compounds or may be only found as active combinations of known compounds which are otherwise inactive. However, prior to testing of such compounds in humans or animal models, it will be necessary to test a variety of candidates to determine which have potential.

Accordingly, the active compounds may include fragments or parts of naturally-occurring compounds or may be found as active combinations of known compounds which are otherwise inactive. Accordingly, the present invention provides screening assays to identify agents which stimulate or inhibit cellular apoptosis. It is proposed that compounds isolated from natural sources, such as animals, bacteria, fungi, plant sources, including leaves and bark, and marine samples may be assayed as candidates for the presence of potentially useful pharmaceutical agents.

It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other inorganic or organic chemical compounds that may be designed through rational drug design starting from known stimulators or inhibitors of apoptosis.

The candidate screening assays are simple to set up and perform. Thus, in assaying for a candidate substance, after obtaining a cell expressing functional SHIV A protein, one will admix a candidate substance with the cell, under conditions which would allow measurable SHIV A protein activity to occur. In this fashion, one can measure the ability of the candidate substance to stimulate the activity of the cell in the absence of the candidate substance. Likewise, in assays for inhibitors after obtaining a cell expressing functional SHIV A protein, the candidate substance is admixed with the cell. In this fashion the ability of the candidate inhibitory substance to reduce, abolish, or otherwise diminish a biological effect mediated by SHIV A protein from said cell may be detected.

“Effective amounts” in certain circumstances are those amounts effective to reproducibly alter a given SHIV A protein mediated event e.g., apoptosis, from the cell in comparison to the normal levels of such an event. Compounds that achieve significant appropriate changes in such activity will be used.

Significant changes in SHIV A protein activity or function, e.g., in any of the apoptosis assays described herein, are represented by an increase/decrease in apoptotic activity of at least about 30%-40%, and most preferably, by changes of at least about 50%, with higher values of course being possible. The active compounds of the present invention also may be used for the generation of antibodies which may then be used in analytical and preparatory techniques for detecting and quantifying further such inhibitors.

SHIV A protein polypeptides of the invention are amendable to numerous high throughput screening (HTS) assays known in the art, including melanophore assays to investigate receptor ligand interactions, yeast based assay systems and mammalian cell expression systems. For a review see Jayawickreme and Kost, Curr. Opin. Biotechnol. 8: 629 634 (1997). Automated and miniaturized HTS assays are also contemplated as described for example in Houston and Banks Curr. Opin. Biotechnol. 8: 734 740 (1997)

There are a number of different libraries used for the identification of small molecule modulators including chemical libraries, natural product libraries and combinatorial libraries comprised or random or designed peptides, oligonucleotides or organic molecules. Chemical libraries consist of structural analogs of known compounds or compounds that are identified as hits or leads via natural product screening or from screening against a potential therapeutic target. Natural product libraries are collections of products from microorganisms, animals, plants, insects or marine organisms which are used to create mixtures of screening by, e.g., fermentation and extractions of broths from soil, plant or marine organisms. Natural product libraries include polypeptides, non-ribosomal peptides and non-naturally occurring variants thereof. For a review see Science 282:63 68 (1998). Combinatorial libraries are composed of large numbers of peptides oligonucleotides or organic compounds as a mixture. They are relatively simple to prepare by traditional automated synthesis methods, PCR cloning or other synthetic methods. Of particular interest will be libraries that include peptide, protein, peptidomimetic, multiparallel synthetic collection, recombinatorial and polypeptide libraries. A review of combinatorial libraries and libraries created therefrom, see Myers Curr. Opin. Biotechnol. 8: 701 707 (1997). A candidate modulator identified by the use of various libraries described may then be optimized to modulate activity of SHIV A protein through, for example, rational drug design.

It will, of course, be understood that all the screening methods of the present invention are useful in themselves notwithstanding the fact that effective candidates may not be found. The invention provides methods for screening for such candidates, not solely methods of finding them.

c. In Vitro Assays.

In one particular embodiment, the invention encompasses various binding assays. These can include screening for inhibitors of SHIV A protein receptor complexes or for molecules capable of binding to SHIV A protein, as a substitute of the receptor function and thereby altering the binding of the SHIV A protein to its receptor and affecting its activity. In such assays, SHIV A protein or a fragment thereof may be either free in solution, fixed to a support, expressed in or on the surface of a cell. Either the polypeptide or the binding agent may be labeled, thereby permitting determination of binding.

Such assays are highly amenable to automation and high throughput. High throughput screening of compounds is described in WO 84/03564. Large numbers of small peptide test compounds are synthesized on a solid substrate, such as plastic pins or some other surface. The peptide test compounds are reacted with SHIV A protein and washed. Bound polypeptide is detected by various methods. Combinatorial methods for generating suitable peptide test compounds are specifically contemplated.

Of particular interest in this format will be the screening of a variety of different SHIV A protein mutants. These mutants, including deletion, truncation, insertion and substitution mutants, will help identify which domains are involved with the SHIV A protein/receptor interaction. Once this region has been determined, it will be possible to identify which of these mutants, which have altered structure but retain some or all of the biological functions of SHIV A protein.

Purified SHIV A protein or a binding agent can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the SHIV A protein active region to a solid phase.

Other forms of in vitro assays include those in which functional readouts are taken. For example cells in which a wild-type or mutant SHIV A protein polypeptide is expressed can be treated with a candidate substance. In such assays, the substance would be formulated appropriately, given its biochemical nature, and contacted with the cell. Depending on the assay, culture may be required. The cell may then be examined by virtue of a number of different physiologic assays, as discussed above. Alternatively, molecular analysis may be performed in which the cells characteristics are examined. This may involve assays such as those for protein expression, enzyme function, substrate utilization, mRNA expression (including differential display of whole cell or poly A RNA) and others.

d. In Vivo Assays.

The present invention also encompasses the use of various animal models. Given the disclosure of the present invention, it will be possible to identify non-human counterparts of SHIV A protein. This will afford an excellent opportunity to examine the function of SHIV A protein in a whole animal system where it is normally expressed. By developing or identifying mice with aberrant SHIV A protein functions (overexpression of SHIV A protein, constitutively activated SHIV A protein SHIV A protein knockout animals), one can provide models that will be highly predictive of disease in humans and other mammals, and helpful in identifying potential therapies.

Another form of in vivo model is an animal with a SHIV A protein mediated disorder, e.g., as described herein below, transgenic models exhibiting HAD may be generated using the teachings of the present invention, alternatively, other models of HIV-induced secondary disorders also may prove useful. In this model, the animal is treated with SHIV A protein in combination with other agents to determine the effect on SHIV A protein function in vivo. Similarly, in tissues exhibiting overexpression of SHIV A protein, it is possible to treat with a candidate substance to determine whether the SHIV A protein activity can be down-regulated in a manner consistent with a therapy.

Treatment of animals with test compounds will involve the administration of the compound, in an appropriate form, to the animal. Administration will be by any route that can be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood, cerebrospinal fluid (CSF) or lymph supply and intratumoral injection.

Determining the effectiveness of a compound in vivo may involve a variety of different criteria. Such criteria include, but are not limited to, survival, reduction of tumor burden or mass, inhibition or prevention of inflammatory response, increased activity level, improvement in immune effector function and improved food intake.

e. Rational Drug Design.

The goal of rational drug design is to produce structural analogs of biologically active polypeptides or compounds with which they interact (agonists, antagonists, inhibitors, peptidomimetics, binding partners, etc.). By creating such analogs, it is possible to fashion drugs which are more active or stable than the natural molecules, which have different susceptibility to alteration or which may affect the function of various other molecules. In one approach, one would generate a three-dimensional structure for SHIV A protein or a fragment thereof. This could be accomplished by x-ray crystallograph, computer modeling or by a combination of both approaches. An alternative approach, “alanine scan,” involves the random replacement of residues throughout molecule with alanine, and the resulting affect on function determined.

It also is possible to isolate a specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have activity as stimulators, inhibitors, agonists, antagonists of SHIV A protein or molecules affected by SHIV A protein function. Such rational drug design may start with lead compounds identified by the present invention, or may start with a lead compound known to be a modulator of HIV-induced apoptosis. By virtue of the availability of cloned SHIV A protein sequences, sufficient amounts of the related proteins can be produced to perform crystallographic studies. In addition, knowledge of the polypeptide sequences permits computer employed predictions of structure-function relationships.

I. Therapeutic Methods.

The present invention deals with the treatment of diseases that result from the increased expression of SHIV A protein. In one embodiment, this protein is seen secreted from macrophages as a result of HIV-1 infection. The secreted product promotes apoptosis in neuronal cells, B-cells and T-cells. The apoptosis in neuronal cells is thought to be a causative factor of HAD. The apoptosis in B and T cells is the major cause of immune cell depletion in systemic HIV which leads to rapid disease progression.

Hence, compositions designed to inhibit the expression or overexpression of SHIV A protein in HIV infected patients will be useful in treating or preventing HAD and in treating systemic HIV progression. Specifically contemplated is the treatment of patients recently exposed to HIV, but not yet tested for, or confirmed to be, HIV positive by standard diagnostic procedures (e.g., neonates from HIV positive mothers, medical personnel expositive to HIV positive blood and the like), patients at risk of exposure to HIV or patients already infected with HIV (i.e., HIV positive patients). Thus, for example, the present invention provides a method of treating HIV infection systemically or HAD specifically in a patient, comprising administering to the patient a composition comprising an effective amount of an antibody or functional fragment thereof which binds to a mammalian SHIV A protein or portion of this protein (e.g., the secreted 6 kDa secreted proapoptotic factor). The composition can also comprise one or more additional agents effective against HIV infection in general, including, but not limited to, HAART therapies. Therapeutic use of antibody to treat HIV infection includes prophylactic use (e.g., for treatment of a patient who may be or who may have been exposed to HIV). Other compositions which inhibit the expression, activity or function of SHIV A protein (e.g., antagonists) also are contemplated for use in such treatment methods.

Additionally, this factor also promotes apoptosis in T-cells, and B-cells. These latter cells play an integral role in disease states such as inflammatory diseases, allergic responses, and the like. Inflammatory disease states include systemic inflammatory conditions and conditions associated locally with the migration and attraction of monocytes, leukocytes and/or neutrophils. Induction of apoptosis using SHIV A protein-based compositions may be useful to ameliorate pathologic inflammatory disease states.

Thus, the present invention contemplates increasing apoptosis in pro-inflammatory tissues such as joint capsules (for disorders such as rheumatoid arthritis, psoriasis, atopic dermatitis), CNS (lupus, encephalitis, guillain-barra), asthma, allergic rhinitis, etc. Such an intervention can be performed by direct injection of the protein, active protein fragment, or through the use of gene therapy. An agonistic antibody to the receptor or small molecule agonist to the receptor may also be used. For asthma or rhinitis an inhaled form may be preferred.

Inflammation may result from infection with pathogenic organisms (including Gram positive bacteria, Gram negative bacteria, viruses, fungi and parasites such as protozoa and helminths) transplant rejection including rejection of solid organs such as kidney liver heart lung or cornea as well as rejection of marrow transplants including graft versus host disease (GVHD) or from localized chronic or acute autoimmune or allergic reactions. Autoimmune diseases include acute glomerulonephritis, rheumatoid or reactive arthritis, chronic glomerulonephritis, inflammatory bowel diseases such as Crohn's disease, ulcerative colitis and necrotizing entercolitis, Addison's disease, Grave's disease, granulocyte transfusion associated syndromes, inflammatory dermatoses such as dermatitis, atopic dermatitis, psoriasis, systemic lupus erthyromatosus (SLE), autoimmune thyroiditis, psoriasis, dermatomyositis, polymyositis, osteoarthritis, osteoporosis, atrophic gastritis, myasthenia gravis, multiple sclerosis, some forms of diabetes, pancreatitis or any other autoimmune states where attack by the subject's own system results in pathological tissue destruction. Any of these disorders may be treated by inducing apoptosis through the action of the SHIV A protein.

Allergic reactions include allergic asthma chronic bronchitis, allergic rhinitis, acute and delayed hypersensitivity. Systemic inflammatory disease states include inflammation associated with trauma, bums, reperfusions following ischemic events (e.g., thrombotic events in heart, brain, intestines or peripheral vasculature, including myocardial infarction and stroke), sepsis, adult respiratory distress syndrome or multiple organ dysfunction syndrome. Inflammatory cell recruitment also occurs in atherosclerotic plaques.

Another significant area in which it may be desirable to induce apoptosis through administration of SHIV A protein based compositions is to treat hyperproliferative disorders such as cancer.

Purified nucleic acid sequences, antisense molecules, PNAs, purified protein, antibodies, antagonists or inhibitors can all be used as pharmaceutical compositions. Delivery of these molecules for therapeutic purposes is further described below. The most appropriate therapy depends on the patient, the specific diagnosis, and the physician who is treating and monitoring the patient's condition.

From the foregoing discussion, it becomes evident that the disease that may be treated, according to the present invention, is limited only by the involvement of SHIV A protein. By involvement, it is not even a requirement that SHIV A protein be mutated or abnormal—the expression or overexpression of this gene may be sufficient to actually affect a therapeutic outcome.

a. Genetic Based Therapies.

One of the therapeutic embodiments contemplated by the present inventors is intervention, at the molecular level, to augment or disrupt SHIV A protein expression. Specifically, the present inventors intend to provide, to a given cell or tissue in patient or subject in need thereof, an expression construct to deliver a therapeutically effective composition to that cell in a functional form. The expression construct may be one which is capable of providing SHIV A protein to the cell; alternatively the expression construct is one which delivers an antisense or other nucleic acid-based construct for the disruption of SHIV A expression in the cell. It is specifically contemplated that the genes disclosed herein will be employed in human therapy, as could any of the gene sequence variants discussed above which would encode the same, or a biologically equivalent polypeptide. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for delivery. The section below on routes contains an extensive list of possible routes. For example, systemic delivery is contemplated. In those cases where the individual being treated has a tumor, a variety of direct, local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

An “individual” as used herein, is a vertebrate, preferably a mammal, more preferably a human. Mammals include research, farm and sport animals, and pets.

b. Protein Therapy.

Another therapy approach is the provision, to a subject, of SHIV A protein polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

In addition, the present invention details methods and compositions for identifying additional modulators of apoptosis such modulators may be used in the therapeutic embodiments of the present invention.

C. Combined Therapy.

In addition to therapies based solely on the delivery of SHIV A protein and related composition, combination therapy is specifically contemplated. In the context of the present invention, it is contemplated that SHIV A protein therapy could be used similarly in conjunction with other agents for inhibiting the proliferation of HIV, other anti inflammatory agents, or those used in the therapy of the disorders enumerated herein.

To achieve the appropriate therapeutic outcome, be it a decrease in viral load, a reduction in tumor size or growth, myelosuppression or any other use for the SHIV A protein discussed herein, using the methods and compositions of the present invention, one would generally contact a “target” cell with a first therapeutic agent that may be a SHIV A protein, SHIV A expression construct, or modulator of SHIV A protein expression and/or activity as defined herein and at least one other therapeutic agent (second therapeutic agent). These compositions would be provided in a combined amount effective to produce the desired therapeutic outcome. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second therapeutic agent.

Alternatively, the first therapeutic agent may precede or follow the other agent treatment by intervals ranging from minutes to weeks. In embodiments where the second therapeutic agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Local delivery of the first therapeutic agent (i.e., the agent based on or derived from the SHIV A protein of the present invention) to patients may be a very efficient method for delivering a therapeutically effective gene to counteract a clinical disease. Similarly, the second therapeutic agent may be directed to a particular affected region of the subject's body. Alternatively, systemic delivery of expression construct and/or the second therapeutic agent may be appropriate in certain circumstances, for example, where extensive metastasis has occurred.

In particularly preferred embodiments, the present invention contemplates therapeutic compositions comprising the SHIV A protein-based compositions in combination with compositions traditionally used in HAART. The compositions traditionally used in HAART would serve as the second therapeutic agent in the above discussion and these agents include but are not limited to protease inhibitors such as saquinavir (e.g., compositions such as INVIRASE and FORTOVASE, Hoffman-LaRoche), indinavir (e.g., CRIXIVAN, Merck & Co.), ritonavir (e.g., NORVIR, Abbott Laboratories), nelfinavir (e.g., VIRACEPT, Pfizer), amprenavir (e.g., AGENERASE, GlaxoSmithKline), a mixture of lopinovir and ritonavir (sold as KALETRA, Abbott Laboratories), fosamprinavir (GlaxoSmithKline), tipranavir (Boehringer Ingelheim) and atazanovir (Bristol-Myers Squibb), nucleoside/nucleotide reverse transcriptase inhibitors such as zidovudine (e.g., RETROVIR, GlaxoSmithKline), didanosine (e.g., VIDEX AND VIDEX-EC, Bristol-Myers Squibb), stavudine (ZERIT, Bristol-Myers Squibb), lamivudine (e.g., EPIVIR, GlaxoSmithKline), zalcitabine (e.g., HIVID, Hoffman-LaRoche), abacavir (e.g., ZIAGEN, GlaxoSmithKline), tenofovir (VIREAD, Gilead Science) and mixtures thereof (e.g., COMBIVIR, a mixture of zidovudine and lamivudine, GlaxoSmithKline; TRIVIVIR a mixture of zidovudine, lamivudine, and abacavir, GlaxoSmithKline), non-nucleoside reverse transcriptase inhibitors such as nevaripine (VIRAMUNE, Boehringer Ingelheim), delavaridine (RESCRIPTOR, Pfizer) and efavirenz (e.g., SUSTIVA, Bristol-Myers Squibb. Another class of inhibitors that are receiving attention as anti-HIV agents are the so-called “entry inhibitors”. Entry inhibitors include agents that inhibit fusion. One such agent is FUZEON (Timeris/Hoffnan LaRoche). FUZEON and other entry inhibitors act by binding to a gp41 on to surface of HIV. Once the gp41 is thus bound, HIV cannot successfully bind with the surface of T-cells, thus preventing the virus from infecting healthy cells. Other apartyl protease inhibitors and nucleoside and non-nucleoside inhibitors of reverse transcriptase, and entry inhibitors also could be used in the combination therapy embodiments of the present invention.

J. Receptor Identification.

Given the identification of proapoptotic factor of the present invention, it will now be possible to identify the endogenous receptor for SHIV A protein and related agents. Once such a receptor is identified it may be employed in various therapeutic applications as well as in the identification of therapeutic compounds through screening assays similar to those described herein above for SHIV A protein.

A cDNA library is prepared, preferably from cells that respond to SHIV A protein. As the receptor may be located on one or more of neuronal cell, T-cells or B-cells, the cDNA library may be prepared from such cells. Radiolabeled SHIV A protein can also be used to identify cell types which express high levels of receptor for SHIV A protein. Pools of transfected clones in the cDNA library are screened for binding of radiolabeled SHIV A protein by autoradiography. Positive pools are successively subfractionated and rescreened until individual positive clones are obtained.

Alternatively, a degenerate PCR strategy may be used in which the sequences of the PCR primers are based on conserved regions of the sequences of known receptors. To increase the chance of isolating an SHIV A protein receptor, the template DNA used in the reaction may be cDNA derived from a cell type responsive to SHIV A protein.

K. Transgenic Animals/Knockout Animals.

In one embodiment of the invention, transgenic animals are produced which contain a functional transgene encoding wild-type or mutant SHIV A protein polypeptides. Transgenic animals expressing SHIV A protein encoding transgenes, recombinant cell lines derived from such animals and transgenic embryos may be useful in methods for screening for and identifying agents that induce or repress function of SHIV A protein. Transgenic animals of the present invention also can be used as models for studying indications of abnormal SHIV A protein expression.

In one embodiment of the invention, a SHIV A protein encoding transgene is introduced into a non-human host to produce a transgenic animal expressing a human SHIV A protein encoding gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al. Proc Natl Acad Sci USA. 82(13):4438 42, 1985; Hammer et al., Nature. 20 26;315(6021):680 3, 1985; Palmiter and Brinster, Cell, 41(2): 343 5, 1985 (which are incorporated herein by reference) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous SHIV A protein by homologous recombination between the transgene and the endogenous gene; or the endogenous gene may be eliminated by deletion as in the preparation of “knock-out” animals. Typically, a SHIV A protein encoding gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which overexpress SHIV A protein or express a mutant form of the polypeptide. Alternatively, the absence of a SHIV A protein in “knock-out” mice permits the study of the effects that loss of SHIV A protein has on a cell in vivo. Knock-out mice also provide a model for the development of SHIV A protein-related abnormalities.

As noted above, transgenic animals and cell lines derived from such animals may find use in certain testing experiments. In this regard, transgenic animals and cell lines capable of expressing wild-type or mutant SHIV A protein may be exposed to test substances. These test substances can be screened for the ability to enhance wild-type SHIV A protein expression and/or function or impair the expression or function of mutant SHIV A protein.

a. Methods of Making Recombinant Cells and Transgenic Animals

As noted above, a particular embodiment of the present invention provides transgenic animals which express or overexpress SHIV A protein. These animals exhibit all the characteristics associated with the pathophysiological features of HAD. Transgenic animals expressing SHIV A protein encoding transgenes, recombinant cell lines derived from such animals and transgenic embryos may be useful in methods for screening for and identifying agents that repress the apoptotic activity of SHIV A proteins or peptides derived thereform and thereby alleviate diseases associated with HIV-1 infection, such as, for example, HAD.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds. Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a SHIV A protein. In particularly preferred embodiments, the transgenic animals express SHIV A protein in macrophages. In still more preferred embodiments, the animals express a 6 kDa fragment of SHIV A protein. In particular embodiments, the SHIV A protein is secreted from those cells and acts of cells in a distal location within the animal. In other preferred embodiments, the transgenic mice express SHIV A protein which is secreted from macrophages of the mice and induces apoptosis of neuronal cells. In other embodiments, the transgenic mice expresss SHIV A protein, which induces apoptosis in T cells and/or B cells.

DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the: dialysis bags, extracted with a 1:1 phenol: chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm is a UV spectrophotometer. For microinjection, DNA concentrations are adjusted to 31 g/ml in:5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); the Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG;Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% CO₂ 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmaker's forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

b. Monitoring Transgene Expression

In order to determine whether the active SHIV A protein has been successful incorporated into the genome of the transgenic animal, a variety of different assays may be performed. Transgenic animals can be identified by analyzing their DNA. For this purpose, when the transgenic animal is a rodent, tail samples (1 to 2 cm) can be removed from three week old animals. DNA from these or other samples can then be prepared and analyzed by Southern blot, PCR, or slot blot to detect transgenic founder (F0) animals and their progeny (F1 and F2).

The various F0, F1 and F2 animals that carry a transgene can be analyzed by any of a variety of techniques, including immunohistology, electron microscopy, and making determinations of total and regional area weights. Immunohistological analysis for the expression of a transgene by using an antibody of appropriate specificity can be performed using known methods. Morphometric analyses to determine regional weights, B and/or T cell counts, and cognitive tests to determine dementia characteristics can be performed using known methods.

In immuno-based analyses, it may be necessary to rely on SHIV A protein-binding antibodies. A general review of antibody production techniques is provided elsewhere in the specification.

Transgene expression may be analysed by measuring mRNA levels in a given cell. Messenger RNA can be isolated by any method known in the art, including, but not limited to, the acid guanidinium thiocyanate-phenol: chloroform extraction method (Chomczynski and Sacchi 1987), from cell lines and tissues of transgenic animals to determine expression levels by Northern blots, RNAse and nuclease protection assays.

Additionally, transgene expression in a given cell also may be determined through a measurement of protein levels of the cell. Protein levels can be measured by any means known in the art, including, but not limited to, western blot analysis, ELISA and radioimmunoassay, using one or more antibodies specific for the protein encoded by the transgene.

For Western blot analysis, protein fractions can be isolated from tissue homogenates and cell lysates and subjected to Western blot analysis as described by, for example, Harlow et al., Antibodies: A Laboratory Manual, (Cold Spring Harbor, N.Y. 1988); Brown et al. (1983); and Tate-Ostroff et al. (1989).

For example, the protein fractions can be denatured in Laemmli sample buffer and electrophoresed on SDS-Polyacrylamide gels. The proteins are then transferred to nitrocellulose filters by electroblotting. The filters are blocked, incubated with primary antibodies, and finally reacted with enzyme conjugated secondary antibodies. Subsequent incubation with the appropriate chromogenic substrate reveals the position of the transgene-encoded proteins.

ELISAs are preferably used in conjunction with the invention. For example, an ELISA assay may be performed where SHIV A protein from a sample is immobilized onto a selected surface, preferably a surface exhibiting a protein affinity such as the wells of a polystyrene microtiter plate. The plate is washed to remove incompletely adsorbed material and the plate is coated with a non-specific protein that is known to be antigenically neutral with regard to the test antibody, such as bovine serum albumin (BSA), casein or solutions of powdered milk. This allows for blocking of nonspecific adsorption sites on the immobilizing surface and thus reduces the background caused by nonspecific binding of antisera onto the surface.

Next, the protein-specific antibody is added to the plate in a manner conducive to immune complex (antigen/antibody) formation. Such conditions preferably include diluting the antisera/antibody with diluents such as BSA bovine gamma globulin (BGG) and phosphate buffered saline (PBS)/Tween®. These added agents also tend to assist in the reduction of nonspecific background the plate is then allowed to incubate for from about 2 to about 4 hr, at temperatures preferably on the order of about 25° to about 27° C. Following incubation, the plate is washed so as to remove non-immunocomplexed material. A preferred washing procedure includes washing with a solution such as PBS/Tween®, or borate buffer.

Following formation of specific immunocomplexes between the sample and antibody, and subsequent washing, the occurrence and amount of immunocomplex formation may be determined by subjecting the plate to a second antibody probe, the second antibody having specificity for the first (usually the Fc portion of the first is the target). To provide a detecting means, the second antibody will preferably have an associated enzyme that will generate a color development upon incubating with an appropriate chromogenic substrate. Thus, for example one will desire to contact and incubate the antibody-bound surface with a urease or peroxidase-conjugated anti-human IgG for a period of time and under conditions which factor the development of immunocomplex formation (e.g., incubation for 2 hr at room temperature in a PBS-containing solution such as PBS/Tween®.

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H2O2 in the case of peroxidase as the enzyme label. Quantitation is then achieved by measuring the degree of color generation, e.g., using a visible spectrum spectrophotometer. Variations on this assay, as well as completely different assays (radioimmunprecipitation, immunoaffinity chromatograph, Western blot) also are contemplated as part of the present invention.

Other immunoassays encompassed by the present invention include, but are not limited to those described in U.S. Pat. No. 4,367,110 (double monoclonal antibody sandwich assay) and U.S. Pat. No. 4,452,901 (Western blot). Other assays include immunoprecipitation of labeled ligands and immunocytochemistry, both in vitro and in vivo.

C. Methods of Using Recombinant Cells and Transgenic Animals

The transgenic animals of the present invention include those which have a substantially increased probability of spontaneously developing dementia, and in particular HAD, when compared with non-transgenic littermates. A “substantially increased” probability of spontaneously developing nephropathy means that, a statistically significant increase of measurable symptoms of nephropathy and kidney dysfunction is observed when comparing the transgenic animal with non-transgenic littermates.

The transgenic animals of the present invention are produced with transgenes which comprise a coding region that encodes a gene product which induces apoptosis in neuronal cells of the animal and manifests signs of HAD.

As used herein, such a “signal” indicates any stimulus, mechanical or chemical, which results in measurable symptoms of HAD. The neuropathological characteristics of HAD include but are not limited to, widespread reactive astrocytosis, myelin pallor, and infiltration predominantly by monocytoid cells, including blood-derived macrophages, resident microglia and multinucleated giant cells (Lipson and Gendelman, N Eng J Med 332:934, 1995). Currently, no animal models of HAD are known, and as such, even if the transgenic mice exhibit slight manifestations of dementia, the mice of the present invention provide an in vivo model that may be used for further study HAD.

Coding regions for use in constructing the transgenic mice include the coding region for SHIV A protein, however, it is contemplated that transgenic mice also may be constructed using coding regions for one or more of the other accessory proteins of HIV-1. The coding regions may encode a complete polypeptide, or a fragment thereof, as long as the desired function of the polypeptide is retained, i.e., the SHIV A protein can disrupt normal neuronal cell, T cell and/or B cell viability and cause apoptosis. The coding regions for use in constructing the transgenes of the present invention further include those containing mutations, including silent mutations, mutations resulting in a more active protein, mutations that result in a constitutively active protein, and mutations resulting in a protein with reduced activity.

The transgenic mice of the present invention has a variety of different uses. First, by creating an animal model in which the SHIV A protein is expressed and constantly activated, the present inventors have provided a living “vessel” in which the function of SHIV A protein may be further dissected. For example, provision of various forms of SHIV A protein—deletion mutants, substitution mutants, insertion mutants, fragments and wild-type proteins—labeled or unlabeled, will permit numerous studies on HAD that were not previously possible. Additionally, the animals provide a vehicle for testing non-SHIV A protein related drugs that may ameliorate HAD. Thus, the transgenic mouse provides a novel model for the study of HIV-1 associated disorders. This model could be exploited by treating the animal with compounds that potentially inhibit the in vivo action of SHIV A protein and treat HIV-related dementia.

L. Pharmaceutical Compositions.

Where clinical applications are contemplated, it will be necessary to prepare the viral expression vectors, antibodies, peptides, nucleic acids and other compositions identified by the present invention as pharmaceutical compositions, i.e., in a form appropriate for in vivo applications. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramusclar, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

In the clinical setting an “effective amount” is an amount sufficient to effect beneficial or desired clinical results. An effective amount can be administered in one or more doses. In terms of treatment, an “effective amount” of polynucleotide, and/or polypeptide is an amount sufficient to palliate, ameliorate, stabilize, reverse, slow or delay the progression of apoptosis-associated disease states or otherwise reduce the pathological consequences of the disease. The effective amount is generally determined by the physician on a case-by-case basis and is within the skill of one in the art. Several factors are typically taken into account when determining, an appropriate dosage. These factors include age, sex and weight of the patient, the condition being treated, the severity of the condition and the form of the antibody being administered. For instance, in embodiments in which the antibody compositions of the present invention are being therapeutically administered, it is likely the concentration of a single chain antibody need not be as high as that of native antibodies in order to be therapeutically effective.

“Unit dose” is defined as a discrete amount of a therapeutic composition dispersed in a suitable carrier. For example, where polypeptides are being administered parenterally, SHIV A protein polypeptide compositions are generally injected in doses ranging from 1 μg/kg to 100 mg/kg body weight/day, preferably at doses ranging from 0.1 mg/kg to about 50 mg/kg body weight/day. Parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435 1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

In a preferred embodiment, the present invention is directed at treatment of human disorders that are caused by the presence of SHIV A (as in the case of HAD, which results from apoptosis of neuronal cells caused by SHIV A or a fragment thereof), or may be alleviated by administering SHIV A (e.g., disorders that could benefit from apoptosis of aberrant cells, such as, hyperproliferative disorders or inflammatory diseases). A variety of different routes of administration are contemplated. For example, a classic and typical therapy will involve direct, injection of a discrete area of inflammation. In the case of a tumor, the discrete tumor mass may be injected. The injections may be single or multiple; where multiple, injections are made at about 1 cm spacings across the accessible surface of the tumor. Alternatively, targeting the tumor vasculature by direct, local or regional intra-arterial injection are contemplated. The lymphatic systems, including regional lymph nodes, present another likely target for delivery. Further, systemic injection may be preferred.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

### M. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The following materials and methods were used in specific exemplary embodiments of the present invention.

Human macrophage hybridomas, Bosc cells and SH-SY5Y cells: Human macrophage hybridomas were obtained by fusing macrophages (obtained by allowing monocytes to mature into macrophages in Teflon bag cultures) with a hypoxanthine-guanine phosphoribosyltransferase-deficient promonocytic line (U937) as previously described (Sperber et al., J Immunol Methods 129:31, 1990). One clone, 43, was uniformly infected and characterized, with different strains of HIV-1 (43_{HIV})(Yoo, et al., J Immunol 157:1313, 1996; Polyak et al., J Immunol 159:2177, 1997; Chen et al., J Immunology 161:4257, 1998; Rakoff-Nahoum et al., Journal of Immunology 167:2331, 2001). Bosc cells are derived from L23T cells and are a gift of Dr K Horvath (Immunobiology Institute, Mount Sinai School of Medicine) (Pear et al., Proc Natl Acad Sci USA 90:8392, 1993). The SH-SY5Y cells were purchased from the American Tissue and Culture Collection (Rockville, Md.) (Ross et al., J National Cancer Institute 71: 741, 1983). The IMR, H-9, THB cells, the LAZ and Ramos B cell lines, the respiratory epithelial cell lines A549 and BEAS-2B, the gastrointestinal epithelial cell lines HT-29, T-84 and Caco-2, the uterine epithelial cell line Ishikowa, the RR-MRC-5 fibroblast cell line, the astrocytoma cell lines (CCF-STTG1, SW1088) were purchased from the American Tissue and Culture Collection (ATCC) (Manassas, Va.). The primary fetal neurons were purchased from the ATCC and CLONETICS (Walkersville, Md.). The primary T cells were isolated from buffy coats by resetting using sheep red cells as previously described (Yoo et al., J Immunol 1996; 157:1313).

Peripheral Blood mononuclear cells, CD4+ and CD8+ T cells and B cells: Peripheral blood mononuclear cells (PBMC) were separated from buffy coats obtained from normal healthy volunteers by Ficoll-Hypaque (Pharmacia, Piscataway, N.J.) density gradient centrifugation. The cells were washed three times with sterile PBS and resuspended in RPMI 1640 (Life Technologies, Grand Island, N.Y.) supplemented with 10% fetal calf serum (Life Technologies), 2 mM L-glutamine and 1% penicillin/streptomycin (Life Technologies) henceforth called complete medium (CM) (Sperber et al., J Immunol Methods 129:31, 1990). CD4+ and CD8+ T cells and B cells populations were isolated and purified by RosetteSep™ (Stem Cell Technologies, Vancouver, BC, Canada). RosetteSep™ is a rapid, easy cell separation kit for the isolation of highly purified CD4+ and CD8+ T and B cells from whole blood. Whole blood is added to a RosetteSepm cocktail and cells are cross linked with tetramer complexes. The cells are then incubated at room temperature, layered over Ficoll-Hypaque, centrifuged for 20 minutes and the enriched CD4+ and CD8+ T and B cells isolated (Bader et al., Transplant 20:79, 1999).

Spleen cells: Balb C mice used for the spleen cultures were purchased from Charles River Laboratories (Wilmington, Mass.). Spleen cells were taken and treated with 0.17 M Tris-NH₄Cl RBC lysis buffer to remove RBC by previously described methods (Lopez et al., J Interferon Cytokine Res 21:763, 2001). The isolated spleen cells were used in the apoptosis assay.

Acetone Precipitation: Acetone precipitation was used to isolate the proapoptotic factor from crude supernatants from the 43 and 43_{HIV} cell lines. Acetone (Sigma, St Louis, Mo.) was chilled in an ice-salt bath to attain a temperature below 0° C. Proteins were fractionated from 43 and 43_{HIV} supernatants by precipitation in 95% (v/v) acetone. The precipitated proteins were collected by centrifugation, and the residual acetone in the precipitates was removed by vacuum centrifugation in a Speed-Vac (Savant) (Chen et al., J Immunology 161:4257, 1998).

Reverse phase HPLC analysis: Reverse phase HPLC was performed on the acetone precipitate from the 43 and 43_{HIV} cells using a C18 (4.6×250 mm) column. Elution of bound proteins was developed using a linear gradient of 0.1% (v/v) trifluoroacetic acid. A gradient of 60 ml was developed at a flow rate of 1 ml/min. Elution profiles were monitored at an absorbance of 215 nm. Solvent in the protein-containing fractions was removed by vacuum centrifugation in a Speed-Vac (Savant, Piscataway, N.J.) (Chen et al., J Immunology 161:4257, 1998).

Anion Exchange Chromatography: Anion exchange chromatography was performed with a Mono-Q HR5/5 (5×50 mm) column on an FLPC system (Pharmacia). The elution gradient was developed using 20 mM Tris-HCl, pH 7.5 (buffer A), and 1 M NaCl in buffer A (buffer B) at a flow rate of 1 ml/minute. Samples were prepared for anion exchange chromatography by exhaustive dialysis in buffer A. The protein elution profile was monitored by absorbance at 280 nm (Chen et al., J Immunology 161:4257, 1998).

Generation of Antibodies: To clone the proapoptotic factor, anti-proapoptotic factor monoclonal and polyclonal antibodies were generated by techniques previously established (Sperber et al., Am Rev Respir Dis. 146:1589, 1992). Monoclonal antibodies were generated by boosting 2 Balb C mice immunized with HPLC fractionated supernatant from the 43_{HIV} cell line as previously described, followed by fusion to the non-IgG secreting mouse myeloma cell line SP2/0 (Sperber et al., Am Rev Respir Dis. 146:1589, 1992). The monoclonal antibodies were screened by immunoblot and by inhibition of apoptosis in the bioassay using PBMC as targets cells. For the immunoblot screening crude Fraction 5 of the 43_{HIV} supernatant was applied to nitrocellulose membranes using a Bio-dot apparatus (Bio-Rad, Richmond, Calif.) that permits application of uniform dots. Test supernatants from the fusion were added to the Fraction 5 containing nitrocellulose paper in 96 well microtiter plates for 2 hours at 25° C. followed by 5 PBS washes and the addition of horseradish peroxidase conjugated goat anti-mouse antibody (Life Sciences, Burlingame, Calif.) for 2 hours at 25° C. Irrelevant murine monoclonal antibodies of all isotypes will be used as specificity controls in each assay. After extensive PBS washing, substrate (3,3′-diaminobenzidine tetrahydrochloride; Pierce Chemical Company, Rockford, Ill.) was added and the plates read for the appearance of a blue color at 30 minutes, indicating a positive result. Since many monoclonal antibodies do not react in western blots, screens using inhibition of apoptosis in the bioassay also were used. In addition, rabbit anti-proapoptotic factor antibodies were generated by intramuscularly injecting two rabbits (Mount Sinai Animal Facilities) with fractionated supernatant from the 43_{HIV} cell line with complete Freund's adjuvant (Sigma) with two booster injections (Sperber et al., Am Rev RespirDis. 146:1589, 1992). Polyclonal anti-sera was prepared and pooled. IgG was purified by Protein A-Sepharose (Pharmacia) as previously described (Sperber et al., Am Rev Respir Dis. 146:1589, 1992). Ouchterlony immunodiffusion in gels was used to test anti-serum generated against the proapoptotic fractions. Immunoelectrophoresis was performed in 2% sodium dodecylsulfate (Sigma) that contained 0.01 mL of ethylenediaminetetraacetic acid (Sigma). Rabbit serum against purified proapoptotic factor developed as a single band in Ouchterlony immunodiffusion. Rabbit antibodies were also assayed by their ability to block apoptosis in vitro using Annexin V staining.

Annexin V staining apoptosis assay: FITC-labeled Annexin V, a phospholipid binding protein of the Annexin family (Fadak et al., J Immunol 148:2207, 1992; Koopman et al., Blood 84:1415, 1994), was used to measure apoptosis using a commercially available kit (Coulter, Hialeah, FLA). After incubating supernatants containing concentrations of the SHIV A protein with PBMC, T cells and B cells, the cell samples were washed with ice cold PBS followed by centrifugation at 500×g at 4° C. The cells were incubated with Annexin V FITC at room temperature for 10 minutes in the dark. The cells were then analyzed by flow cytometry to measure the Annexin V+population, gating on the live cells (Chen et al., J Immunology 161:4257, 1998).

Lamda ZAP EcoR I/Xho I 43HIV cDNA Library: A 43_{HIV} library was synthesized using the ZAP-cDNA synthesis method (Stratagene, La Jolla, Calif.) (Short et al., Nucleic Acid Res 16:7583, 1989). The linker-primer was designed with a GAGA sequence to protect the Xho I restriction site and an 18-base ply (dT) sequence. The restriction site allows the finished cDNA to be inserted onto the vector unidirectionally in the sense orientation with respect to the lacZ promoter. The linker-primer is a 50-base oligonucleotide with the sequence of SEQ ID NO:4, i.e., the following sequence:

| | | |
|---|---|---|
| 5′ GAGAGAGAGAGAGAGAGAAACTAGTCTCGAG(T)₁₈ 3′ | GAGA sequence | Xho I |

The adaptors are comprised of 8- and 13-mer oligonucleotides that are complementary to each other and have an EcoR I cohesive end. The adaptors have the following sequence:

| | |
|---|---|
| 5′ AATTCGGCACGAG 3′ | (SEQ ID NO:5) |
| 3′ GCCGTGTC 5′. | (SEQ ID NO:6) |

The amplified library was grown in XL1-Blue MRF′ strain. Two different helper phages were used with the ZAP Express library, the ExAssist™ (Stratagene) interference-resistant helper phage and the 408 helper phage. To screen for the proapoptotic factor expressing clones, E coli were grown on agar and the colonies harvested onto 10 mM isopropyl-β-D-thiogalactopyranosise (ITPG) soaked nitrocellulose filters for 16 hours overnight. The membranes were washed 3 times in PBS and western blotted with either rabbit or mouse anti-proapoptotic antibodies followed by either goat anti-rabbit or anti-mouse labeled horse radish labeled secondary antibodies (Pharmacia) and developed using a commercially available ECL kit (Dupont, Wilmington, Del.) (Rakoff-Nahoum et al., Journal of Immunology 167:2331, 2001).

Western Blot Analysis: Cells for western blot analysis were lyzed using buffer containing PMSF (100 mM), apoprotinin (10 mg/ml), leupeptin (10 mg/ml), iodoacteamide (1.8 mg/ml) and 0.1% Triton X (Sigma). The lysates were resolved on a 10% SDS-PAGE gel, transferred onto a nitrocellulose membrane, blocked with 5% milk in PBS at room temperature for 60 minutes, then incubated with the anti-proapoptotic antibodies Abs (37°) for 43, 43_{HIV}, Bosc cells and E coli and with anti-caspase 3 and anti PARP antibodies (PharMingen, San Diego, Calif.) for the SH-SY5Y cells at 4° C. overnight, the rabbit anti-Caspase 9 for SH-SYSY, IMR, and MC-IXC cells and Apaf-1, Bad, Bax, Bcl-2, Bcl-xL, Bruce, CAS, hILP/XIAP, Mcl-1, Nip1 and p53 antibodies (BD Biosciences) for SH-SY5Y, IMR, MC-IXC, THB, and H-9 cells and with rabbit and mouse anti-SHIV A antibodies for E. coli at 4° C. overnight. A secondary horseradish labeled goat anti-mouse Ig or goat anti-rabbit (Tago) was then added at 25° C. for 2 hours and the blot developed by chemiluminescence utilizing a commercially available ECL kit (Rakoff-Nahoum et al., Journal of Immunology 167:2331, 2001) (Dupont).

Purification of 6000 d peptide: The 6000 d peptide was purified from the supernatant of E. coli expressing the SHIV A protein. To this end, 2 liters of bacterial supernatant was concentrated by lyophilization, the concentrated material was resuspended in 100 mM NaCl, 20 mM Tris HCl pH 7.5 and loaded it onto a DEAE sepharose column (Pharmacia). Increasing the salt concentration from 100 mM to 1 M NaCl eluted fractions. The isolated fractions were run on a 10% polyacrylamide gel, transferred onto nitrocellulose paper and western blotted with the rabbit anti-SHIV A antibodies. The western blot positive fractions were then silver stained to ascertain the purity of the protein separation.

Transfection with FL14676485: The 43 and Bosc cells were transiently transfected with a bacterial plasmid containing the FL14676485 cDNA clone using CaPO₄, Superfect (Qiagen, Valencia, Calif.) or DEAE-dextran (Rakoff-Nahoum et al., Journal of Immunology 167:2331, 2001). The bacteria containing the FL14676485 cDNA clone were cultured, ethanol precipitated, extracted, and then centrifuged and re-suspended in TBS. For CaPO₄, a DNA/CaCl₂/H2O mixture (500 μl) containing 20 μg of expressed FL14676485 plasmid, single stranded DNA carrier, 438 μl of H20 and 62 μl 2M CaCl₂ was added to Hank's balanced salt solution and incubated with either Bosc cells or 43 cells for 5 hours. The 43 and Bosc cells were washed and the medium replaced. The cells were harvested at 48 to 60 hours post-transfection.

For Superfect (Qiagen), 5 μg of FL14676485 DNA was dissolved in TE buffer pH 7.4 with media that contains no serum proteins or antibiotics to a total volume of 150 μl. Thirty μl of Superfect (Qiagen) reagent was added to the DNA solution, mixed and then added to the Bosc and 43 cells for 10 minutes at 25° C. to allow for transfection complex formation. The cells were then washed once with 4 ml of PBS and then 1 ml of CM was added. The cells were incubated with the transfection complex for 3 hours at 37° C. The medium was removed and the cells washed in PBS. Fresh CM was added and the cell supernatants assayed at 48 and 60 hours for apoptotic activity.

For the DEAE-dextran method, the re-suspended DNA was added to 10 mg/ml of DEAE-dextran (Sigma) and incubated with the 43 and Bosc cells for 4 hours at 37° C. After aspirating the DEAE-dextran, the cells were shocked by adding 5 ml of 10% DMSO (Sigma) in PBS for 1 minute at room temperature, washed with sterile PBS and re-suspended in CM for 48 hours at 37° C. In some experiments, the 43 and Bosc cells were treated with CaP0₄ alone, Superfect alone or DEAE-dextran alone without the FL14767485 cDNA clone while in other experiments the cells were treated with CaP0₄, Superfect (Qiagen), and DEAE-dextran and jellyfish green fluorescent protein (Promega, Milwaukee, Wis.) to assess the efficacy of transfection.

THB and SH-SY5Y cells were transfected with human Bcl-2 cDNA clone obtained Human BioTrack cDNA clone collection (Huntsville Ala.) using Superfect as previously described (Sperber et al., J Immunol 2003; 170:1566).

PCR: RNA was extracted from 43 and 43_{HIV} using acid quanidium thiocynate/phenol/chloroform as described previously (RNAzol, Linnai, Tex.) (Wang et al., J Immunol 152:3842, 1994). Known quantities of RNA were mixed with 1 μg total cellular RNA and reverse transcribed at 37° C. for 60 minutes in 20 μl of buffer containing 10 mM Tris (Sigma), pH 8.3; 50 mM KCl (Sigma); 5 mM MgCl₂ (Sigma); 1 mM each of dATP, dCTP, dGTP, and dTTP (Sigma) and 20 U RNase inhibitor (Promega), 0.1 μg oligo (dT)₁₅ (Boehringer Mannheim, Indianapolis, Ind.) and 50 U MLV Reverse Transcriptase (Bethesda Research Laboratories, Bethesda, Md.). The PCR for FL14676485 was performed using the upstream primer 5′-TAGAAAACTGGGAAAAAGACATTA-3′ (SEQ ID NO:7) and the downstream primer 5′-TTGGCAACACGGGATTA-3′ (SEQ ID NO:8) for 40 cycles at 51.7° C. for 1 minute, at 50° C. for 1 minute, and at 68° C. for 3 minutes with a 20 second elongation step per cycle. Reactions were stopped by heat inactivation for 10 minutes at 95° C., annealed for 2.5 minutes and extended at 65° C. Negative controls were performed omitting RNA from the cDNA synthesis and specific amplification. PCR products were separated in a 2% NuSieve agarose (FMC, Rockland, Me.) or a 5% polyacrylamide gel (Rakoff-Nahoum et al., Journal of Immunology 167:2331, 2001).

In another exemplary real time PCR, the same primers for the SHIV A cDNA (Integrated DNA Technologies, Inc., Coralville, Iowa) was performed for 40 cycles at 94° C. for 30 seconds, 55° C. for 30 seconds, 72° C. for 45 seconds in a CYBR green containing buffer (BRL, Roche, Indianapolis, Ind.) using a “one tube” RT-PCR according to the manufacturer's recommendations. Standard curves were produced from triplicate reactions for SHIV A with dilutions from RNA from transformed E coli that express SHIV A. Omitting RNA from the cDNA synthesis and specific amplification performed the negative controls. PCR products were checked for the amplification of single, expected bands in a 2% NuSieve agarose (FMC, Rockland, Me.) or a 5% agarose gel (Peters et al., J Immunol Methods 2003; 275:213).

Immunofluorescence: Frozen sections of brain tissue and lymph tissue were obtained from the Manhattan AIDS Brain bank. The cells were fixed with 1% paraformaldehyde and then stained with murine anti-proapoptotic antibodies as the primary antibodies and fluorescein conjugated F (ab)′2 goat anti-mouse IgG (Tago, Burlingame, Calif.) as a secondary antibody mAb W6/32 (anti-class I) and IgG₁ isotype controls as positive respectively. The sections were mounted with Immun-mount (Shandon, Pittsburgh, Pa.) before being viewed by a Leica fluorvent laser-scanning confocal microscope (Leica, Deerfield, Ill.) at s step position of 1 μm on the x-y or x-z axis (Polyak et al., J Immunol 159:2177, 1997).

Intracytoplasmic staining for p24, Bcl-2, SHIV A, and activated Caspase-3: Forty-three cells infected with HIV-1BaL (AIDS Research, Reference and Reagent Program, Bethesda, Md.), the dual tropic isolates HIV_{-187.9} (AIDS Research, Reference and Reagent Program) and HIV produced by 43_{HIV} cells 5 weeks after infection were stained intracytoplasmically for the presence of p24 and SHIV A at weekly intervals after HIV infection. The 43_{HIV} cells were permeabilized with 70% ethanol, washed 3 times with PBS and then stained with FITC-labeled anti-SHIV A antibodies (Chen et al., J Immunol 1998; 161:4257) and PE-labeled anti-p24 mAbs (BD-Biosciences, San Diego, Calif.) for 45 minutes at 4° C. The cells were analyzed by flow cytometry, gating on-live cells (Sperber et al., J Immunol 2003; 170:1566, Chen et al., J Immunol 1998; 161:4257). In other experiments, primary neurons were stained intracytoplasmically with anti-Caspase-3 mAbs (BD-Biosciences) directed against activated Caspase-3 and analyzed by flow cytometry as described above. SH-SY5Y cells transfected with Bcl-2 will be stained intracytoplasmically with FITC labeled anti-Bcl-2 antibodies (BD Biosciences) and then analyzed by flow cytometry as described above.

Tropism of progeny virus produced by 43HIV: U87.CD4.CCR5 and U.87.CD4.CXCR4 cells (Bjomdal et al., J Virol 1997; 71: 7478) were obtained from the AIDS Research Reference and Reagent Program, cultured in DMEM, 15% FCS, supplemented with 1 μg/ml puromycin, 300 μg/ml G418, glutamine, and 1% penicillin/streptomycin (Sigma, St Louis, Mo.), and then infected with HIV-1 produced by 43HIV at weekly intervals after infection with HIV-1BaL. The HIV used to infect the U.87.CD4.CCR5 and U87.CD4.CXCR4 cells was standardized to contain equivalent amounts of virus based on reverse transcriptase activity (80,000 cpm/ml) (Rakoff-Nahoum et al., J Immunol 2001; 167:2331; Chen et al., J Immunol 1998; 161:4257; Polyak et al., J Immunol 1997; 159:2177; Yoo et al., J Immunol 1996; 157:1313; Sperber et al., AIDS Res Human Retroviruses 1993; Sperber et al., J Immunol Methods 1990; 9:657; Sperber et al., J Immunol 2003; 170:1566). HIV-1 infection was determined by measuring the presence of p24 in the culture supernatant by ELISA (Dupont, Willington, Del.) 7 days after infection (Rakoff-Nahoum et al., J Immunol 2001; 167:2331; Chen et al. J Immunol 1998; 161:4257; Polyak et al., J Immunol 1997; 159:2177; Yoo et al., J Immunol 1996; 157:1313; Sperber et al., AIDS Res Human Retroviruses 1993; Sperber et al., J Immunol Methods 1990; 9:657; Sperber et al., J Immunol 2003; 170:1566).

Northern blot analysis: Multiple human tissues were probed by northern blot analysis including heart, brain, placenta, lung, liver, skeletal muscle, kidney, pancreas, testes, ovary, small intestinal, colon, peripheral blood leukocytes, lymph nodes, bone marrow, fetal liver and thymus (CLONTECH Master Blot, Palo Alto, Calif.) for the presence of the mRNA for the SHIV A protein. The RNA on the Master Blot was probed for the FL14676485 gene that encodes the SHIV A protein using a DNA probe. ³²p labeled anti-sense transcripts were generated following linearization. The Master Blot was washed in 2×SSC (0.3 M NaCl in 0.03 M sodium citrate) in 0.1% SDS-at 42° C. followed by exposure to X-Ray film for 7 days (Sperber et al., AIDS Res Hum Retroviruses 1993; 9: 91).

Generation of a fusion protein: The FL14676485 cDNA clone was amplified using the polymerase chain reaction and different primers to clone in frame into EcoRI/XhoI-cut pET28a followed by sequencing with Sequenase kits (CLONTECH). BL21 (DE3) pLysS bacteria (Novagen, San Francisco, Calif.), transformed with these plasmids, were grown in LB media to OD=0.6 at 37° C., and then grown for an additional hour at 30° C. in LB plus 1 mM isopropyl-1-thio-b-D-galactopyranoside to induce protein expression. The pelleted bacteria were suspended in 3 ml/g of bacterial pellet in buffer A (50 mM Tris-HCl, pH 8.0, 1 mM EDTA, 100 mM NaCl, 1 mM sodium fluoride, 0.5 mM sodium vanadate, 2 μg/ml each of Transylol, leupeptin, antipain, pepstatin A, and 1 mM phenylmethylsulfonylurea fluoride, Sigma) and lyzed by 3 to 5 cycles of freezing and thawing. Debris was removed by centrifugation for 10 minutes at 3000-×g (4° C.). This was followed by the sequential addition of 4 mg of deoxycholic acid/g of bacterial pellet (while stirring, until viscous), MgS04 to a final concentration of 5 mM and then 300 units of Benzonase (Sigma). After 30 minutes of incubation on ice the lysate was checked for loss of viscosity using a Pasteur pipette. Debris was then pelleted by centrifugation at 16,000×g for 15 minutes at 4° C. The purity of the proteins was assessed by electrophoresis using 6% SDS-polyacrylamide gels followed by Coomassie Blue (Sigma) staining. Protein concentrations were determined by spectrophotometry at 280 nm.

Calcium influx: Indo-1, a fluorescent indicator with spectral properties that change with the binding of Ca²⁺, was used to measure changes in intracellular calcium concentrations. The neuronal cell lines (10⁶) and primary neurons (10⁶) were incubated with Indo-1 acetomethoxy ester (Molecular Probes, Eugene, Oreg.) for 1 hour at 37° C. After loading, the cells were washed once in PBS and maintained at 25° C. for 5 minutes in PBS plus 1 mM Ca²⁺ and Mg²⁺. Indo-1 fluorescence analysis was performed on an ELITE flow cytometer (Coulter). Ionomycin (Molecular Probes) was used as the positive control. The laser was adjusted for UV excitation and the light scatter was measured at 90 degrees using the UV line. Short signal fluorescence was measured at 395-band pass and the long signal was measured at 525-band pass. The long/free and short/band signal ratios were measured directly (Yoo et al., J Immunol 1996; 157:1313).

Cytochrome c Release: Cytochrome c was detected in the cytoplasm of SHUVA treated cells using a commercially available kit (Oncogene, San Diego, Calif.). Cells (5×10⁷) were treated with the SHIV A fusion protein for 16 hours at 37° C., followed by centrifugation at 600-× g for 5 minutes at 40° C. The cells were washed with ice cold PBS and the pellet was centrifuged at 600-× g for 5 minutes at 4° C. The cells were re-suspended in 1× Cytosol Extraction Buffer Mix containing DTT and protease inhibitors and then incubated on ice for 10 minutes. The cells were then homogenized in an ice-cold tissue blender for 50 passes. Examining cells under a microscope checked the efficiency of the homogenization. A shiny ring around the nuclei of 80% of the cells was observed and the homogenate was transferred into a 1.5 ml tube and centrifuged at 700-x g for 10 minutes at 4° C. The supernatant was transferred to a fresh 1.5 ml tube and centrifuged at 10,000×g for 30 minutes at 4° C. This was the cytosolic fraction. The pellet was re-suspended in 0.1 ml Mitochondria Extraction Buffer Mix containing DTT and protease inhibitors and then vortexed for 10 seconds and saved as the Mitochondria Fraction. Ten micrograms of the cytosolic and mitochondrial fractions were subjected to western blot analysis using murine anti-cytochrome c antibodies as described above (Gosham et al., J Biol Chem 1991; 266:2134).

Caspase-3 ELISA: Activated human Caspase-3 was measured by Ag capture ELISA in cells treated with SHIV A using a commercially available kit (R&D, Minneapolis, Minn.).

Nitric Oxide and glutathione production: Nitric Oxide and intracellular glutathione levels were measured in the SY-SY5Y, IMR, MC-IXC, THB and H-9 cells using commercially available kits from iNtRON Biotechnology (Surrey, UK) and Oncogene (San Diego, Calif.) respectively, according to the manufacturer's recommendations.

N-Acetyl cysteine treatment: N-Acetyl cysteine (Sigma) (10⁻⁴-10⁻¹ M) was pre-incubated with SH-SY5Y, IMR, MC-IXC, H-9 and THB cells for 1 hour. One hundred micrograms/ml of SHIV A were added for 16 hours and apoptosis evaluated using a commercially available Caspase 3 ELISA kit as described above.

### Example 2

### Characterization of Proapoptotic Factor

Previously, proapoptotic activity was demonstrated from fractionated supernatant from the chronically HIV-1 infected cell line, 43_{HIV} (Chen et al., J Immunology 161:4257, 1998). Proapoptotic activity could not be precipitated with acetone at a concentration lower than 80% saturation, a characteristic observed with smaller peptides. Results from peptide binding to anion-exchange matrices at different pH indicated that the pI of the pro-apoptotic activity was between 6.5 and 7.0. When active fractions were electrophoresed on a 10% SDS-PAGE gel, a band corresponding to a Mr of 6000 Da was detected. Furthermore, active fractions from 43_{HIV} were electrophoresed on a non-denaturing SDS-PAGE gel and proapoptotic activity was electroeluted from gel slices corresponding to a Mr of less than 10,000 Da.

Although two proapoptotic fractions were identified from 43_{HIV} supernatant, i.e., Fractions 5 and 6, only Fraction 5 was characterized here. Fraction 5 was further characterized from the 43_{HIV} supernatant by reverse phase HPLC analysis. HLPC elution profiles of Fractions 5 were compared and revealed that 8 peaks were present in Fraction 5 from 43_{HIV} supernatant but not in the uninfected 43 supernatant. None of the fractions from the HPLC appeared to have activity in the apoptosis assay measuring Annexin V staining in bystander T cells. It is possible that biological activity was lost during the isolation procedure. The inventors then developed a panel of murine monoclonal and rabbit polyclonal antibodies by immunizing mice and rabbits with the 8 unique sub-fractions of Fraction 5. In these studies, the inventors first screened by dot blot reaction and then attempted to block apoptosis induced by crude 43_{HIV} Fraction 5 and found that antibodies directed against Sub-fractions 2, 5, and 8 of Fraction 5 of 43_{HIV} were all capable of blocking apoptosis in unstimulated target T cells.

Using the panel of antibodies directed against Sub-fractions 2, 5, and 8 of Fraction 5, the proapoptotic factor of the present invention was identified by screening a cDNA library (λZapII) generated from HIV-1 infected 43 cells. The 43_{HIV} cDNA library was expressed in E. coli and in the screening process the expressed proteins were transferred onto nitrocellulose filters. The expressed proteins were “lifted” off onto nitrocellulose membranes and the membranes were blocked with milk proteins (Carnation milk 10% w/v) and subsequently incubated with the rabbit and mouse anti-apoptotic factor antibodies (10 μg/ml) or a pre-immune serum/mAb negative control. Antibody bound to the expressed proteins was detected by incubation with either goat anti-rabbit IgG or goat anti-mouse IgG conjugated with horseradish peroxidase. Initially, the integrity of the library was established by screening with antibodies directed against proteins expressed in 43_{HIV} cells (e.g., IL-10, IL-8, IL-6 and HIV viral products.). The library was first screened by western blot with the rabbit polyclonal antibodies and then screened the positive clones by western blotting with the murine monoclonal antibodies. The identified plaques were picked, re-grown, re-screened, expanded, and eventually a pure clone was obtained. The cDNA isolated from this clone was sequenced at the Mount Sinai DNA core sequencing facility and found to be the recently described FL14767485 gene that encodes for the hypothetical protein FLJ21980 (FIG. 1A).

The initially described pro-apoptotic factor had a molecular weight of 6000 d while the SHIV A protein has a molecular weight of 66 kDa (Chen et al., J Immunology 161:4257, 1998). To reconcile this discrepancy, western analysis was performed on lysates and supernatants from E coli expressing the SHIV A protein (FIG. 1B). In the lysate, a 66 kDa band was identified in accord with the molecular weight of the fill length SHIV A protein, while in the supernatant a doublet of 46 kDa and a 6000 d band consistent with the previously described factor was identified (FIG. 1B). In the initial characterization of pro-apoptotic activity from the 43_{HIV} supernatant, active pro-apoptotic fractions were isolated with 95% acetone. Ninety-five percent acetone treatment eliminates proteins with molecular weights greater 10 kDa including the 46 kDa doublet from the SHIV A protein.

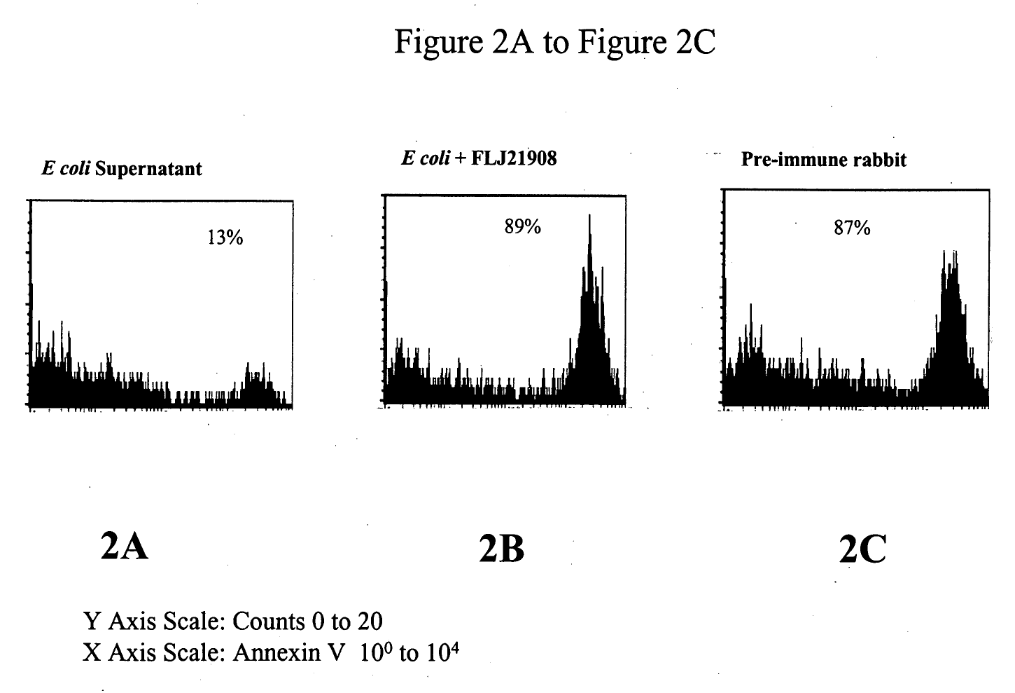

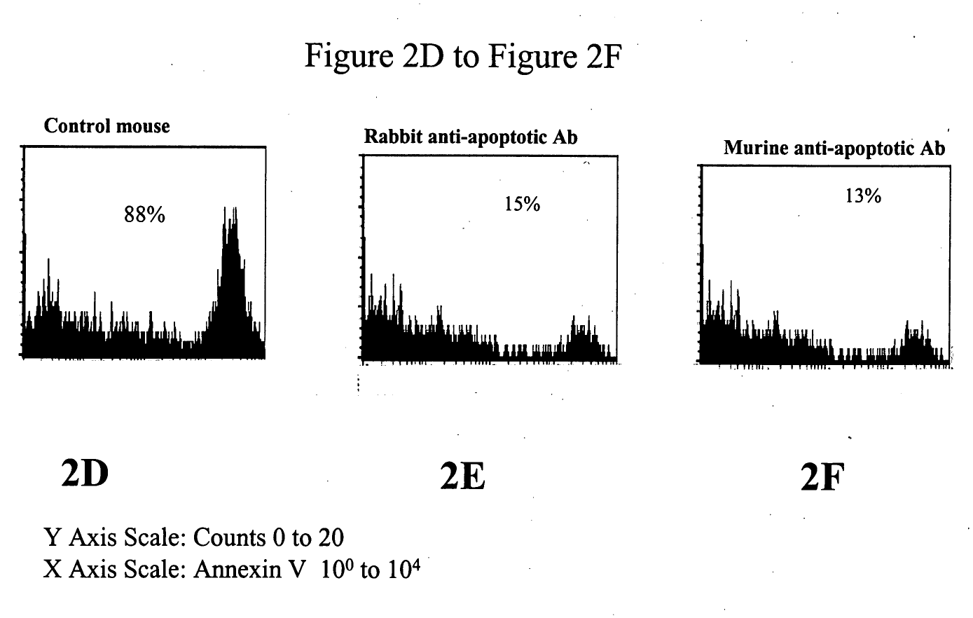

The 6000 d peptide was purified from the supernatant of E. coli expressing the SHIV A protein by lyophilizing and resuspending bacterial supernatant in 100 mM NaCl, 20 mM Tris HCl pH 7.5 and loading it onto a DEAE sepharose column. Fractions were eluted by increasing the salt concentration. The isolated fractions were western blotted with the rabbit anti-SHIV A antibodies. The western blot positive fractions were then silver stained to ascertain the purity of the protein separation. Fractions 39 to 49 that were eluted with 1 M NaCl demonstrated a band at 6000 d consistent with the previously described pro-apoptotic factor (FIG. 2).

### Example 3

### Biological Activity of SHIV A Protein

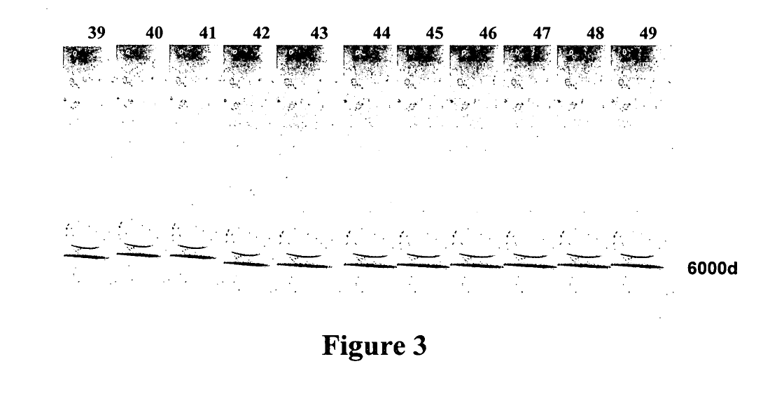

Since the SHIV A protein is identified herein as the proapoptotic factor, additional analyses of biological activity were conducted. Initially, Annexin V staining of unstimulated PBMC as target cells was used to determine the biological activity of the SHIV A protein. Supernatant from the SHIV A expressing E coli induced apoptosis in unstimulated target PBMC (FIG. 3).

To ensure that the SHIV A protein was a proapoptotic factor, polyclonal rabbit anti-proapoptotic factor and mouse anti-apoptotic monoclonal antibodies were used to block the apoptotic activity of the SHIV A protein (FIG. 3). The rabbit and murine anti-proapoptotic antibodies blocked SHIV A inducted apoptosis. Increased apoptosis was also observed when the SHIV A protein was added to PHA and anti-CD3 stimulated PBMC.

### Example 4

### Transfection of the FL14767485 Gene into BOSC Cells

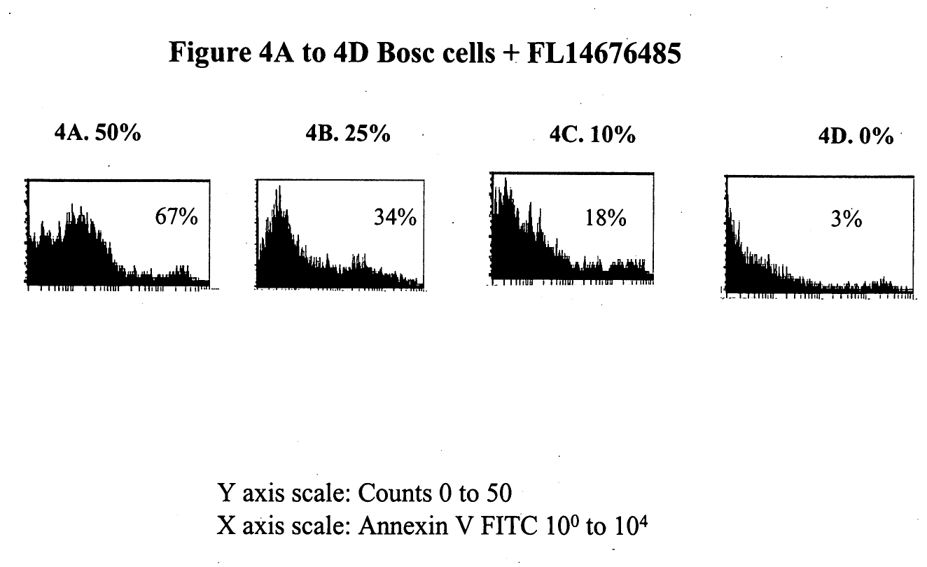

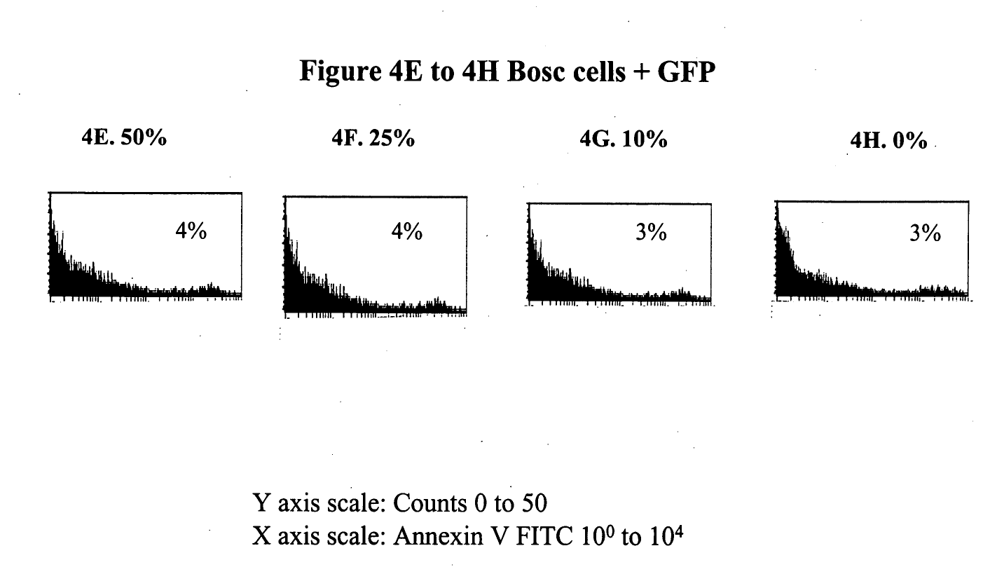

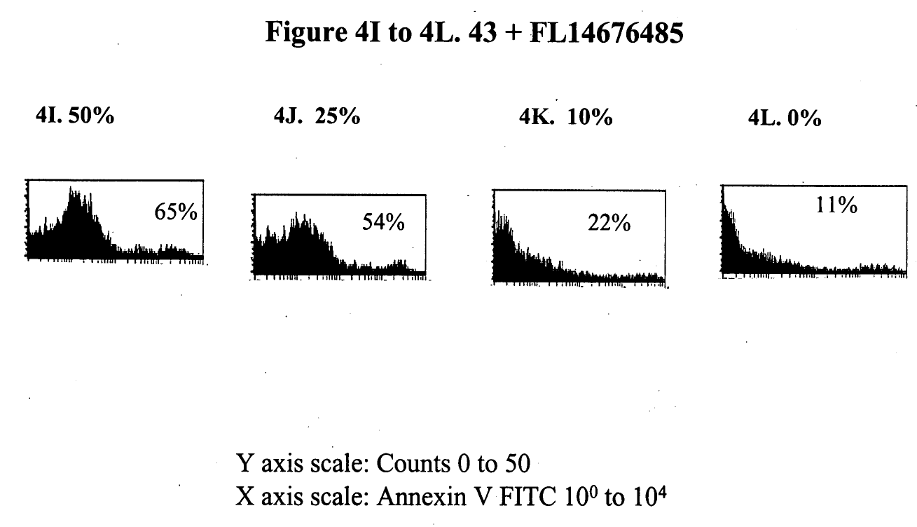

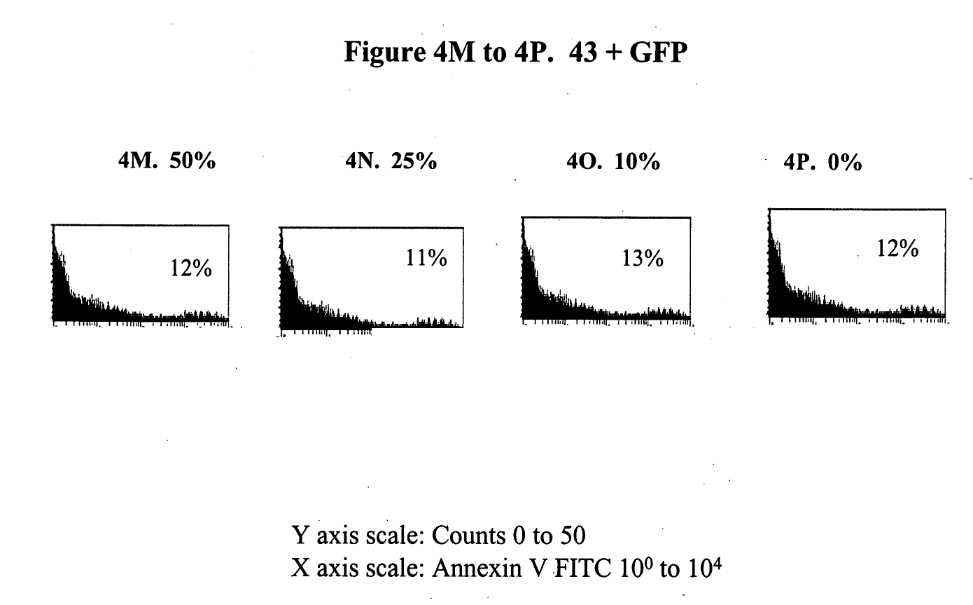

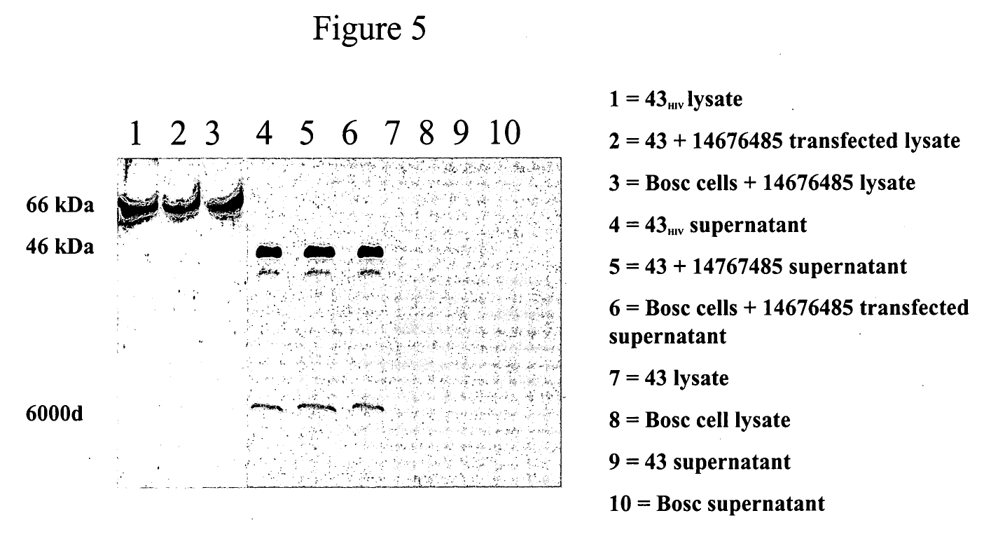

To further confirm the apoptotic activity of the SHIV A protein, the FL14676485 containing plasmid that encodes the SHIV A protein and green florescent protein (GFP to assess the efficiency of transfection into a human cell expression system), was transfected into Bosc cells, and into non-HIV-1 infected 43 cells. Bosc cells are derived from the 243T human embryo kidney cell line and are efficiently transfected with CaPO₄ (Pear et al., Proc Natl Acad Sci USA 90:8392, 1993). Mock transfection of the Bosc cells using GFP demonstrated that 90% of the cells were transfected. CaP0₄ also was used to transfect the 43 cells with the FL14767485 cDNA. In the 43 cells, the efficiency of the transfection using GFP was 30%. Transfection of the FL14767485 gene with DEAE dextran and Superfect produced similar rates of transfection in the 43 cells. After transfection, apoptotic activity was determined in the supernatant from Bosc cells and the 43 cells by Annexin V staining using unstimulated PBMC as target cells. A dose dependent increase in Annexin V staining was observed in PBMC incubated with supernatants from the Bosc and 43 cells transfected with FL14767485 gene but not in Bosc and 43 cells transfected with GFP (FIG. 4). Western blot analysis was performed using the supernatant and lysate of the untransfected and transfected 43 and Bosc cells along with 43_{HIV} cells using the polyclonal rabbit anti-pro-apoptotic factor antibodies to determine if the 6000 d peptide was being produced (FIG. 5). A protein with a molecular weight of 66 kDa corresponding to the SHIV A protein was detected in the lysate of the FL14676485 transfected 43 and Bosc cells and 43_{HIV} cells while a doublet of 46 kDa and a 6 kDa band were found in the supernatant. The 6000 d molecular weight protein corresponded to the pro-apoptotic factor. There was no detectable protein in either the lysate or supernatant of the untransfected 43 and Bosc cells (FIG. 5).

### Example 5

### Demonstration of FL14767485 RNA in 43_{HIV} Cells

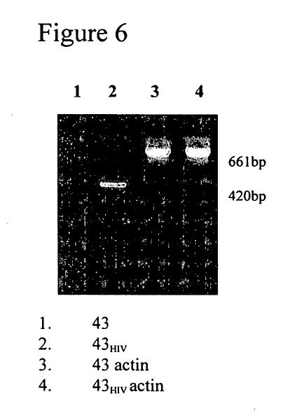

Studies were conducted to determine whether RNA for the SHIV A protein is constitutively expressed in 43 cells or whether it is induced after HIV-1 infection. PCR was used to determine the presence of RNA for the FL14767485 gene in the uninfected 43 cells and in 43_{HIV} cells. Forty-three cells were either left alone in culture or infected with either HIV-1_{IIIB} or HIV-1_{BaL} for 35 days and RNA was harvested for PCR analysis. Using HIV-1_{IIIB} base pair fragments consistent with the predicted size of the FL14767485 (420 bp) were observed in the 43_{HIV} cells but not in the non-HIV-1 infected 43 cells demonstrating that HIV-1 infection induces the SHIV A protein (FIG. 6). Actin (661 bp) was the positive control. Similar results were observed with 43 cells infected with HIV-1_{BaL}.

### Example 6

### Induction of Apoptosis by the SHIV A Protein

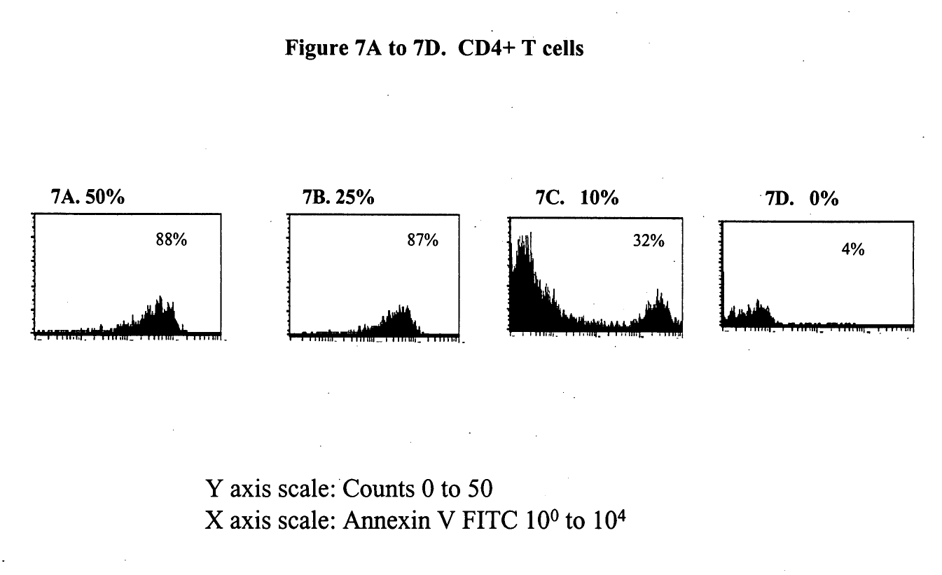

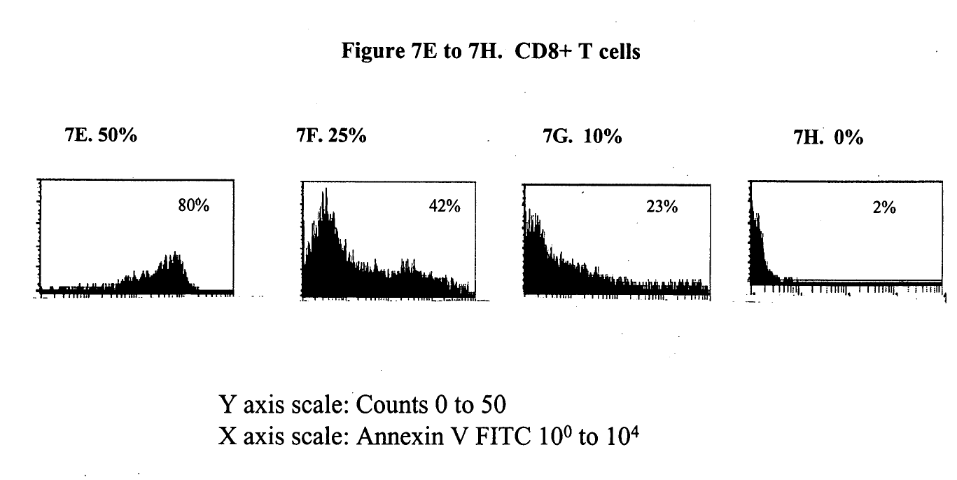

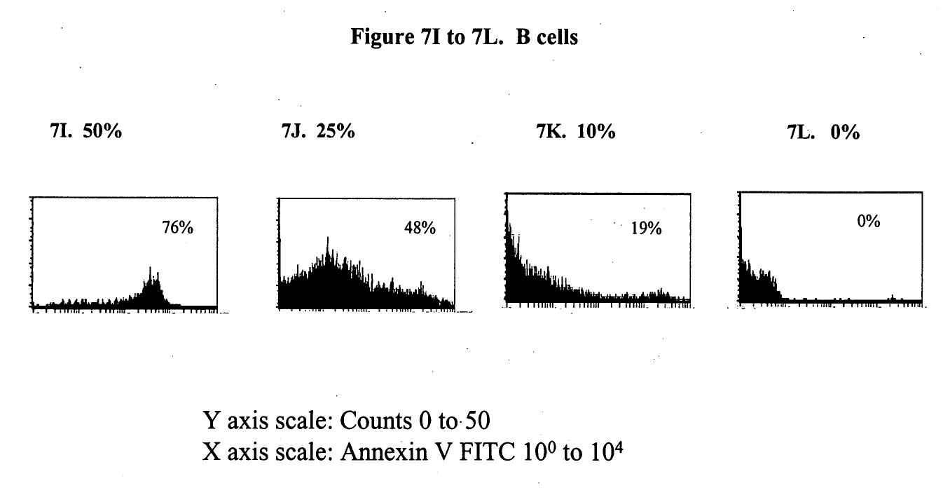

CD4+ and CD8+ cells and B cells: In the initial studies describing the pro-apoptotic factor, apoptotic activity was demonstrated in CD4+ and CD8+T cells as well as B cells (Chen et al., J Immunology 161:4257, 1998). The presence of the proapoptotic activity in the supernatant containing the SHIV A protein also was determined. Purified CD4+ and CD8+ T cells and B cells populations were isolated and purified and contacted with different concentrations of supernatant containing the SHIV A protein (50%, 25%, 10% and 0%) to demonstrate the induction of apoptosis. Similar to the 43_{HIV} derived pro-apoptotic factor, apoptotic activity from supernatants of the FL14676485 transfected Bosc cells containing the SHIV A protein was demonstrated for CD4+, CD8+ T cells and B cells by Annexin V staining (FIG. 7).

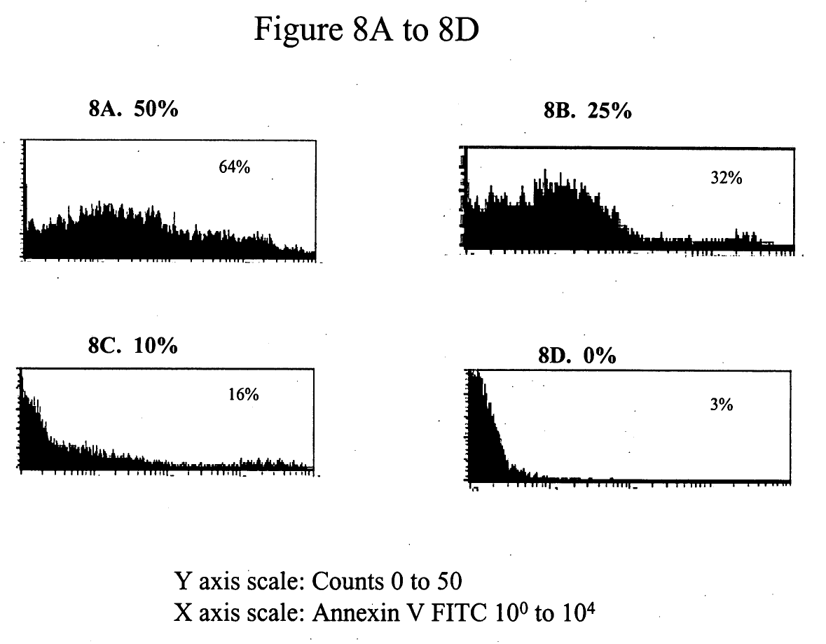

Demonstration of apoptotic activity in murine splenocytes: It is possible that the pro-apoptotic factor might be conserved in other animal species. If the SHIV A protein has pro-apoptotic activity in other species, it would be particularly useful in further studies to determine its biologic significance. To test this hypothesis, the effects of the proapoptotic factor on murine splenocytes was determined. Different concentrations of the SHIV A protein (50%, 25%, 10% and 0%) from the supernatants of Bosc cells were added to the murine splenocytes for 2 hours similar to the approach that was used with the human T cells and apoptosis was assessed by Annekin V staining. In line with the results obtained with the human T cells, supernatant containing the SHIV A protein induced apoptosis in the murine splenocytes (FIG. 8).

Role of the SHIV A protein in neuronal apoptosis: Apoptosis of neurons is a prominent feature of AIDS dementia (Kaul et al., Nature 410:988, 2001). Macrophages play an important role in this process. Macrophages and microglia cells infected with HIV-1 produce neurotoxins that damage neurons by releasing exictotoxins that produce excessive activation of glutamate receptors, primarily of the N-methyl-D-aspartate type (NMDAR) (Kaul et al., Nature 410:988, 2001). To determine a role for the SHIV A protein in neuronal apoptosis, the effect of SHIV A protein on the induction of apoptosis in the neuroblastoma cell line SH-SY5Y was investigated.

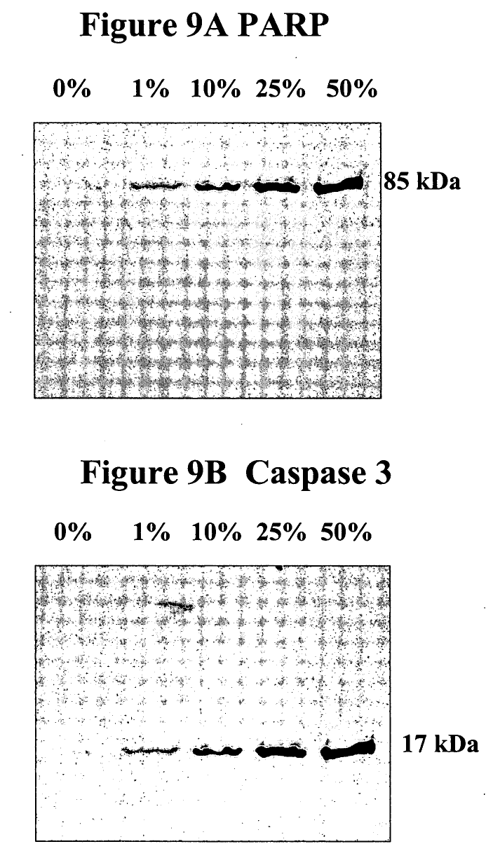

In these studies, different concentrations of the SHIV A protein (50%, 25%, 10%, and 0%) derived from the supernatants of Bosc cells were added to cultures of the neuronal cell line SH-SY5Y and apoptosis assessed by the induction of caspase-3 and poly (ADP-ribose) polymerase (PARP). Caspase-3 is a key protease that is activated during the early phases of apoptosis (Strasser et al., Annu Rev Biochem 69:217, 2000). Active caspase-3, a marker for cells undergoing apoptosis consists of a heterodimer of 17 and 12 kDa subunits that are derived from the 32 kDa pro-enzyme. Active caspase-3 proteolytically cleaves and activates other caspases as well as relevant targets in the cytoplasm e.g. D4-GDI and Bcl-2 and PARP in the nucleus. PARP is a 116 kDa nuclear chromatin-associated enzyme that catalyzes the transfer of ADP-ribose units from NAD+ to a variety of nuclear proteins including topoisomerases, histones, and PARP itself (Strasser et al., Annu Rev Biochem 69:217, 2000). During apoptosis PARP is cleaved from its 166 kDa intact form into 85 kDa and 25 kDa fragments. In the SH-SY5Y cultures incubated with supernatant from Bosc cells containing the SHIV A protein, the 17 kDa subunit of caspase-3 (Patel et al., FASEB J 10: 587, 1996) was demonstrated along with the 85 kDa fragment (D'Amores et al., Biochem J 342:249) of PARP (FIG. 9).

### Example 7

### Presence of the PRO-apoptotic Factor in Macrophages in HAD.

The presence of the SHIV A protein in histological sections from patients with HAD also was determined. In these studies, normal brain, Alzheimer's disease, and non-HIV-1 encephalitis were used as controls. Lymph tissue from the same patients also was used. These samples were obtained from Mount Sinai Medical Center, which is a part of a national NeuroAIDS consortium that provides well-characterized central nervous system (CNS) and peripheral nervous system (PNS) tissue samples and fluids from HIV-1 infected patients (Morgello et al., Neuropathology and Applied Neurobiology 27:326, 2001). Widespread reactive astrocytosis, myclin pallor, and infiltration predominantly by monocytoid cells, including blood-derived macrophages, resident microglia and multinucleated giant cells, characterize the neuropathology associated with HIV infection of the brain (Lipson and Gendelman, N Eng J Med 332:934, 1995).

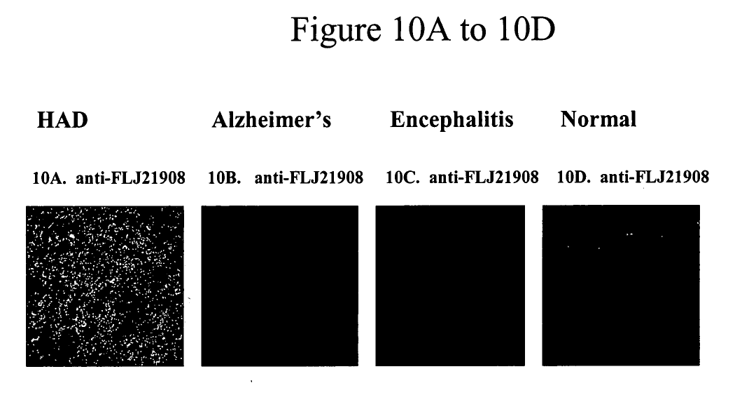

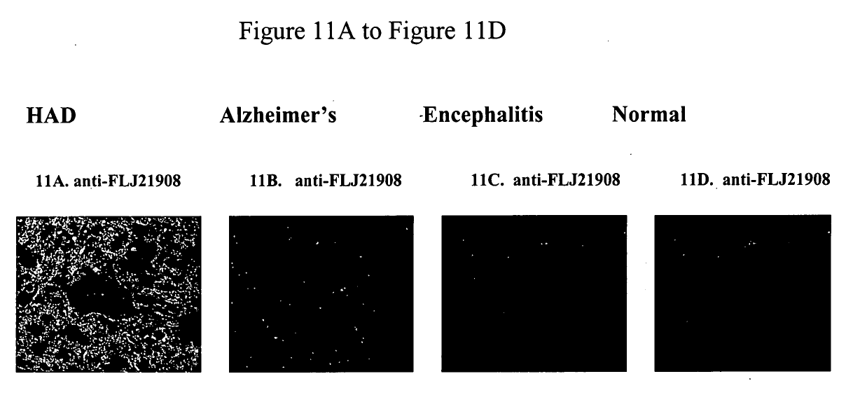

Neurological apoptosis is not specific for HAD but is a feature of many different types of dementia caused by infectious agents and other neurodegenerative diseases including Alzheimer's disease (Kaul et al., Nature 410:988, 2001). Using the murine anti-SHIV A antibody, punctate green staining consistent with the presence of the SHIV A protein was detected in patients with HAD but not in normal, Alzheimer's disease or non-HIV-1 encephalitis patients (FIG. 10). Lymph nodes from the same patients also were stained for the presence of the SHIV A protein that were used to study brain tissue. Similar to the results obtained with the brain tissue, the SHIV A protein was present in lymph nodes from the HIV-1 infected patients (FIG. 11).

### Example8

### Further Characterization of SHIV A

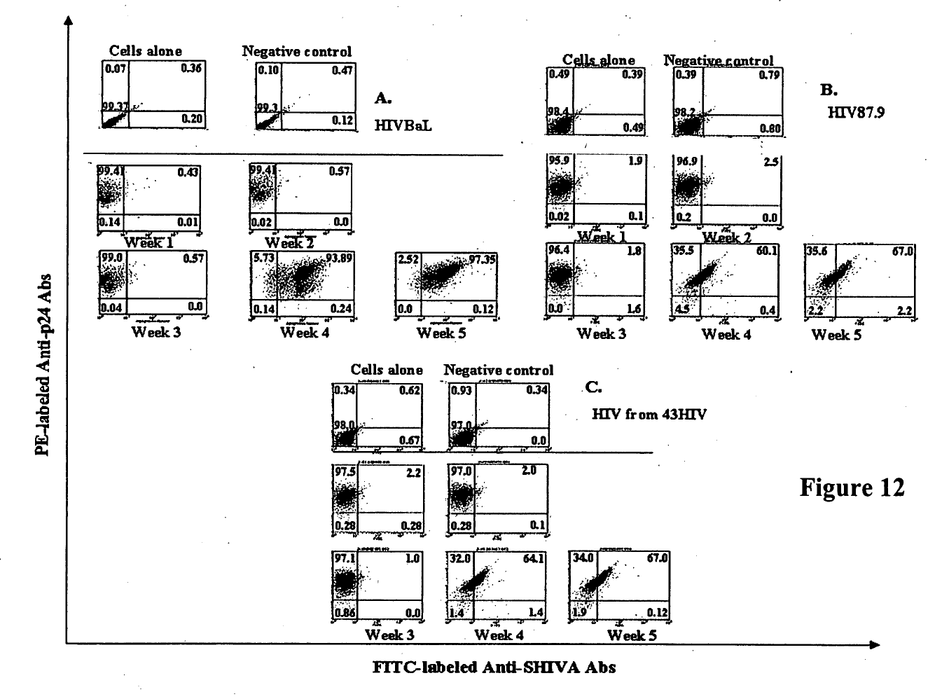

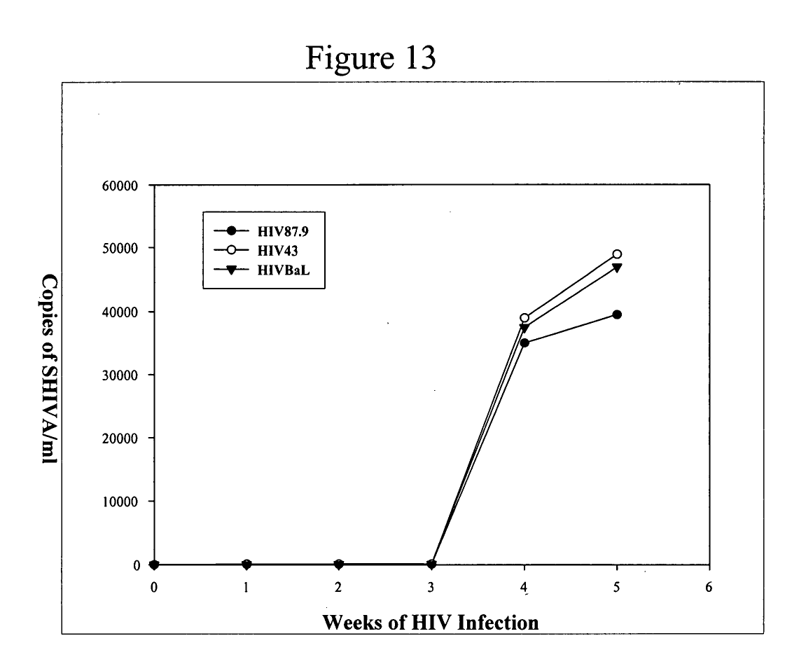

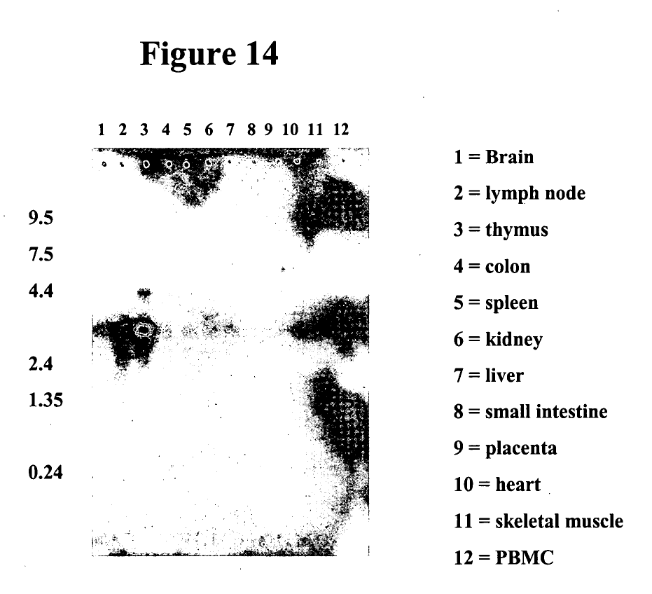

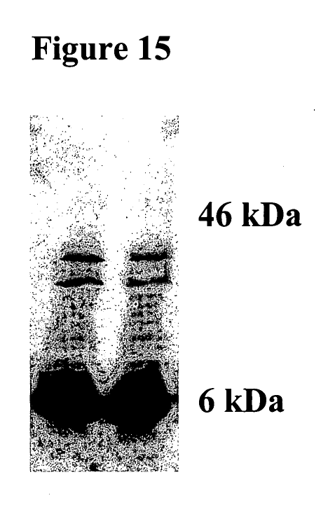

This Example provides a more detailed characterization of the protein described in Examples 1-7. The production of SHIV A by the 43_{HIV} cells as determined by intracytoplasmic staining occurred 4 weeks after HIV-1 infection (FIG. 12A) and was associated with the appearance of CCR5 and CXCR4 co-receptor usage by progeny viruses (Table I). However, dual tropic HIV-1 isolates along with HIV-1 produced by 43_{HIV} 5 weeks after infection, did not induce SHIV A production more efficiently than then pure CCR5 using HIV-1 isolates (FIGS. 12A, 12B and 12C) so viral tropism is not related to SHIV A production. It was demonstrated by real time PCR that mRNA for SHIV A was induced 4 weeks after HIV-1 infection (FIG. 13). The presence of the SHIV A gene was probed in different tissue types by northern blot analysis and detected it only in the thymus and in the lymph nodes (FIG. 14). A SHIV A fusion protein that has apoptotic activity was generated (FIG. 15).

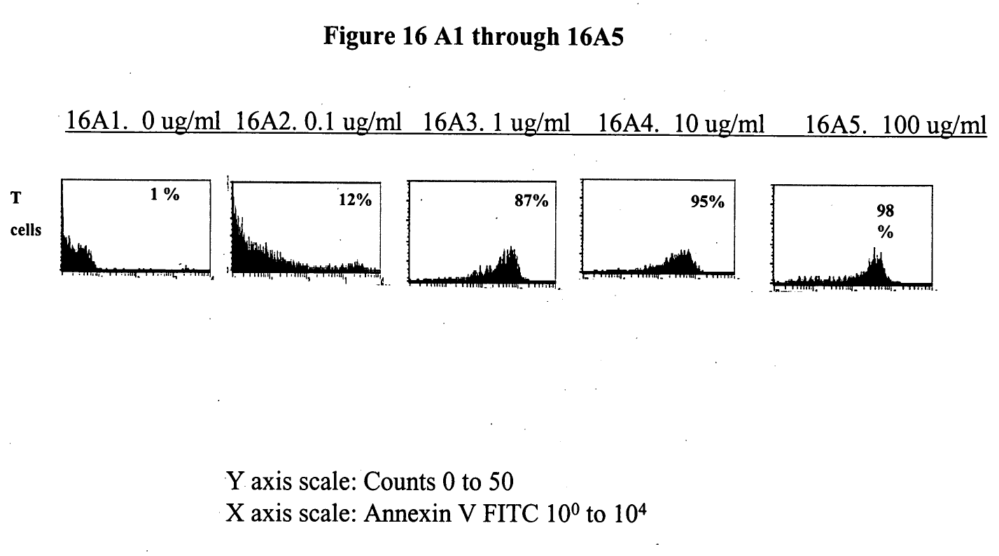

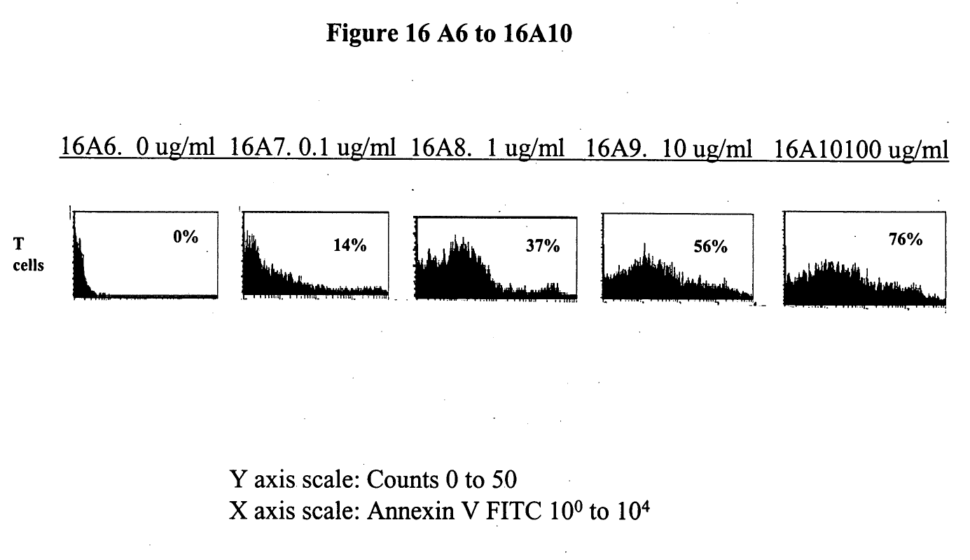

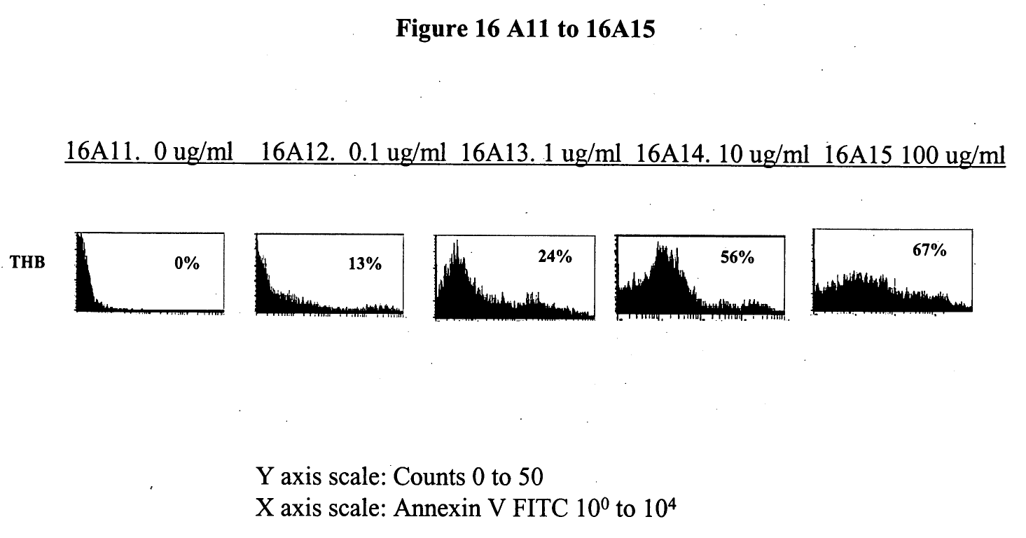

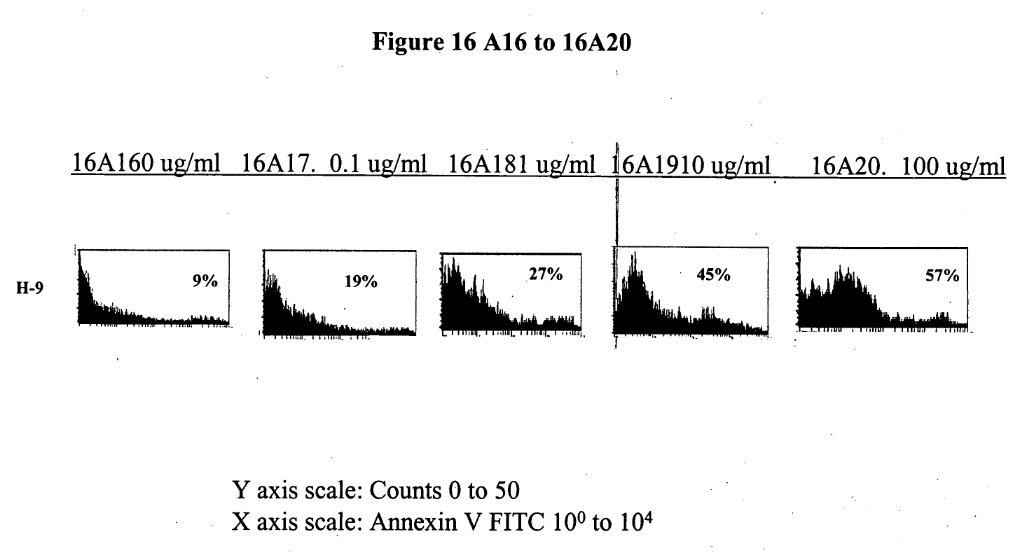

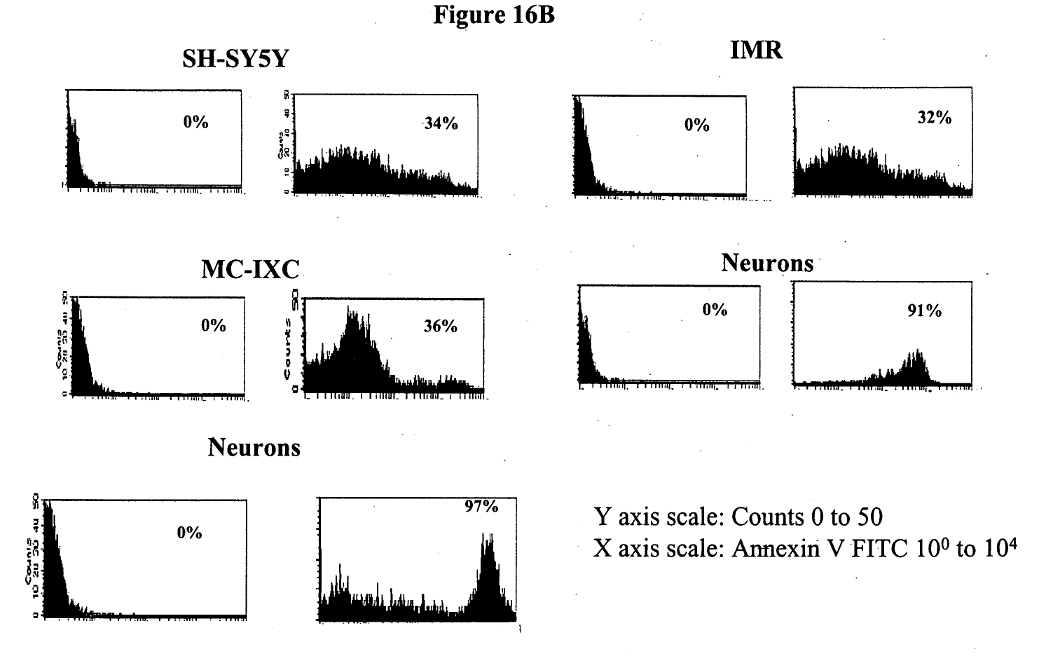

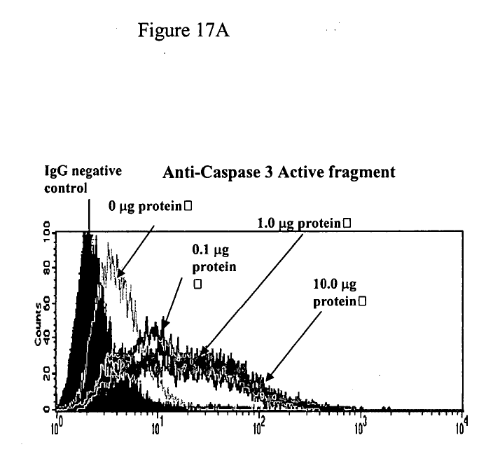

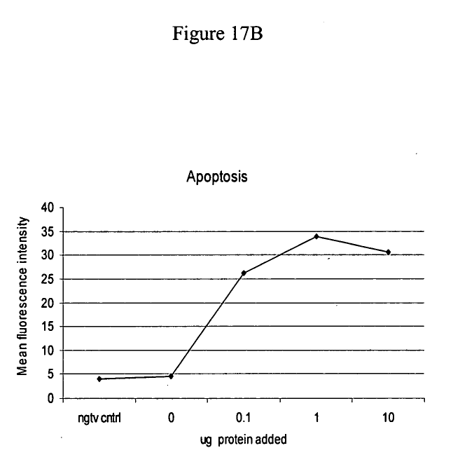

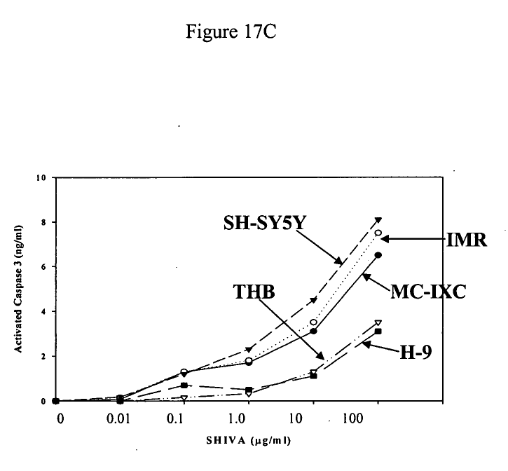

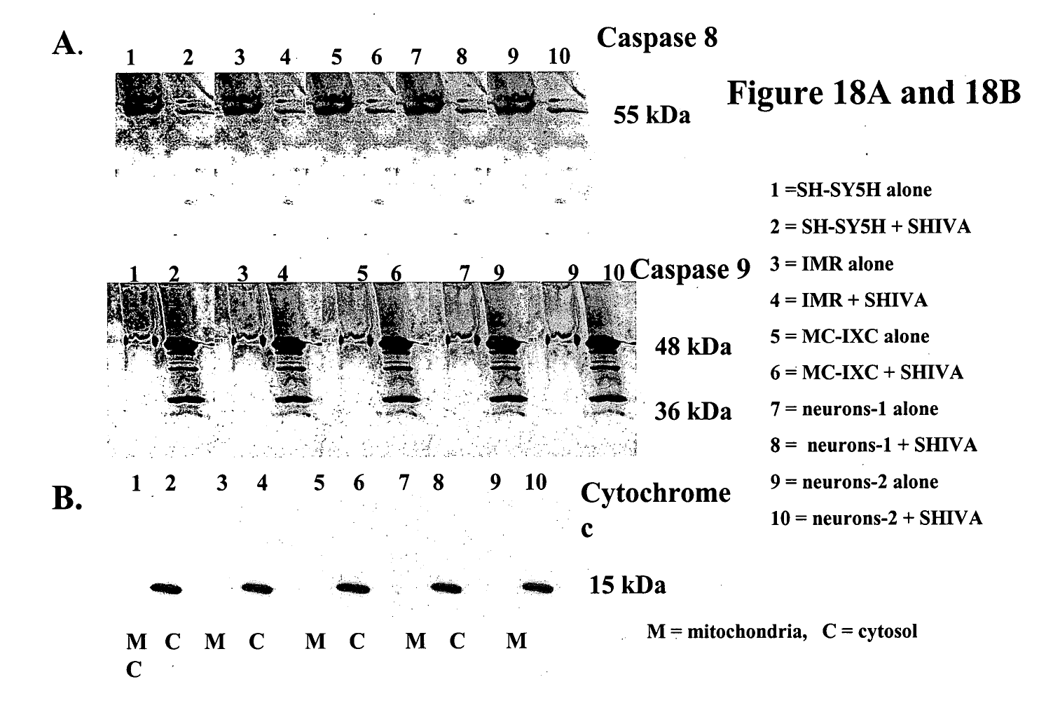

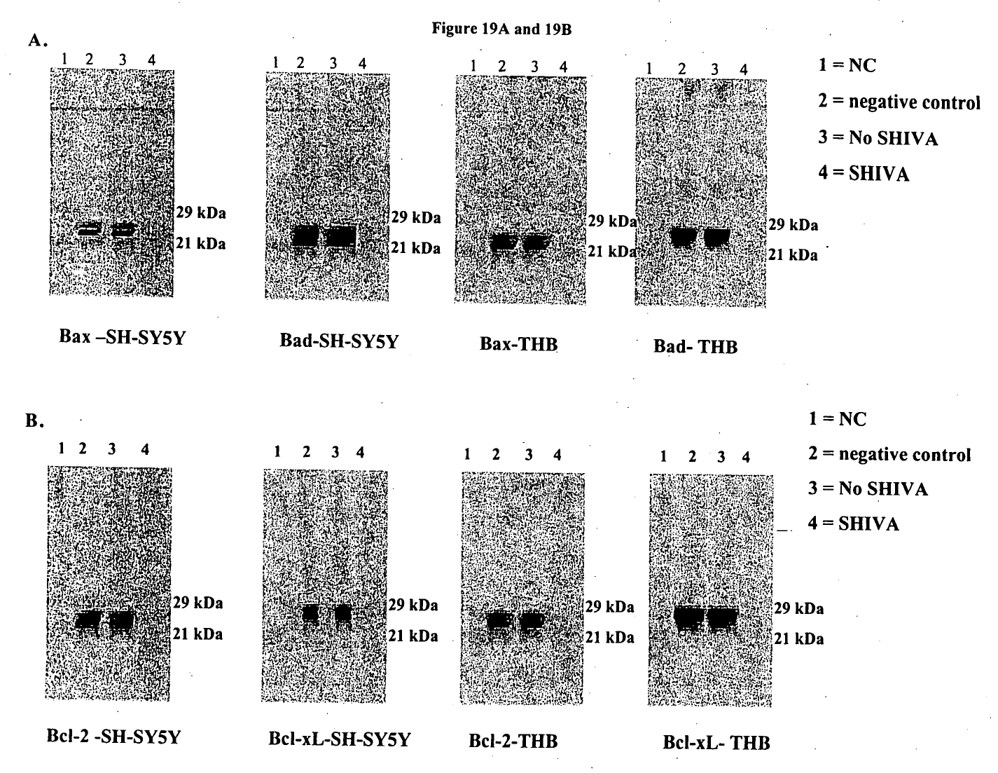

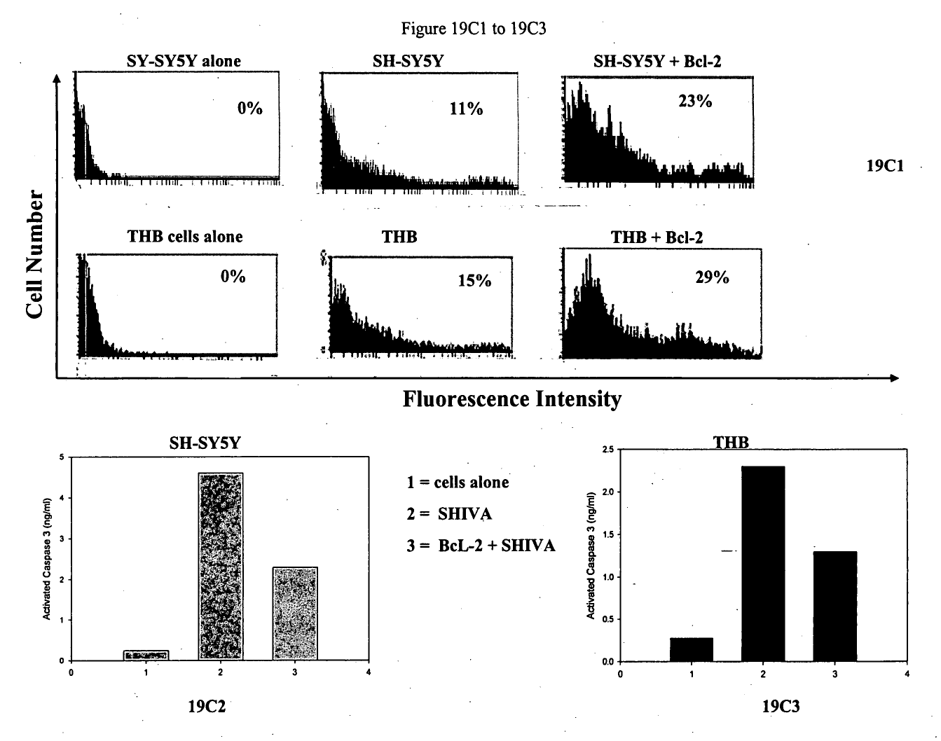

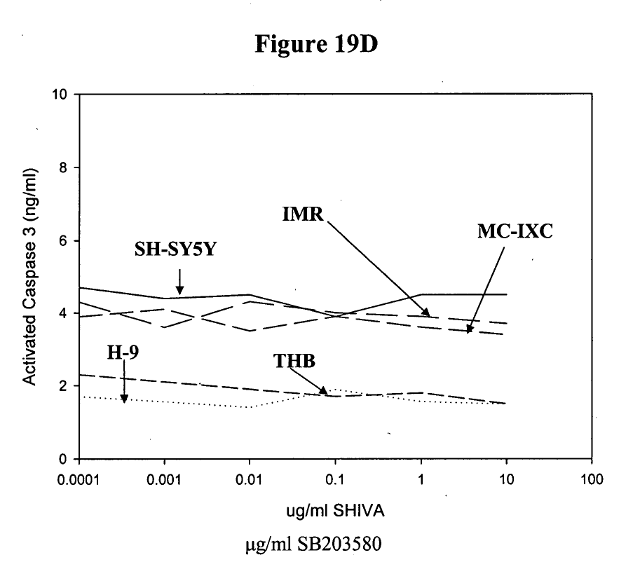

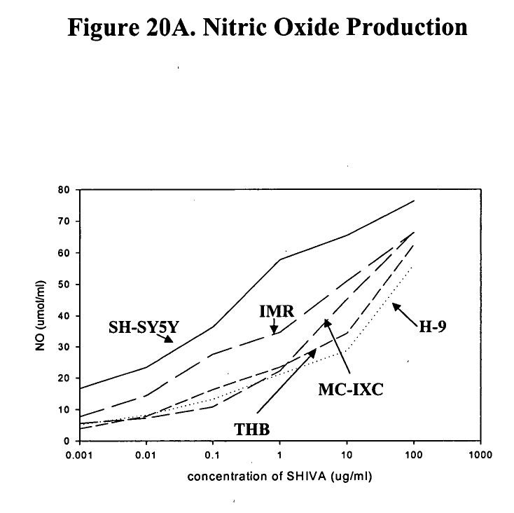

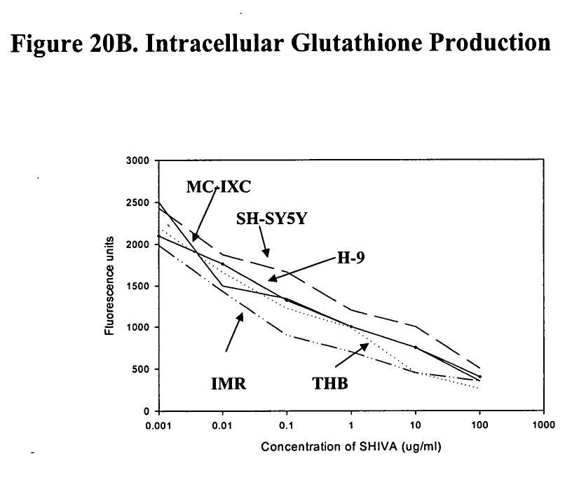

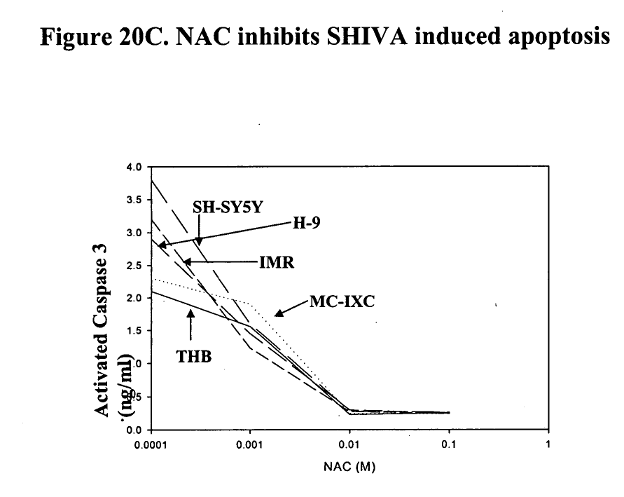

Using the fusion protein generated herein, it was shown that that SHIV A is more potent in inducing apoptosis in neurons than it is in T cells and B cells (FIGS. 16, 17A, 17B, and 17C) and that it does not appear to induce apoptosis in astrocytes and epithelial cells (respiratory, gastrointestinal and uterine). The SHIV A fusion protein induced apoptosis by fluxing calcium, releasing cytochrome c from the mitochondria, activating Caspase 9 (FIGS. 18A and 18B) that is associated with the activation of Bad and Bax and suppression of Bcl-2 and Bcl-xL (FIGS. 19A and 19B). Furthermore, there is no activation of the MAP kinase pathway (FIG. 19C). SHIV A also results in the induction of nitric oxide production (FIG. 20A), loss of intracellular glutathione (FIG. 20B) and can be inhibited by memantidine (FIG. 20A), (anti-oxidants FIG. 20B) and transfection with Bcl-2 (FIGS. 20C and 20D).

Presence of the SHIV A protein in the 43_{HIV} cells at different points after infection: To more clearly focus on the natural history of SHIV A expression by the 43_{HIV} cell line, the inventors studied the production of SHIV A as it relates to co-receptor usage by HIV-1 produced during the course of infection. Increased apoptosis and HAD occur late in the course of HIV infection where there are both CXCR4 and CCR5 co-receptor using HIV present, making it possible that CXCR4 co-receptor using virions could induce SHIV A. 43 cells were infected with HIV-1_{BaL} (FIG. 12A), a monocytotropic strain of HIV-1 and determined infection and production of SHIV A by dual intracytoplasmic staining using PE-labeled anti-p24 mAbs and FITC-labeled anti-SHIV A monoclonal antibodies. The co-receptor usage of the progeny virions was also followed at weekly intervals by infecting target cells that expressed either CCR5 (U87.CD4.CCR5) or CXCR4 (U87.CD4.CXCR4).

As illustrated in FIG. 12A, the 43 cells were uniformly infected with HIV-1_{BaL} after 1 week of infection but the production of SHIV A did not occur until the fourth week. The production of SHIV A by the 43_{HIV} cells coincided with the appearance of dual tropic HIV isolates at weeks 4 and 5 (Table I).

TABLE I
Co-receptor usage by the HIV-1 produced by 43_{HIV} cells during the
course of infection

| Week | U87.CD4.CCR5 | U87.CD4.CXC4 | |
|---|---|---|---|
| 1 | 1233 pg/ml | 0 | |
| 2 | 1500 pg/ml | 0 | |
| 3 | 1322 pg/ml | 0 | |
| 4 | 1433 pg/ml | 1110 | pg/ml |
| 5 | 1222 pg/ml | 1211 | pg/ml |

In order to generate the data shown in Table I, U87.CD4.CCR5 and U.87.CD4.CXCR5 cells were obtained from the AIDS Research, Reference and Reagent Program, cultured in DMEM, 15% FCS, supplemented with μg/ml puromycin, 300 μg/ml G418, glutamine, and 1% pen/strep, and then infected with HIV-1 produced by 43_{HIV} at weekly intervals after infection with HIV-1_{BaL}. The HIV-1 used to infect the U.87.CD4.CCR5 and U87.CD4.CXCR4 cells was standardized to contain equivalent amounts of virus based on reverse transcriptase activity (80,000 cpm/ml). HIV-1 infection was determined by measuring the presence of p24 in the culture supernatant by ELISA 7 days after of infection.

Before infection of the 43 cells the HIV-1_{BaL} was used to infect the CCR5 (U87.CD4.CCR5) or CXCR4 (U87.CD4.CXCR4) target cells to ensure that there were no CXCR4 using quasi-species in the viral inoculate since it was previously reported that the 43 cells could be infected with CXCR4 co-receptor using viral strains including HIV-1_{IIIB} and HIV-1_{89.6}. Active HIV replication was not in the CXCR4 (U87.CD4.CXCR4) target cells when infected with different MOIs of HIV-1_{BaL}, thereby eliminating the possibility that there were CXCR4 utilizing strains over the 5 weeks of infection that resulted in multiple re-infection cycles and not a switch to dual tropic viruses. In other experiments, the p94UG114.1 infectious clone that contains a pure CCR5 co-receptor was transfected using HIV-1 virus into Bosc cells to obtain HIV-1 to infect the 43 cells. Again, it was noted that the production of SHIV A by the 43_{HIV} cells infected with HIV-1 derived from the p94UG114.1 clone coincided with the appearance of dual tropic HIV-1 isolates at weeks 4 and 5.

There was no expression of active Caspase 3 in the 43_{HIV} cells making outgrowth of a survivor population less likely. SHIV A failed to induce apoptosis in uninfected 43 cells and UV treated supernatant from 43_{HIV} cells infected for 1, 2, 3, 4 and 5 weeks did not induce SHIV A in the uninfected 43 cells. Since the production of SHIV A coincided with the appearance of dual tropic viruses, the inventors next investigated if dual tropic viruses could induce SHIV A production more rapidly than HIV-1_{BaL}. To test this, 43 cells were infected with a dual tropic virus, HIV-1_{87.9} and HIV-1 produced by the 43_{HIV} cells 5 weeks after infection. Similar to the results that we observed with HIV-1_{BaL}, the production of SHIV A occurred 4 and 5 weeks after infection (FIGS. 12B and 12C).

Regulation of SHIV A mRNA production: As demonstrated in FIGS. 12A, 12B, and 12C, SHIV A production occurred in the 43 cells 4 weeks after HIV-1 infection with either monocytotropic or dual tropic viruses. It is possible that SHIV A mRNA was induced early after infection, but was not translated or alternatively that mRNA transcription does not occur until after 3 weeks of infection. To address this, the inventors determined at what time point after HIV-1 infection SHIV A mRNA was induced in the 43 cells after HIV infection using real time PCR. In these experiments, mRNA was isolated from the 43_{HIV} cells at different points (1, 2, 3, 4 and 5 weeks) after infection with HIV_{BaL}, HIV_{87.9}, and HIV from 43_{HIV} cells and real time PCR performed. As is illustrated in FIG. 13, there were background levels of mRNA for SHIV A (50 copies) detected in the 43_{HIV} cells 3 weeks after infection with HIV_{BaL}, HIV_{87.9} and HIV from 43_{HIV} cells 5 weeks that were rapidly increased to 38,000 and 47,000 copies in HIV_{BaL}, to 32,000 and 37000 copies in HIV_{87.9}, and to 39,500 and 51,000 copies for HIV from 43_{HIV} cells after 4 and 5 weeks 15 of infection (FIG. 13).

Detection of SHIV A in different tissues: Although SHIV A was identified as being produced by HIV infected macrophages, it may have been possible that other cell types have the capacity to produce this protein and in fact it may be a normal constituent of cell growth and regulation. The availability of specific probes allowed the inventors to determine if other tissues express SHIV A. Multiple human tissues were probed by northern blots including heart, brain, placenta, lung, liver, skeletal muscle, kidney, pancreas, testes, ovary, small intestine, colon, peripheral blood leukocytes, lymph nodes, bone marrow, fetal liver and thymus and detected the presence of SHIV A (2.8 kB) only in thymus and lymph node, sites where apoptosis occurs (FIG. 14).

Generation of a SHIV A fusion protein: To better define the apoptotic capacity of the SHIV A protein, a GST SHIV A fusion protein was generated. Initially 2 fusion proteins were made, one containing the first 330 amino acids and the second one containing the last 330 amino acids of SHIV A. Both fusion proteins were tested for apoptotic activity using the THB T cell line and the SY-SYSY neuronal cell line as target cells. Only the fusion protein from amino acids 330 to 660 had apoptotic activity. The purity of the apoptotic fusion protein was assessed by electrophoresis on a 15% SDS-polyacrylamide gel followed by Coomassie Blue staining. A band with a molecular weight of 33 kDa was detected corresponding to the fusion protein along with a 6000 d peptide corresponding to the originally described pro-apoptotic peptide (Chen et al., J Immunol 1998; 161:4257) (FIG. 15).

Spectrum of SHIV A apoptotic activity: Using the SHIV A fusion protein, the spectrum of apoptotic activity was determined by determining if other cell types underwent apoptosis after exposure to SHIV A. The inventors tried to induce apoptosis in respiratory (A549, BEAS-2B), gastrointestinal (HT-29, T84, Caco-2), fibroblast (IRR-MRC-5) and uterine epithelial (Ishikowa) cell lines along with a fibroblast cell line (IRR-MRC-5) but failed to induce apoptosis at any concentration (0.01, 0.1, 1, 10, and 100 μg/ml) of the SHIV A fusion protein that was used. The inventors also tried to induce apoptosis using different concentrations of the SHIV A fusion protein in astrocytoma cell lines (CCF-STTG1, SW1088) but again were unsuccessful. The only cell lines that underwent apoptosis in response to SHIV A were T cell lines (THB, H-9), primary T cells (FIG. 16), B cell lines (Laz, Ramos) and primary B cells and 3 neuroblastoma cell lines and 2 preparations of primary neurons (FIGS. 17b and 17C).

For the neuronal cell lines, SH-SY5Y, IMR, and the MC-IXC cell lines and 2 preparations of primary fetal neurons (primary neurons-1 and neurons-2) were used. There was Annexin V staining in the neuronal cells as determined by flow cytometry after treatment with SHIV A (FIG. 17A). Apoptosis in primary neurons also was assessed by intracytoplasmically staining for activated Caspase-3 (FIG. 17B). Interestingly, primary neuronal cell lines and primary neurons underwent apoptosis at a SHIV A fusion protein concentration of 1 μg/ml compared to the primary T cells and T cell lines where maximal apoptosis occurred after treatment with 100 μg/ml of SHIV A as determined by flow cytometry. To better quantify the apoptotic effect, levels of activated Caspase-3 were measured by ELISA in SHIV A treated (0.01, 0.1, 1, 10, and 100 μg/ml) T cell lines and neuronal cell lines and again found that SHIV A was more potent in inducing apoptosis in neuronal cells than T cells (FIG. 17C).

Caspase 9 pathway of apoptosis: Examples 1-7 demonstrated that SHIV A induced apoptosis in the THB T cell line through the activation of Caspase 9 (Sperber et al., J Immunol 2003; 170:1566). These studies were extended to the SH-SY5H, IMR, and MC-IXC neuronal cell lines and primary neurons treating them with 1 μg/ml of the SHIV A fusion protein for 16 hours and performing western blot analysis for activated Caspase 8 and Caspase 9. Similar to the results that were obtained for the THB cells, breakdown fragments (30 kDa) for Caspase 9 but not Caspase 8 were detected by western blot in lysates from all 3 of the neuronal cell lines and in the 2 preparations of primary neurons (FIG. 18A). To further confirm that Caspase 9 was activated in the induction of apoptosis by SHIV A, primary T cells, THB, H-9 cells, primary neurons, and the SH-SY5Y neuroblastoma lines were treated with a Caspase 9 inhibitor prior to treatment with SHIV A and blocked apoptosis. Caspase 9 activation occurs through a mitochondrial pathway of apoptosis and is associated with the release of cytochrome c from the mitochondria into the cytoplasm (Kuida et al., Cell 1998; 94:325, Ashkenazi, Science 1998.281:1305). In order to test whether this was occurring during the induction of apoptosis mediated by SHIV A in neuronal tissue, mitochondrial and cytosolic fractions were isolated from the neuronal cell lines and primary neurons and performed western blot analysis for cytochrome c. In the absence of treatment with the SHIV A fusion protein, there was no detectable cytochrome c in the cytoplasm of the neuronal cell lines and primary neurons. However, after treatment with 1 μg/ml of the SHIV A fusion protein, cytochrome c (15 kDa) was present in the cytosolic fractions of the neuronal cell lines and primary neurons consistent with a mitochondria pathway of apoptosis induction (FIG. 18B). The inventors also investigated whether SHIV A induced calcium flux in the panel of target cell lines. The SH-SY5Y, IMR, MC-IXC, THB, and H-9 cell lines were stimulated with 100 μg/ml of SHIV A to determine if there was calcium flux. SHIV A induced a 3-fold increase in calcium flux in the SH-SY5H, IMR, and MC-IXC neuronal cells and a 2-fold increase in the THB and H-9 cells.

Mitochondrial pathway of SHIV A induced apoptosis: Since SHIV A utilizes a Caspase 9 mitochondrial pathway of apoptosis, the inventors investigated if other pro and anti-apoptotic mitochondrial proteins were activated. SH-SY5Y and THB cells were treated with SHIV A for 16 hours and performed western blot analysis using a panel of antibodies directed used in mitochondrial apoptosis including Apaf-1, Bad, Bax, Bcl-2, Bcl-xL, Bruce, CAS, hILP/XIAP, Mcl-1, Nip1 and p53 protein. These antibodies recognize non-activated proteins. There was no activation of Apaf-1, Bruce, CAS, hILP/XIAP, Mcl-1, Nip1 and the p53 protein in the SH-SY5Y and THB cell lines. However, as demonstrated in FIGS. 19A and 19B, there was activation (negative western blot) of Bad, Bax, Bcl-2 and Bcl-xL in the SHIV A treated cells but not in the untreated cells). The inventors further attempted to block SHIV A induced apoptosis by transfecting full-length Bcl-2 driven by a CMV promoter into SH-SY5Y and THB cells. The efficiency of the transfection was 25% and 27% respectively for the SH-SY5Y and THB cells and it was possible to increase Bcl-2 expression from baseline levels of 11% and 15% to 23% and 29% respectively (FIG. 19C). When both the SH-SY5Y and THB cells were treated with 100 μg/ml of SHIV A, apoptosis was reduced by 50% (FIG. 19). The inventors also investigated if there was SHIV A-induced MAP kinase activation in the SH-SY5Y, IXR, MC-IXC, THB, and H-9 cells. They attempted to block SHIV A induced apoptosis with the MAP kinase inhibitor, SB203580. As is illustrated in FIG. 19C, SB203580 at every concentration tested had no effect on SHIV A induced apoptosis.

NO and glutathione production: The effects of SHIV A in the induction of NO production or affects intracellular glutathione levels in the panel of target cells. SH-SY5Y, IMR, MC-IXC, THB and H-9 cells were stimulated with SHIV A and measured nitrite production as a marker for NO production. As noted in FIG. 9A, there was a dose dependent increase in nitrite production after SHIV A treatment in all of the cell lines tested. The inventors next determined if SHIV A alters intracellular levels of glutathione in our panel of target of test cells. After treatment with SHIV A, there was a dose dependent decrease in intracellular glutathione levels in the SH-SY5Y, IMR, MC-IMR, MC-IXC, THB and H-9 cells (FIG. 20B).

Blocking SHIV A protein induced apoptosis with anti-oxidants: As noted in FIG. 20B, SHIV A causes a decrease in intracellular glutathione levels causing oxidative stress. The inventors further attempted SHIV A induced apoptosis by treating cells with an anti-oxidant, N-acetyl cysteine (NAC). The panel of target was treated with different concentrations of NAC and apoptosis was assessed. NAC blocked apoptosis. Since SHIV A acts through a Caspase 9 pathway we wanted to determine if SHIV A could be blocked by the NMDA antagonist memantine. In order to test this, the SH-SY5Y, IMR and MC-IXC, THB, and H-9 cells that undergo apoptosis in response to SHIV A were incubated with different concentrations of memantine (10⁻⁵ to 10⁻⁹ M) and then treated with 100 μg of the SHIV A fusion protein. Apoptosis was determined using the Caspase 3 ELISA.

Discussion: In FIG. 13, it can be seen that mRNA for SHIV A as determined by real time RNA PCR was at background levels (less than 100 copies) in the 43_{HIV} cells during the first 3 weeks of HIV-1 infection with HIV_{Bal.}, HIV_{89.6} and HIV from 43_{HIV} cells that increased markedly after four and 5 weeks of infection. The induction of SHIV A mRNA in the 43 cells after 3 weeks of HIV infection is an interesting aspect of SHIV A induction. This may be due to either increased mRNA initiation or alternatively by increased mRNA stability. To assess this more fully, transcriptional initiation experiments may be performed based on nuclear runoff experiments to determine SHIV A mRNA initiation whereas SHIV A RNA stability may be assessed by Northern blot analysis for SHIV A following Actinomycin D treatment. There could also be a post-transcriptional block caused by lack of nuclear to cytoplasm transport of mRNA for SHIV A, alternate splicing, or translational problems that could delay the appearance of the SHIV A protein until 4 weeks post infection.

Excitotoxins that cause excessive activation of NMDARs may be another mechanism whereby the SHIV A protein induces apoptosis in neuronal cells. SHIV A induces Ca²⁺ flux, releases of cytochrome c into the cytoplasm and activates Caspase 9 in the neuroblastoma cells, primary neurons, T cell lines and primary T cells similar to other excitotoxins including EEA, PAF and NO. It had been demonstrated that NMDAR antagonists prevent neuronal cell death in vitro resulting from HIV-1 infected macrophages or purified gp120. Transgenic mice expressing gp120 in the brain have neuropathologic changes similar to HAD that can be prevented by NMDAR antagonists. There are two pathways that over stimulation of the NMDAR receptors by neurotoxins induce apoptosis, a mitochondrial pathway and non-mitochondrial pathway. NMDAR stimulation leads to excessive Ca²⁺ influx into neurons. In the mitochondrial pathway the increased intracellular Ca²⁺ leads to loss of integrity of the mitochondrial inner member that leads to release of cytochrome c, free-radical NO, and reactive oxygen species (ROS), caspase activation and apoptosis. NMDARs are physically tethered to neuronal nitric oxide synthetase, facilitating its activation. In the non-mitochondrial pathways excessive Ca²⁺ triggers the activation of p38 MAP kinase that can lead to phosphorylation and activation of transcription factors involved in apoptosis. Interestingly, the NMDA receptor antagonist menantine can block SHIV A induced apoptosis that also causes NO release. There does not appear to be a non-mitochondrial pathway in SHIV A induced apoptosis since there was no activation of MAP kinase activity in SHIV A treated neuronal and T cell lines as determined by western blot using anti-p38 MAP kinase antibodies (normalized to total p38 levels by direct immunoblotting, FIG. 19).

In FIG. 14, the presence of SHIV A was detected in the thymus and lymph nodes in tissue blots where apoptosis commonly occurs. Since SHIV A was not only present in HIV infected monocytes, it could have some role in maintaining immune homeostasis through induction of apoptosis. The availability of the SHIV A fusion protein (FIG. 15) allowed the definition of the spectrum of this protein's apoptotic activity. Interestingly, bacterial proteases were capable of cleaving the fusion protein into the 6000 d apoptotic peptide (FIG. 15). This finding along with the results in FIGS. 12A, 12B, and 12C and 13, where protein production and mRNA for SHIV A are detected after 3 weeks of infection demonstrate that transcription of SHIV A mRNA results in the immediate production of the 6000 d peptide, making the effect of HIV protease on SHIV A production less likely. These results are consistent with the initial characterization of SHIV A discussed in Examples 1-7 above.

Functionally, SHIV A is more potent in inducing apoptosis in neuronal tissue (FIGS. 17A, 17B, 17C and 17D) than in T cells (FIG. 16). Other cells types inducing gastrointestinal, respiratory, and uterine epithelial cell lines along with fibroblasts, astrocytoma, and microglial cell lines did not undergo apoptosis in response to SHIV A. These findings strongly suggest that the activity of SHIV A is receptor mediated. The above discussed data show calcium flux, release of cytochrome c from the mitochondria into the cytoplasm and activation of Caspase 9 as the pathway that SHIV A uses to induce apoptosis in T cells and neuronal cells (FIGS. 18A, 18B, 18C). The pathway of apoptosis induction by SHIV A is similar to exticotoxins, glutamate-like substances that stimulate the NMDA receptor that have been implicated in HAD.

Apoptotic neurons do not co-localize with infected microglia in HAD patients, supporting the hypothesis that HIV infection causes neurodegeneration through the release of soluble factors of which SHIV A may be one. Systems designed to study the effect of soluble factors released from microglia and macrophages have included human fetal brain directly infected with HIV, severe combined immunodeficiency mice cerebrocortical cultures inoculated with HIV infected human monocytes, and mixed rodent cerebrocortical cultures exposed to very low concentrations of the envelope protein HIV/gp120.

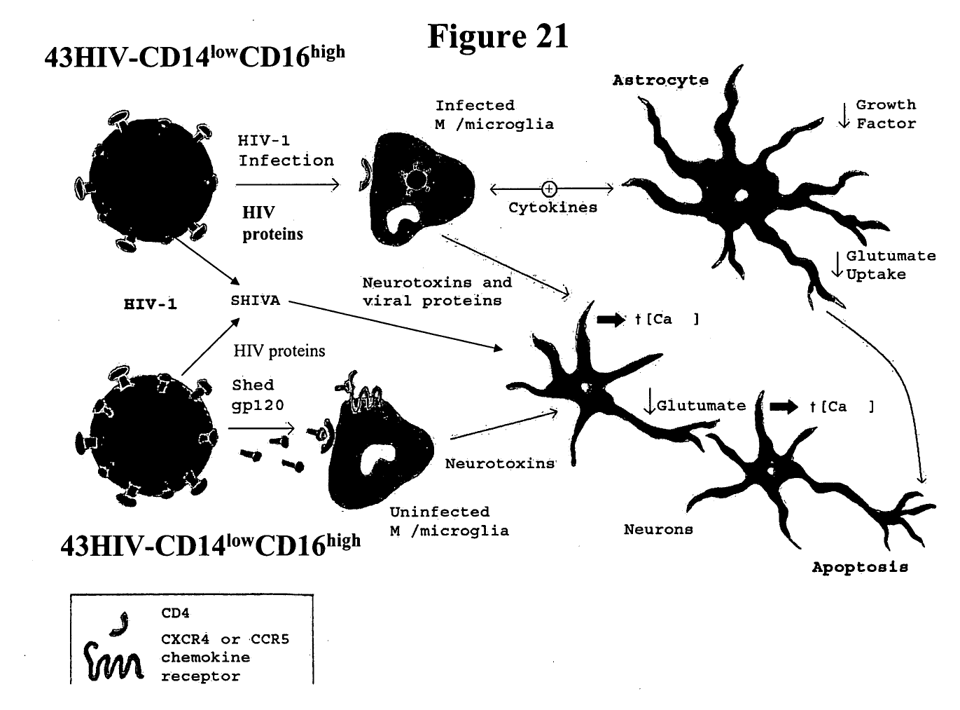

It has been suggested that the bone marrow is the site of the first steps leading to HIV dementia. The number of monocytes and resident macrophages increase in the bone marrow of AIDS patients. Infection of monocytes is also a feature of the later stages of disease, where it is associated with pathological changes in bone marrow and hematological abnormalities such as anemia. The events of late-stage infection, HIV replication in marrow, and/or the condition of chronic systemic inflammation also result in the activation of greater numbers of circulating monocytes. These activated monocytes are primed for transendothelial migration into the brain where their presence is associated with the onset of clinical disease. This activated monocytic population found in brain tissue expresses the CD14^{low}CD16^{high} phenotype. As correlation has been suggested between HAD and increased levels of circulating monocytes expressing CD14^{low}CD₁₆^{high}. This idea is further substantiated by the fact that acute SIV infection is associated with increases in both the number of circulating CD14^{low}CD16^{high} monocytes and the number of perivascular macrophages in the brain. Clone 43 is a human macrophage hybridoma cell line that expresses the CD14^{low}CD16^{high} phenotype and therefore represents a key cell in HAD. We have demonstrated that 43 cells produce SHIV A, a relatively neuroselective apoptotic protein 4 weeks after HIV infection, express gp 120 on their surface as well as produce TNF-α, and CXCR4 utilizing HIV isolates so that changes in the 43 cells after HIV infection mimic events that contribute to the neuronal dysfunction of HIV-1 infected patients. SHIV A may act directly as a neurotoxin to induce neuronal apoptosis or additively with TNF-α since it induces apoptosis through caspase-9 activation. The possible role of SHIV A in HAD is illustrated in FIG. 21.

### Example 9

### Treatment of Cancer

A composition of the present invention may also be used in the treatment of any cancer in which the SHIV A-based compositions may have an ameliorative effect through the induction of apoptosis. Initially, one may verify this ameliorative effect by contacting a model cancer cell line with the SHIV A-compositions of the present invention. Example 4 shows that SHIV A induced apoptosis in neuronal cell line SH SY5Y, thereby suggesting that cancer cell growth, proliferation and/or metastasis may be inhibited by SHIV A.

In light of the findings disclosed in Example 4, it is contemplated that the compositions of the present invention will likely prove effective in in vivo cancer treatment regimens. Initially, such in vivo regimens will preferably be corroborated in animal models of cancer, e.g., the nude mouse model. Once such studies have been used to verify the anti-cancer regimens, the compositions may be used in the treatment of an individual exhibiting a cancer. An “individual” as used herein, is a vertebrate, preferably a mammal, more preferably a human. Mammals include research, farm, and sport animals, and pets.

It is contemplated that the compositions of the present invention may be used in the treatment of numerous cancers, including but not limited to Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

The SH SY5Y cell line, which was used by the present inventors to verify the apoptotic activity of SHIV A in cancer cells, was initially developed from a bone marrow biopsy of a neuroblastoma patient whose primary thoracic tumor had metastasized (Biedler, et al. (1978) Cancer Res. 38, 3751-3757) and is well-recognized as a model cell line for neuroblastoma. Neuroblastoma is one of the most common pediatric solid tumors and frequently occurs during infancy, with the primary lesion in the adrenals and sympathetic chain and metastases to lymph nodes, liver, skin, and bone marrow. This tumor is difficult to treat as common modes of chemotherapy have harsh side effects on normal infant tissue. A variety of modalities have been used to treat neuroblastoma, such as surgery, radiotherapy, and chemotherapy, with varying degrees of success. For many patients, neuroblastoma continues to be fatal.

Cells within neuroblastoma tumors resemble those found in normally developing tissue of the sympathetic nervous system. Neuroblastomas may contain undifferentiated, closely packed spheroidal cells that closely resemble migrating neural crest cells of early embryos (neuroblasts), along with more differentiated cells whose immature nerve fibers tangle, thereby forming a rosette which is the first recognizable sign of neuronal differentiation. Some neuroblastomas undergo spontaneous regression or maturation to benign ganglioneuromas. The similarity of neuroblastoma cells to neuroblasts and the ability of neuroblastoma cells to spontaneously mature to a more benign form indicate that the disease may originate as the result of a block of differentiation of a sympathetic precursor cell.

Neuroblastoma may be treated with SHIV A protein compositions, with expression vectors that encode the SHIV A protein, or other agents that exert an effect on SHIV A protein expression and/or activity. These compositions are collectively referred to in these examples as “SHIV A-based therapeutic compositions.”

The SHIV A-based therapeutic compositions may be administered directly to the tumor site or may be delivered systemically to the individual. For systemic administration, the composition is typically administered orally or parenterally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, and infusion techniques. The SHIV A-based therapeutic compositions may be delivered to the patient alone, or indeed, in combination with other therapies used to combat the cancer. Where a combination therapy is contemplated, the SHIV A-based therapeutic compositions may be administered before, after or concurrently with the other anti-cancer agents.

In certain embodiments, tumor resection is performed. In addition to reducing the tumor mass, such resection facilitates intratumoral inoculation, in which patients are administered SHIV A-based therapeutic compositions via an Ommaya reservoir which will be placed at the time of tumor resection. Patients with either cystic tumors or those in which a subtotal or total resection can be accomplished are eligible for this form of therapeutic delivery.

In the gene therapy embodiments, during the immediate postoperative period and at appropriately spaced intervals afterward, patients are administered 10⁷-10¹⁰ plate forming units (PFU), and preferably 10⁸-10⁹ PFU of SHIV A encoding expression vector in 2-3 ml of sterile bacteriostatic PBS. A typical treatment course may comprise about six doses delivered over a 7 to 21 day period. Upon election by the clinician, the regimen may be continued as six doses every three weeks or on a less frequent (monthly, bimonthly, quarterly, etc.) basis. Of course, these are only exemplary times for treatment, and the skilled practitioner will readily recognize that many other time-courses-are possible. The infusions continue until radiographic tumor progression or treatment complications occur.

An alternative mode of delivery is via an intraarterial approach with blood brain barrier opening with hyperosmotic mannitol. Such a procedure is performed initially one week post-surgery. Patients whose tumors are supplied primarily by one of the major carotid or basilar artery are brought to the Angiography suite and a routine catheterization of the cerebral artery supplying the major tumor territory performed. After this is accomplished, hyperosmotic (1.6 M) mannitol is infused at a rate of 5 ml/min over 20-30 minutes. This results in a reversible opening of the blood brain barrier which lasts for several minutes after the infusion is completed. During this time, SHIV A-based therapeutic composition (e.g., 10⁸-10⁹ PFUs of SHIV A-encoding expression vector) diluted in 50 ml sterile normal saline is infused at a rate of 5-10 ml/min. At the completion of the infusion, the catheter is removed. The procedure is repeated every month until radiographic tumor progression or treatment complications occur.

Clinical responses may be defined by acceptable measure. For example, a complete response may be defined by the disappearance of all measurable disease for at least a month. A partial response may be defined by a 50% or greater reduction of the sum of the perpendicular diameters of all evaluable tumor nodules or at least 1 month with no tumor sites showing enlargement. Similarly, a mixed response may be defined by a reduction of perpendicular diameters of all measurable lesions by 50% or greater with progression in one or more sites.

Of course, the above-described treatment regimes may be altered in accordance with the knowledge gained from clinical trials. Those of skill in the art will be able to take the information disclosed in this specification and optimize treatment regimes based on the clinical trials described in the specification.

### Example 10

### Clinical Trials of the Use of SHIV A-based Therapeutic Compositions

This example is concerned with the development of human treatment protocols using the SHIV A-based therapeutic compositions of the present invention. Such drug treatment will be of use in the clinical treatment of various disorders in which it is desirable to induce apoptosis through the use of SHIV A-based therapeutic compositions. Such treatment will result in amelioration, inhibition, or other abrogation of the disease being treated. Such treatment will be particularly useful tools in anti-cancer therapy, for example, in treating patients with neuroblastoma, or any other cancers in which the SHIV A-based therapeutic compositions are expected to cause apoptosis. Such cancers may or may not be resistant to conventional chemotherapeutic regimens.

The various elements of conducting a clinical trial, including patient treatment and monitoring will be known to those of skill in the art in light of the present disclosure. By way of example, the following information is being presented as a general guideline for use in establishing SHIV A-based therapeutic compositions in clinical trials for neuroblastoma. However, it should be understood that these general guidelines may be adapted for the treatment of any disorder in which SHIV A-based therapeutic compositions may be used to produce a therapeutic outcome. Example of such other disorders, and SHIV A-based intervention thereof, are discussed throughout the specification and especially in Section 1.

Patients with diagnosed with neuroblastoma will be chosen for clinical study. In an exemplary clinical protocol, patients may undergo placement of a Tenckhoff catheter, or other suitable device, in the cavity produced upon tumor resection to allow administration of the therapeutic compositions. The SHIV A-based therapeutic compositions may be administered alone or in combination with another chemotherapeutic agent. The administration may be directly into the tumor, or in a systemic manner. The starting dose may be 0.5 mg/kg body weight. Three patients may be treated at each dose level in the absence of grade >3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the SHIV A-based therapeutic compositions and the additional anti-cancer drug exceed 5EU/kg for any given patient.

The SHIV A-based therapeutic composition and/or anti-cancer agent combination may be administered over a short infusion time or at a steady rate of infusion over a 7 to 21 day period. The infusion given at any dose level will be dependent upon the toxicity achieved after each. Hence, if Grade II toxicity is reached after any single infusion, or at a particular period of time for a steady rate infusion, further doses should be withheld or the steady rate infusion stopped until toxicity improves. Increasing doses of SHIV A-based therapeutic composition in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 analysis should be performed weekly. Cellularity, cytology, LDH, and appropriate markers in biological fluid from the patient (e.g., CEA, CA15-3, CA 125, p185) and in the cells (p185) may be assessed. Where measurable disease is present, tumor measurements are to be recorded every 4 weeks. Appropriate radiological studies should be repeated every 8 weeks to evaluate tumor response. An urinalysis may be performed every 4 weeks.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

What is claimed is:

1. A method of diagnosing HIV infection in a subject comprising:
a. obtaining a biological sample from said subject;
b. determining the increased expression of a SHIV A in said biological sample.

2. The method of claim 1, wherein said cell sample contains cells selected from the group consisting of macrophages, neuronal cells, central nervous system cells, microglial cells, glial cells, T-cells, and B-cells.

3. The method of claim 1, wherein said SHIV A protein comprises a sequence of SEQ ID NO:2.

4. The method of claim 1, wherein said SHIV A protein is a 6 kDa fragment of the protein of SEQ ID NO:2.

5. The method of claim 1, wherein said determining comprising assaying for the presence of a nucleic acid that encodes said SHIV A protein in said sample.

6. The method of claim 5, further comprising subjecting said sample to conditions suitable for amplifying said nucleic acid.

7. The method of claim 1, wherein said determining comprises contacting said sample with an antibody that binds immunologically to a SHIV A protein.

8. The method of claim 7, further comprising subjecting the sample to ELISA.

9. The method of claim 1, further comprising the step of comparing the expression of SHIV A with the expression of SHIV A in a non-HIV infected sample.

10. The method of claim 9, wherein said comparing comprises evaluating the level of expression of SHIV A.

11. The method of claim 9, wherein said comparing comprises evaluating the structure of the SHIV A gene, protein or transcript.

12. The method of claim 11, wherein said evaluating comprises performing an assay selected from the group consisting of sequencing nucleic acid hybridization, PCR, RNAase protection.

13. The method of claim 12, wherein said nucleic acid hybridization assay is performed using a microarray comprising oligonucleotides derived from the sequence of SEQ ID NO:1.

14. The method of claim 13, wherein said oligonucleotides are each at least 20 bases in length.

15. A composition comprising an isolated pblypeptide encoding a SHIV A protein having the sequence of SEQ ID NO:2 and an immunological adjuvant, or pharmaceutically acceptable carrier or diluent.

16. The composition of claim 15, further comprising a combination of one or more competitive inhibitor of apartyl protease, one or more nucleoside substrate reverse transcriptase inhibitor, or one or more non-nucleoside reverse transcriptase inhibitors

17. The composition of claim 16, wherein said competitive inhibitor of aspartyl protease is selected from the group consisting of saquinavir, indinavir, ritonavir, nelfinavir and amprenavir.

18. The composition of claim 16, wherein said nucleoside substrate reverse transcriptase inhibitor is selected from the group consisting of zidovudine, didanosine, stavudine, lamivudine, zalcitabine and abacavir.

19. The composition of claim 16, wherein said non-nucleoside reverse transcriptase inhibitor is selected from the group consisting of nevaripine, delavaridine and efavirenz.

20. The composition of claim 15, wherein said polypeptide is conjugated to a carrier molecule or a tag.

21. The composition of claim 20, wherein said carrier molecule is selected from the group consisting of KLH, and BSA.

22. A monoclonal antibody that binds immunologically to a SHIV A protein.

23. The monoclonal antibody of claim 22, wherein said monoclonal antibody binds to a protein of SEQ ID NO:2 or a fragment or variant thereof.

24. The monoclonal antibody of claim 23, wherein said monoclonal antibody binds to a 6 kDa fragment of the protein of SEQ ID NO:2.

25. The monoclonal antibody of claim 24, wherein said monoclonal antibody neutralizes the biological activity of the 6 kDa fragment of the protein of SEQ ID NO:2.

26. The monoclonal antibody of claim 22, wherein said monoclonal antibody does not bind immunologically to other human polypeptides.

27. The monoclonal antibody of claim 22, wherein said monoclonal antibody binds to non-human homologs of SHIV A.

28. The monoclonal antibody of claim 22, wherein said monoclonal antibody further comprises a detectable label.

29. The monoclonal antibody of claim 28, wherein said detectable label is selected from the group consisting of a fluorescent label, a chemiluminescent label, a radiolabel and an enzyme.

30. The monoclonal antibody of claim 22, wherein said monoclonal antibody is formulated into a pharmaceutical composition.

31. The monoclonal antibody of claim 22, wherein said monoclonal antibody is formulated into a diagnostic kit, said kit further comprising instructions for performing a diagnostic assay to determine the presence of an SHIV A protein.

32. A hybridoma cell that produces a monoclonal antibody that binds/immunologically to a SHIV A protein.

33. The hybridoma cell of claim 32, wherein said hybridoma produces a monoclonal antibody that does not bind to other human proteins.

34. The hybridoma cell of claim 32, wherein the antibody binds to a non-human homolog of a protein of SEQ ID NO:2.

35. A polyclonal antisera comprising antibodies which bind immunologically to a SHIV A protein.

36. A nucleic acid construct comprising a polynucleotide of SEQ ID NO:1 operably linked to a heterologous promoter.

37. The nucleic acid construct of claim 36, wherein said heterologous promoter is selected from the group consisting of CMV, RSV, SV40, UbC, EF1 alpha, and tetracycline inducible promoter.

38. The nucleic acid construct of claim 36, wherein said polynucleotide is positioned in an antisense orientation with respect to the heterologous promoter.

39. The nucleic acid construct of claim 36, further comprising the nucleic acids of a viral vector selected from the group consisting of retrovirus, adenovirus, adeno-associated virus, herpes virus, and vaccinia virus.

40. The nucleic acid construct of claim 36, wherein said nucleic acid construct is packaged in a liposome.

41. A method of altering apoptosis in a first cell, comprising altering the expression or processing of SHIV A protein in a second cell.

42. The method of claim 41, wherein said second cell is an HIV-infected cell, said first cell is a neuronal cell, and said altering comprises decreasing apoptosis in said first cell by inhibiting the expression or activity of SHIV A protein in said HIV-infected second cell.

43. The method of claim 41, wherein said second cell is an HIV-infected cell, said first cell is a B cell or a T cell, and said altering comprises decreasing apoptosis in said first cell by inhibiting the expression or activity of SHIV A protein in said HIV-infected second cell.

44. The method of claim 43, wherein said first cell is co-treated with HAART.

45. The method of claim 42, wherein apoptosis is decreased within said HIV-1 infected second cell.

46. The method of claim 42, wherein apoptosis is decreased in cells surrounding said HIV-1 infected second cell.

47. The method of claim 41, wherein said first cell is a hyperproliferative cell and said altering comprises increasing cell apoptosis in said first cell by increasing the expression, processing or activity of SHIV A protein in said second cell.

48. The method of claim 47, wherein apoptosis is increased within said second cell.

49. The method of claim 47, wherein apoptosis is increased in cells surrounding said second cell.

50. The method of claim 41, wherein said method is performed in an in vitro assay.

51. The method of claim 41, wherein said first cell and said second cell ate located within a mammalian organism and the method is performed in vivo.

52. The method of claim 42, wherein said inhibiting the expression of SHIV A in said second cell comprises contacting SHIV A produced by said second cell with an agent that binds to and/or inactivates said SHIV A.

53. The method of claim 42, wherein said inhibiting the expression of SHIV A in said second cell comprises contacting said second cell with a nucleic acid construct that reduces the expression of SHIV A in said second cell.

54. The method of claim 52, wherein said agent is a small molecule inhibitor, or an antibody preparation.

55. A method of ameliorating inflammatory disease in an individual comprising administering to said individual a composition comprising SHIV A, in an amount effective to deplete B-cells and/or T-cells in said individual.

56. The method of claim 55, wherein said B-cells and/or T cells are depleted as a result of apoptosis.

57. A transgenic non-human animal, wherein the neuronal cells of said animal comprises a gene, that encodes an SHIV A protein, under the control of a neuron-specific promoter.

58. The transgenic non-human animal of claim 57, wherein said animal exhibit dementia.

59. A recombinant host cell, wherein said cell is transformed with an expression construct comprising a nucleic acid that encodes SHIV A under the control of a promoter.

60. The recombinant host cell of claim 59, wherein said cell is a neuronal cell.

61. The recombinant host cell of claim 59, wherein said cell is a macrophage.

62. The recombinant host cell of claim 59, wherein said cell further expresses one or more HIV-related genes selected from the group consisting of tat, nef, rev, vpr, vpu, env, pol, gag, and vpf.

63. The recombinant host cell of claim 59, wherein said cell has been transformed to express said one or more HIV-related genes.

64. The recombinant host cell of claim 59, Wherein said expression construct further comprises nucleic acids sequences of said one or more HIV-related genes.

65. A method of treating a subject having HIV-associated dementia comprising administering a composition according to claim 15.

66. A method of determining the efficacy of an HIV treatment regimen comprising monitoring the expression of SHIV A in the subject receiving the HIV treatment prior to and after said treatment wherein a decrease in the expression of SHIV A after said treatment indicates that the treatment was effective in alleviating the symptoms of HIV infection.

67. A method for screening for agents that modulate apoptosis comprising:
a. providing a cell that expresses SHIV A;
b. contacting said cell with a candidate modulator; and
c. monitoring a change in the expression or activity of SHIV A that occurs in the presence of said modulator.

68. The method of claim 67, wherein said monitoring step comprises comparing the level of expression of said SHIV A in the presence of said modulator with the level of expression of said SHIV A in the absence of said modulator.

69. The method of claim 67, wherein said monitoring step comprises determining the level of secretion of a 6 kDa fragment of SHIV A in the presence of said modulator with the level of secretion of said 6 kDa fragment of SHIV A in the absence of said modulator.

70. The method of claim 67, wherein said monitoring step comprises comparing apoptosis of cells surrounding said cell in the presence of said modulator to the level of apoptosis of surrounding cells in the absence of said cell.

71. The method of claim 67, wherein said cell that expresses said SHIV A is a macrophage or microglial cell.

72. The method of claim 70, wherein said surrounding cells is a cell selected from the group consisting of a neuronal cell, a B-cell and a T-cell.

73. The method of claim 67, wherein said cell that expresses said SHIV A has been derived from a HIV-infected patient.

74. The method of claim 67, wherein said cell that expresses said SHIV A is a recombinant host cell according to claim 59.

75. The method of claim 67, wherein said contacting is performed in vitro.

76. The method of claim 67, wherein said cell that expresses said SHIV A is located within a mammalian organism and the screening method is performed in vivo.

77. The method of claim 67, wherein said cell that expresses said SHIV A is part of a transgenic, non-human animal.

78. The method of claim 67, wherein said candidate modulator is a nucleic acid construct that reduces the expression of SHIV A.

79. The method of claim 67, wherein said candidate modulator is an antibody.

80. The method of claim 79, wherein said antibody is a monoclonal antibody.

81. A composition comprising a candidate modulator of apoptosis identified according to a method of any one of claims 67.

82. A kit for determining the presence of a SHIV A protein in a sample, said kit comprising a monoclonal antibody of claim 22, and a composition comprising an SHIV A protein.

83. An apoptotic protein comprising the sequence of SEQ ID NO:3.

84. A nucleic acid that encodes the protein of claim 83.

85. An expression vector that comprises the nucleic acid of claim 84.

86. A host cell transformed with the expression vector of claim 85.
